(19) United States
(12) Patent Application Publication
(10) Pub. No.: US 2015/0320893 A1
(43) Pub. Date: Nov. 12, 2015

(54) TREATMENT OF LATENT HIV INFECTION

(71) Applicant: Joseph M VOLPE, Durham, NC (US)

(72) Inventor: Joseph Michael Volpe, Durham, NC (US)

(21) Appl. No.: 14/398,482

(22) PCT Filed: Mar. 14, 2013
(86) PCT No.: PCT/US2013/031616

§ 371 Date: Nov. 3, 2014

Related U.S. Application Data
(60) Provisional application No. 61/642,278, filed on May 3, 2012.
(60) Provisional application No. 61/729,054, filed on Nov. 21, 2012.

(51) Int. Cl. A61K 51/10; A61K 31/52; A61K 31/513; A61K 31/365; A61K 31/167; A61K 31/22; A61K 31/5365; A61K 31/47; A61K 31/19; A61K 38/15

(52) U.S. Cl. CPC A61K 51/1006; A61K 31/19; A61K 31/52; A61K 31/513; A61K 38/15; A61K 31/167; A61K 31/22; A61K 31/5365; A61K 31/47; A61K 31/365

(57) ABSTRACT

Methods for treating HIV positive patients, and purging and eradicating latent HIV virus from a patient's system, are disclosed. The bulk of viral load is eradicated using conventional antiretroviral (ARV) therapy. Compounds that encourage viral production in the latent cells are then administered, preferably without activating those cells, while maintaining the ARV therapy. The administration of compounds that encourage viral production in latent cells is cycled, and after around 7-10 cycles, the methods can virtually eliminate latent HIV in the patient. Ideally, the ARV regimen includes at least one integrase inhibitor, at least one entry inhibitor, such as a CCR5 antagonist, and at least one, and preferably two, reverse transcriptase inhibitors. The compounds that encourage viral production in latent cells ideally include a combination of prostratin or a prostratin analog and an HDAC inhibitor, such as butyrate, valproate, or SAHA.

### FIELD OF THE INVENTION

This invention is generally in the area of the treatment of HIV-1 infection, and, more particularly, relates to the treatment of latent HIV-1 infection. The methods generally involve continuous administration of HAART, while cycling the administration of compounds that activate HIV-1 gene expression in latent cells.

### BACKGROUND OF THE INVENTION

Combination antiretroviral therapy (cART) controls HIV-1 replication and delays disease progression through the actions of various antiretroviral (ARV) drugs that target different parts of the viral life cycle. However, virus reemerges rapidly after treatment interruption due to the existence of a latent viral reservoir. This reservoir is thought to consist mainly of latently infected resting memory CD4⁺ T cells. Due to the long half-life of this reservoir (44 months), it has been estimated that its total eradication with current treatment would require over 50 years.

Latently infected cells contain replication-competent integrated HIV-1 genomes that are blocked at the transcriptional level, resulting in the absence of viral protein expression. HIV-1 depends on both cellular and viral factors for efficient transcription of its genome, and the activity of the HIV-1 promoter is tightly linked to the level of activation of its host cell.

Reactivation of latently infected memory T cells by their cognate antigen leads to a reactivation of viral gene expression and the completion of the viral life cycle. However, it is not clear how the latent state is established. It has been proposed that latency occurs when an activated T cell in the early stage of infection returns to a quiescent state, leading to suppression of viral transcription until the cell becomes reactivated. However, the infected cell would need to survive the cytopathic effects of infection to effectively transition to the resting state. Alternatively, an activated, infected cell could become quiescent before the onset of viral expression and the occurrence of cytopathic effects, as has been reported during thymopoiesis. The unlikely coincidence of these two events could account for the low frequency of latently infected cells in vivo (about 10⁶ cells per infected individual).

The persistence of transcriptionally silent but replication-competent HIV-1 reservoirs in cART-treated infected patients represents a major hurdle to viral eradication. Activation of HIV-1 gene expression in these cells together with an efficient cART regimen has been proposed as an adjuvant therapy aimed at decreasing the pool of latent viral reservoirs. However, it would be desirable to have a method for doing so without activating those cells, and also for determining the appropriate duration of therapy.

The present invention provides such methods.

### SUMMARY OF THE INVENTION

In one embodiment, the methods described herein can be used to purge and eradicate the HIV-1 virus from a patient's system. While not wishing to be bound to a particular theory, it is believed that there are a finite and discreet number of cells harboring latent virus. By eradicating the bulk of viral load from the majority of cells using conventional cART and then administering compounds that encourage viral production in the latent cells, preferably without activating those cells, while maintaining the ARV therapy, one can eliminate HIV-1 in the patient.

The method involves three main components. The first is an ARV regimen that reduces viral loads to extremely low levels, so that the remaining virus is predominantly present in the latent cells. Then, while maintaining the ARV therapy, compounds are administered that encourage viral production in latent cells, preferably without activating those cells. The third element involves knowing when to administer each therapy, and this typically involves a series of cycles, where a first cycle involves the ARV therapy, and the next cycle involves both ARV therapy and administration of compounds that encourage viral production in the latent cells, and the cycles are repeated for a sufficient number of times that viral eradication is achieved. In one aspect of this method, a time-based schedule which dictates when these therapies are given is provided.

In one aspect of this embodiment, once viral production is encouraged in latent cells, the patient is treated with a cytotoxic antibody or similar agent which is targeted to HIV-1 infected cells, and delivers a toxic payload to the cells. In one aspect, the cytotoxic agent is a radioisotopically labeled antibody specific for HIV-1 infected cells, such as monoclonal antibodies to HIV's gp120 and gp41 envelope proteins tagged with bismuth 213 or rhenium 188 respectively, with bismuth 213 being particularly preferred. The antibody can be, for example, an antibody to the gp41 protein designed to bind to the 246-D region (a conserved sequence present across a wide range of genetically diverse HIV strains). This approach is known as radioimmunotherapy, or RIT. Radioimmunotherapy for HIV is disclosed, for example, in EP1868639A4, the contents of which are hereby incorporated by reference in their entirety for all purposes.

Other cytotoxic agents that can be used include maytansines, such as DM1 (mertansine) and DM4, auristatins, such as MMAE and MMAF, calicheamicin, duocarmycin, doxorubicin, and type 1 and 2 ribosome inactivating proteins (RIPs), such as trichosanthin, luffin, ricin, agglutinin, and abrin. In this aspect, the patient has been treated with cART to reduce viral loads, and the treatment with the cytotoxic antibody, such as RIT, does not affect the uninfected cells. The therapy, such as RIT, will also not likely treat quiescent cells with latent provirus, which is why compounds that stimulate viral production will be administered prior to or along with RIT or other such cytotoxic therapy. The co-administration of ARVs prevents any virus that is produced from further infecting any currently uninfected cells, and the RIT or other such cytotoxic therapy specifically targets and kills those cells that are producing virus. When administered according to the protocols described herein, the virus can be eradicated.

The cART regimen is typically selected from the available FDA approved ARVs that have a mechanism of action that interrupts the viral lifecycle prior to viral DNA integration into the host cell genome. In one embodiment, the regimen includes at least one integrase inhibitor, at least one entry inhibitor, such as a CCR5 antagonist, and at least one, and preferably two, reverse transcriptase inhibitors.

Ideally, the specific cART regimen includes multiple ARVs that are known to be effective in biological compartments outside of the lymph and serum, particularly the central nervous system, as not all drugs penetrate the CNS equally. In one aspect of this embodiment, the ARV regimen includes raltegravir, maraviroc, abacavir, and zidovudine (AZT), where elvitegravir or dolutegravir (assuming FDA approval) can be substituted for raltegravir.

The set of compounds that provoke viral replication in latently infected cells can include a mixture of compounds, some of which are already FDA approved and could be repurposed. These compounds can be used in conjunction with the ARVs as adjuvant therapy. That is, while the ARVs limit viral replication and integration, the adjuvants encourage latent cell depletion via viral production.

In one embodiment, the compounds that provoke viral replication in latently infected cells include at least prostratin, bryostatin, or one of the chemical analogues of prostratin (CAPs) or bryostatin (CABs) described herein, sodium butyrate (an HDAC inhibitor), and a drug typically approved for T-cell lymphomas, such as romidepsin or vorinostat (both are also HDAC inhibitors).

Using both the ARVs and the adjuvants, cycling the administration of the adjuvants, ideally according to a treatment schedule, will encourage latent viral production, thereby killing the cells harboring latent virus, and preventing new cellular infection. By killing off more cells per round of therapy than are being infected, the virus can eventually be purged from the body.

In one embodiment, the schedule for using both the ARVs and the adjuvents is as follows:

From time point zero, from when a patient is identified as a candidate for eradication therapy, the patient would be put on the daily ARV regimen alone for approximately 6 months. Candidates for ARV therapy would be those recently infected that are still naïve to ARVs, as well any patient for whom resistance testing indicates that the above described ARV regimen can be assembled such that all agents are fully active. All candidates ideally have an R5 tropic virus as indicated by a clinically validated tropism assay, so that the CCR5 antagonist component of the ARV regimen is effective.

In one embodiment, at approximately 6 months, provided that the patient has an undetectable viral load (<50 copies/mL), the patient would continue with the ARV regimen and would also begin taking the prostratin, bryostatin, or one or more CAP(s) or CAB(s) daily, along with a single HDAC inhibitor, such as sodium butyrate. This therapy would be continuous.

Additionally, the patient would receive, in cycles, a second HDAC inhibitor, such as romidepsin or vorinostat (SAHA). Where romidepsin or vorinostat are administered, an adjusted dose (rather than the one given for oncology) of romidepsin or vorinostat is used.

The treating physician would then periodically monitor the patient's viral load, for example, every 2 weeks, until the patient's viral load returns to undetectable levels (<50 copies/mL). At that point, the doctor would administer another dose of the second HDAC inhibitor (such as romidepsin or vorinostat).

In one aspect of this embodiment, the cycle of administration of romidepsin or vorinostat and viral load monitoring is continued for 7 rounds, 8 to 10 rounds, or up to 20 rounds. A schematic illustration of this embodiment is shown in FIG. 8.

In another embodiment, at approximately 6 months, provided that the patient has an undetectable viral load (<50 copies/mL), the patient would continue with the ARV regimen and would begin cycling in adjuvant therapy using prostratin, bryostatin, or one or more CAP(s) or CAB(s), along with one or more HDAC inhibitors, such as one or more of sodium butyrate, romidepsin and vorinostat. Where romidepsin or vorinostat are administered, an adjusted dose (rather than the one given for oncology) of romidepsin or vorinostat.

The treating physician would then periodically monitor the patient's viral load, for example, every 2 weeks, until the patient's viral load returns to undetectable levels (<50 copies/mL). At that point, the doctor would administer another dose of the adjuvant therapy.

In one aspect of this embodiment, the cycle of administration of adjuvant therapy and viral load monitoring is continued for 7 rounds, 8 to 10 rounds, or up to 20 rounds.

In either embodiment, at the end of seven rounds of therapy, patients can be checked by a single-copy VL assay to determine viral persistence. If virus is detected, three more cycles of therapy are recommended. If virus is not detected, then patients will be taken off all drugs, including the ARVs, and monitored with viral load tests every 2 weeks.

Patients with persistent absence of VL detection after 3 months may then be monitored less frequently for up to a year for re-emergence of virus. If no re-emergence is detected, then the virus has likely been purged from the patient's system.

If re-emergence is detected at any point, then the therapy may not work for this patient. Not all patients will be successful with this adjuvant approach. Ideally, the adjuvants specifically target latent CD4 cells in the lymph, lymph nodes, and secondary lymphoid tissues, including the spleen and GALT (gut-associated lymphoid tissue). Additionally, the 6-month lead-in therapy should ameliorate viral reduction in the CNS, the seminal compartment, and the macrophage and dendritic cell populations.

### BRIEF DESCRIPTION OF THE FIGURES

FIG. 1 Model of latent infection after start of HAART therapy. The dynamics of the total CD4 T cell population per mL are shown in the upper panels. Virus RNA per mL is shown in the middle panels. Central memory (solid line) transitional memory (dashed line), and total memory (dotted line) CD4 T cells per mL are shown in the lower panels. The left and right columns represent patients with high and low CD4 counts prior to the initiation of HAART. The first 500 days present model dynamics in the absence of HAART, and the time following 500 days represents the model dynamics in the presence of HAART. Parameter values used in simulations are presented in Table 1, a=0 and β=2×10−8 mL day-1 (left column); β=4.2×10−8 mL day-1 (right column)

FIG. 2 Model of viral blips due to antigen specific activation of latent cells. The dynamics of the total CD4 T cell population are shown in the left panel. Virus RNA is shown in the middle panel. Note that random activation events and times can bring the viral load above the limit of detection (dotted line). Central memory (solid line) and transitional memory (dashed line) CD4 T cells are shown in the right panel. Parameter values used in simulations are presented in Table 1, and a is chosen randomly from the interval (10-4, 10-2). The first 500 days present model dynamics in the absence of HAART and the time following 500 days represent the model dynamics in the presence of HAART

FIG. 3 Model of three rounds of immune activation therapy (IAT). Each treatment results in a spike in viral load (center panel), which is subsequently controlled, and a rapid decline in the size of the latent pools (right panel). The first 500 days represent model dynamics without HAART, the next 510 days represent HAART alone, and the last 990 days represent three rounds of IAT with the activation stages spanning 30 days and the relaxation stages spanning 300 days.

FIG. 4 Total CD4 T cell count (left panel), virus concentration (middle panel) and latent cell populations (right panel) under IAT+HAART (black) and under HAART alone (grey). We assume that IAT starts at 1010 days following infection and is consists of three rounds of IAT+HAART for 30 days followed by 300 days of HAART alone. At day 2000 HAART is interrupted. We notice a rebound in both virus and latent populations (black lines).

FIG. 5 Virus (left panel) and latent cells (right panel) dynamics under IAT+HAART and different levels of antiproliferative drugs

FIG. 6 The size of the basic reproductive number under nonspecific activation therapy (R′0) for different values of basic reproductive number in the absence of therapy (R0) and total resting cells activation rate (p′). For example, when R0=0.5, broad activation will lead to HAART failure when more than 15% of resting cells get activated.

FIG. 7 Virus (left panel) and latent cells (right panel) dynamics under two immune activation therapy regimes: three rounds of 30 days IAT+HAART and 300 days HAART alone (black) and one round of 90 days of IAT+HAART and 900 days of HAART alone. Note the additive feature of the regimes.

FIG. 8 is a chart showing the effect of cyclization of secondary therapy to promote viral propagation in latent cells, while maintaining ARV therapy, on the number of cells with latent virus and on the viral load (virions).

### DETAILED DESCRIPTION

Methods and compositions for purging and eradicating the HIV-1 virus from a patient's system are disclosed. The methods involve eradicating the bulk of viral load from the majority of cells using conventional antiretroviral (ARV) therapy and then administering compounds that encourage viral production in the latent cells, preferably without activating those cells, while maintaining the ARV therapy. Using this approach, one can reduce and/or eliminate HIV-1 in the patient's latent cells.

In addition to purging and eradicating the HIV-1 virus, the methods allow one to treat an HIV-1 infection. As used herein, the term “treat” a subject with an HIV infection means to kill cells within the subject that contain HIV, to reduce the number of HIV particles causing the infection in the subject, to prevent the HIV infection from spreading in the subject, to reduce the further spread of HIV infection in the subject, to prevent the establishment of HIV infection in the subject, to treat the HIV infection, to improve symptoms associated with HIV infection, to reduce or prevent opportunistic infection associated with HIV infection, and/or to eliminate the HIV infection. The treatments disclosed herein are also expected to reduce the likelihood of spread of HIV infection to new subjects.

The method involves three main components. The first is an ARV regimen that reduces viral loads to extremely low levels, so that the remaining virus is predominantly present in the latent cells. Then, while maintaining the ARV therapy, compounds are administered that encourage viral production in latent cells, preferably without activating those cells. The third element involves knowing when to administer each therapy, and this typically involves a series of cycles. The first cycle involves the ARV therapy, and the next cycle involves both ARV therapy and administration of compounds that encourage viral production in the latent cells (referred to herein as adjuvant therapy). The cycles are repeated for a sufficient number of times that significant, and, ideally, complete viral eradication is achieved. In one aspect of this method, a time-based schedule which dictates when these therapies are given is provided.

Each aspect of the treatment method is described in more detail below.

The set of compounds that provoke viral replication in latently infected cells can include a mixture of compounds, some of which are already FDA approved and can be repurposed. These compounds can be used in conjunction with the ARVs as adjuvant therapy. That is, while the ARVs limit viral replication and integration, the adjuvants encourage latent cell depletion via viral production.

In one embodiment, the compounds that provoke viral replication in latently infected cells include at least one of the chemical analogues of prostratin (CAPs) or chemical analogues of bryostatin (CABs) (Stanford University), sodium butyrate (an HDAC inhibitor), and a drug typically approved for T-cell lymphomas, such as romidepsin or vorinostat (both are also HDAC inhibitors).

Using both the ARVs and the adjuvants, cycling the administration of the adjuvants, ideally according to a treatment schedule, will encourage latent viral production, thereby killing the cells harboring latent virus, and preventing new cellular infection. By killing off more cells per round of therapy than are being infected, the virus can eventually be purged from the body.

In one embodiment, the schedule for using both the ARVs and the adjuvants is as follows:

From time point zero, from when a patient is identified as a candidate for eradication therapy, the patient would be put on the daily ARV regimen alone for 6 months. Candidates for ARV therapy would be those recently infected that are still naïve to ARVs, as well any patient for whom resistance testing indicates that the above described ARV regimen can be assembled such that all agents are fully active. All candidates must also have an R5 tropic virus as indicated by a clinically validated tropism assay so that the CCR5 antagonist component of the ARV regimen is fully effective.

At 6 months, provided that the patient has an undetectable viral load (<50 copies/mL), the patient would continue with the ARV regimen and would also begin taking sodium butyrate and CAP(s) daily.

Additionally, a further HDAC inhibitor is cycled in with the ARV/CAP/sodium butyrate therapy. The further HDAC inhibitor can be, for example, romidepsin or vorinostat. Where romidepsin or vorinostat are administered, the patient can receive an adjusted dose (rather than the one given for oncology) of romidepsin or vorinostat appropriate for antiviral therapy. Those of skill in the art appreciate appropriate doses for such agents.

The provider would then monitor the patient's viral load every 2 weeks until the patient's viral load returns to undetectable (<50 copies/mL). At that point, the doctor would administer another dose of the romidepsin or vorinostat (or other HDAC inhibitor).

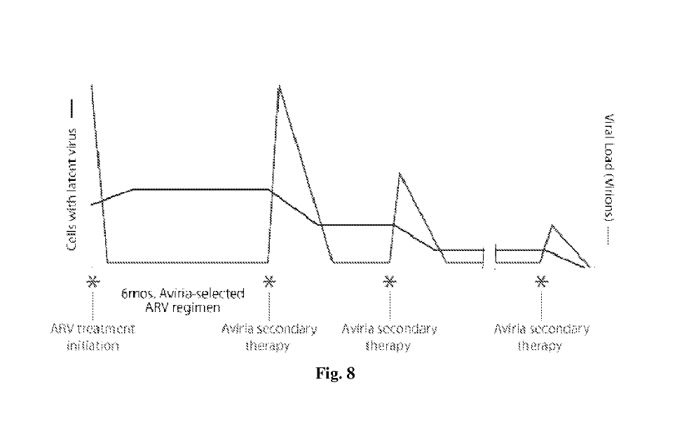

In one embodiment, the cycle of administration of romidepsin or vorinostat (or other HDAC inhibitor) and viral load monitoring is continued for 7 rounds, 8 to 10 rounds, or up to 20 rounds. A schematic illustration of this embodiment is shown in FIG. 8.

In another embodiment, at approximately 6 months, provided that the patient has an undetectable viral load (<50 copies/mL), the patient would continue with the ARV regimen and would begin cycling in adjuvant therapy using prostratin, bryostatin, or one or more CAP(s) or CAB(s), along with one or more HDAC inhibitors, such as one or more of sodium butyrate, romidepsin and vorinostat. Where romidepsin or vorinostat are administered, an adjusted dose (rather than the one given for oncology) of romidepsin or vorinostat is administered.

The treating physician would then periodically monitor the patient's viral load, for example, every 2 weeks, until the patient's viral load returns to undetectable levels (<50 copies/mL). At that point, the doctor would administer another dose of the adjuvant therapy.

In both embodiments, the cycle of administration of adjuvant therapy and viral load monitoring is continued for 7 rounds, 8 to 10 rounds, or up to 20 rounds. In either embodiment, the cycling of adjuvant therapy can be a single dosage, or multiple doses over a period of one day, two days, three days, four days, five days, six days, seven days, up to two weeks, up to three weeks, or up to one month. In one aspect of this embodiment, each cycle of adjuvant therapy is administered for a time between one week and ten weeks, with single or multiple doses of each agent per day.

At the end of seven rounds of therapy, patients can be checked by a single-copy VL assay to determine viral persistence. If virus is detected, three more cycles of therapy are recommended. If virus is not detected, then patients will be taken off all drugs, including the ARVs, and monitored with viral load tests every 2 weeks.

Patients with persistent absence of VL detection after 3 months may then be monitored less frequently for up to a year for re-emergence of virus. If no re-emergence is detected, then the virus has likely been purged from the patient's system.

If re-emergence is detected at any point, then the therapy may not work for this patient. Not all patients will be successful with this adjuvant approach.

The adjuvants specifically target latent CD4 cells in the lymph, lymph nodes, and secondary lymphoid tissues, including the spleen and GALT (gut-associated lymphoid tissue). Additionally, the 6-month lead-in therapy should ameliorate viral reduction in the CNS, the seminal compartment, and the macrophage and dendritic cell populations.

### DEFINITIONS

As used herein, the definition of the terms used herein are usually found in the art encyclopedias and dictionaries, see for example, Encyclopedia of Chemical Technology (all volumes), Hawley's Condensed Chemical Dictionary, etc.

“Alkyl (C₁-C₁₅)” refers to an alkyl group having from 1 (methyl) to 15 carbons in linear or branched chain. “Cyclic alkyl (C₃ to C₁₅)” refers to a cyclic group of from 3 to 15 carbon atoms. “Aromatic ring” refers to a carbocyclic or heterocyclic ring possessing resonance, namely it pertains to a closed ring of from 3 to 10 covalently linked atoms, more preferably from 5 to 8 covalently linked atoms, which ring is aromatic.

“Cytotoxic antibody” refers to an antibody that is chemically linked to a cytotoxin that causes a cell to apoptose or otherwise cease to function and denature. Such cytoxins include, but are not limited to, maytansines, such as DM1 (mertansine) and DM4, auristatins, such as MMAE and MMAF, calicheamicin, duocarmycin, doxorubicin, type 1 and 2 ribosome inactivating proteins (RIPs), such as trichosanthin, luffin, ricin, agglutinin, abrin, and radioactive elements such as bismuth 213 or rhenium 188.

“Daphnane” refers to a compound having a partial structure, which includes a tricyclic carbon skeleton shown below (with numbering).

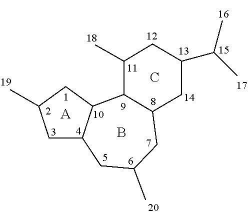

“Derivative” refers to a compound derived from another compound through one or more chemical transformations.

“Tigliane” refers to a compound having a partial structure, which includes a tetracyclic carbon skeleton shown below (with numbering).

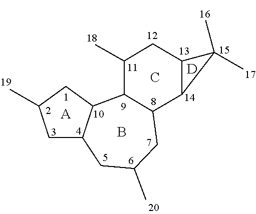

“Ingenane” refers to a compound having a partial structure which includes a tetracyclic carbon skeleton shown below (with numbering)

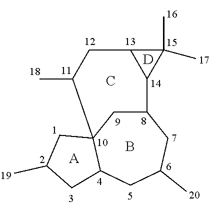

“Functional analog” refers to a compound that exhibits the same or similar activity (biological function) as another compound whether or not the compounds are structurally similar. For example, all protein kinase C(PKC) activators are functional analogs; even though they possess different structures they all activate PKC.

“Structural analog” refers to a compound that is structurally similar to another compound whether or not the compounds are functionally similar. For example, all tiglianes are structural analogs; even though they possess the same tigliane core they often exhibit widely different activities (functions).

“Tigliane-type compound” refers to a compound having at least a partial structure that includes the C- and D-rings of a tigliane, where R₁-R₁₄ can be varied. For example, Ingenanes are “Tigliane-type” compounds:

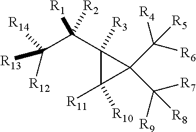

wherein R₁ to R₁₄ are the same or different and each independently selected from hydrogen, methyl, alkyl (C₁ to C₂₀), cyclic alkyl (C₃ to C₁₅) aromatic ring, hydroxyl, alkyl carbonate, carbamate, ester, ether, thiol, amine, or amide. R₁ may be alkanoyl as in —C(O)Ak wherein Ak is an alkyl chain (C₁ to C₂₀). R₁₋₁₄ groups may contain one or more heteroatoms including, but not limited to boron, nitrogen, oxygen, phosphorous, sulfur, silicon or selenium. R₁₁ and R₁₂ may be connected as in the case of tiglianes, or may be disconnected as in the case of 12-deoxy tigliane compounds which are structural or functional analogs of the illustrated embodiments.

Latent Cell Types

The methods described herein are designed to target latent cells where HIV-1 resides in provirus form. Such cells are predominantly CD4+ cells. These cells are primarily located in the lymph, lymph nodes and secondary lymphoid tissues, including the spleen and GALT (gut-associated lymphoid tissue). Additionally, latent cells reside in the CNS, the seminal compartment, and the macrophage and dendritic cell populations.

ARV/HAART

The ARV regimen is typically selected from the available FDA-approved ARVs that have a mechanism of action that interrupts the viral lifecycle prior to viral DNA integration into the host cell genome.

In one embodiment, the regimen includes at least one integrase inhibitor, at least one entry inhibitor, such as a CCR5 antagonist, and at least one, and preferably two, reverse transcriptase inhibitors.

Ideally, the specific antiretroviral regimen includes multiple ARVs that are known to be effective in biological compartments outside of the lymph and serum, particularly the central nervous system, as not all drugs penetrate the CNS equally. In one aspect of this embodiment, the ARV regimen includes raltegravir, maraviroc, abacavir, and zidovudine (AZT), where elvitegravir or dolutegravir can be substituted for raltegravir.

Protease Inhibitors

Representative protease inhibitors include Invirase® (Hoffmann-La Roche), Fortovase® (Hoffmann-La Roche), Norvir® (Abbott Laboratories), Crixivan® (Merck & Co.), Viracept® (Pfizer), Agenerase® (GlaxoSmithKline), Kaletra® (Abbott), Lexiva® (GlaxoSmithKline), Aptivus® (Boehringer Ingelheim), Reyataz® (Bristol-Myers Squibb), brecanavir (GlaxoSmithKline), and Prezista™ (Tibotec).

Nucleoside Reverse Transcriptase Inhibitors (NRTIs)

Representative nucleoside/nucleotide reverse transcriptase inhibitors (NRTIs) include Retrovir® (GlaxoSmithKline), Epivir® (GlaxoSmithKline), Combivir® (GlaxoSmithKline), Trizivir® (GlaxoSmithKline), Ziagen® (GlaxoSmithKline), Epzicom™ (GlaxoSmithKline), Hivid® (Hoffmann-La Roche), Videx® (Bristol-Myers Squibb), Entecavir (Bristol-Myers Squibb), Videx® EC (Bristol-Myers Squibb), Zerit® (Bristol-Myers Squibb), Viread™ (Gilead), Emtriva® (Gilead), Truvada® (Gilead), Atripla™ (Gilead/BMS/Merck), Alovudine (Boehringer), Elvucitabine (Achillion), KP-1461 (Koronis), Racivir (Pharmasset), Decelvuecitabine (Reverset) (Pharmasset), tenofovir disoproxil fumarate (DF) (Gilead) and GS9148 and prodrugs thereof (Gilead Sciences).

Non-Nucleoside Reverse Transcriptase Inhibitors (NNRTIs)

Representative non-nucleoside reverse transcriptase inhibitors (NNRTIs) include Viramune® (Boehringer Ingelheim), Rescriptor® (Pfizer), Sustiva® (Bristol-Myers Squibb), (+)-calanolide A (Sarawak Medichem), capravirine (Pfizer), DPC-083 (Bristol-Myers Squibb), TMC-125 (Tibotec-Virco Group), TMC-278 (Tibotec-Virco Group), IDX12899 (Idenix), and IDX12989 (Idenix).

Integrase Inhibitors

Integrase inhibitors block the action of integrase, a viral enzyme that inserts the viral genome into the DNA of the host cell. Since integration is a vital step in retroviral replication, blocking it can halt further spread of the virus. Representative integrase inhibitors include Raltegravir, Elvitegravir, Dolutegravir, GS 9137 (Gilead), MK-2048, globoidnan A, L-000870812, S/GSK1349572, S/GSK1265744, with or without a pharmacokinetic (PK) booster such as ritonavir or Gilead's pharmacoenhancing agent (also referred to as a PK booster), GS 9350.

Additional integrase inhibitors include those described in:

U.S. patent application Ser. No. 11/595,429, U.S. patent application Ser. No. 11/561,039, U.S. patent application Ser. No. 11/599,580, U.S. patent application Ser. No. 11/754,462, U.S. patent application Ser. No. 11/768,458, U.S. patent application Ser. No. 12/132,145, U.S. patent application Ser. No. 11/505,149, U.S. patent application Ser. No. 11/590,637, U.S. patent application Ser. No. 12/162,975, U.S. patent application Ser. No. 11/767,021, U.S. patent application Ser. No. 12/042,628, U.S. patent application Ser. No. 12/169,367, U.S. patent application Ser. No. 10/587,857, U.S. patent application Ser. No. 11/500,387, U.S. patent application Ser. No. 12/097,859, U.S. patent application Ser. No. 11/807,303, U.S. patent application Ser. No. 10/587,601, U.S. patent application Ser. No. 10/592,222, U.S. patent application Ser. No. 11/992,531, U.S. patent application Ser. No. 10/587,682, U.S. patent application Ser. No. 11/641,508, U.S. patent application Ser. No. 11/435,671, U.S. patent application Ser. No. 11/804,041, U.S. patent application Ser. No. 11/880,854, U.S. patent application Ser. No. 10/585,504, U.S. patent application Ser. No. 11/579,772, U.S. patent application Ser. No. 10/591,914, U.S. patent application Ser. No. 11/629,153, U.S. patent application Ser. No. 12/043,636, PCT WO 2007/019098, U.S. patent application Ser. No. 12/306,198, U.S. patent application Ser. No. 12/274,107, U.S. patent application Ser. No. 12/215,605, U.S. patent application Ser. No. 12/097,859, U.S. patent application Ser. No. 11/658,419, U.S. patent application Ser. No. 12/215,601, U.S. patent application Ser. No. 12/217,496, U.S. patent application Ser. No. 12/340,419, U.S. patent application Ser. No. 12/195,161, U.S. patent application Ser. No. 12/208,952, U.S. patent application Ser. No. 12/147,220, U.S. patent application Ser. No. 12/147,041, U.S. patent application Ser. No. 12/215,266, U.S. patent application Ser. No. 12/204,174, U.S. patent application Ser. No. 10/585,504, U.S. patent application Ser. No. 11/853,606, U.S. patent application Ser. No. 11/644,811, U.S. patent application Ser. No. 10/586,627, U.S. patent application Ser. No. 11/435,671, U.S. patent application Ser. No. 11/190,225, U.S. patent application Ser. No. 10/511,182, U.S. patent application Ser. No. 11/033,422, U.S. patent application Ser. No. 11/040,929, U.S. patent application Ser. No. 10/423,496, U.S. patent application Ser. No. 10/424,130, U.S. patent application Ser. No. 10/944,118, U.S. patent application Ser. No. 10/903,288, U.S. patent application Ser. No. 10/757,141, U.S. patent application Ser. No. 10/757,122, U.S. patent application Ser. No. 10/687,373, U.S. patent application Ser. No. 10/687,374, U.S. patent application Ser. No. 10/424,186, U.S. patent application Ser. No. 11/820,444, U.S. patent application Ser. No. 11/047,229, and U.S. patent application Ser. No. 11/827,959.

Additional integrase inhibitors include L-870,810 (Merck), INH-001 (Inhibitex), L870810 (Merck), PL-2500, composed of pryidoxal 1-5-phosphate derivatives (Procyon) monophores (Sunesis), V-165 (Rega Institute, Belgium), Mycelium integrasone (a fungal polyketide, Merck), GS 9224 (Gilead Sciences), AVX-I (Avexa), ITI-367, an oxadiazol pre-integrase inhibitor (George Washington University), GSK364735 (GSK/Shionogi), GS-9160 (GSK), S-1360 (Shionogi-GlaxoSmithKline Pharmaceuticals LLC), RSC 1838 (GSK/Shionogi), GS-9137 (taken alone or with Norvir) (Gilead), MK-2048 (Merck), S/GSK 1349572 and S/GSK 1265744 (no need for a PK booster) (GSK/Shionogi), 6-(3-chloro-2-fluorobenzyl)-1-[(2S)-1-hydroxy-3-methylbutan-2-y-l]-7-methoxy-4-oxo-1,4-dihydroquinoline-3-carboxylic acid (U.S. Patent Application Publication No. 20090018162), S-1360, L-870810, MK-0518 (Merck), C-2507 (Merck), BMS 538158 (Bristol Myers Squibb), and L-900564 (Merck).

The structure of L-900564 is shown below:

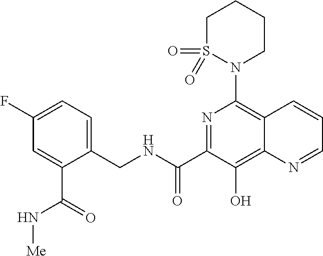 [SMILES: CNC(=O)c1cc(F)ccc1CNC(=O)c1nc(N2CCCCS2(=O)=O)c2cccnc2c1O]

Nair et al., J Med Chem. 2006 Jan. 26; 49(2): 445-447, discloses the following integrase inhibitors:

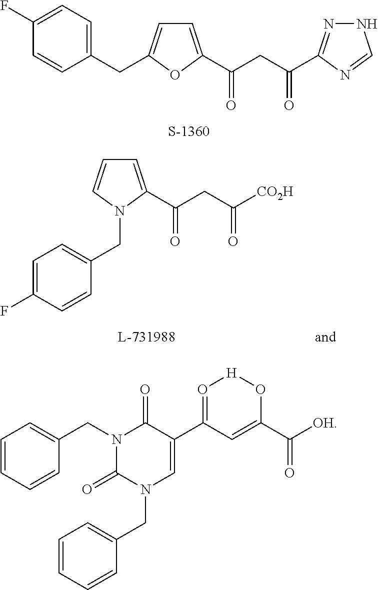

Additional integrase inhibitors are disclosed in Pais et al., J Med Chem. 2002 Jul. 18; 45(15):3184-94.

Several integrase inhibitors are peptides, including those disclosed in Divita et al., Antiviral Research, Volume 71, Issues 2-3, September 2006, Pages 260-267.

Another integrase inhibitor that can be used in the methods of treatment described herein include 118-D-24, which is disclosed, for example, in Vatakis, Journal of Virology, April 2009, p. 3374-3378, Vol. 83, No. 7.

Additional integrase inhibitors include those described in McKeel et al., “Dynamic Modulation of HIV-1 Integrase Structure and Function by Cellular LEDGF Protein, JBC Papers in Press. Published on Sep. 18, 2008 as Manuscript M805843200.

Other representative integrase inhibitors include dicaffeoylquinic acids (DCQAs), such as those disclosed in Zhu et al., “Irreversible Inhibition of Human Immunodeficiency Virus Type 1 Integrase by Dicaffeoylquinic Acids,” Journal of Virology, April 1999, p. 3309-3316, Vol. 73, No. 4.

There are also various nucleoside compounds active as integrase inhibitors, including those disclosed in Mazumder, A., N. Neamati, J. P. Sommadossi, G. Gosselin, R. F. Schinazi, J. L. Imbach, and Y. Pommier. 1996. Effects of nucleotide analogues on human immunodeficiency virus type 1 integrase. Mol. Pharmacol. 49:621-628.

Cellular Inhibitors

A representative cellular inhibitor is Droxia® (Bristol-Myers Squibb).

Entry Inhibitors

Entry inhibitors, also known as fusion inhibitors, are a class of antiretroviral drugs, used in combination therapy for the treatment of HIV-1 infection. This class of drugs interferes with the binding, fusion and entry of an HIV-1 virion to a human cell. By blocking this step in HIV-1's replication cycle, such agents slow the progression from HIV-1 infection to AIDS.

Representative entry inhibitors (including fusion inhibitors) include Fuzeon™ (enfuvirtide, Trimeris), T-1249 (Trimeris), AMD-3100 (AnorMED, Inc.), CD4-IgG2 (Progenics Pharmaceuticals, BMS-488043 (Bristol-Myers Squibb), aplaviroc (GlaxoSmithKline), Peptide T (Advanced Immuni T, Inc.), TNX-355 (Tanox, Inc.), and maraviroc (Pfizer).

CXCR4 Inhibitors

Representative CXCR4 Inhibitors include AMD070 (AnorMED, Inc.), AMD-3100 (AnorMED, Inc.), and Ibalizumab.

CCR5 Antagonists

CCR5 receptor antagonists are a class of small molecules that antagonize the CCR5 receptor. The C-C motif chemokine receptor CCR5 is involved in the process by which HIV-1, the virus that causes AIDS, enters cells. Hence antagonists of this receptor are entry inhibitors and have potential therapeutic applications in the treatment of HIV-1 infections. A representative CCR5 antagonist is vicriroc

(Schering-Plough). Other compounds include maraviroc, aplaviroc and vicriviroc (Schering-Plough), INCB009471 (Incyte), and cenicriviroc (Tobira Therapeutics).

Other agents are under investigation for their ability to interact with the proteins involved in HIV-1 entry and the possibility that they may serve as entry inhibitors. These include TNX-355, a monoclonal antibody that binds CD4 and inhibits the binding of gp120, PRO 140, a monoclonal antibody that binds CCR5, BMS-488043, a small molecule that interferes with the interaction of CD4 and gp120, Epigallocatechin gallate, a substance found in green tea, b12, an antibody against HIV-1 found in some long-term non-progressors, Griffithsin, a substance derived from algae, DCM205, a small molecule based on L-chicoric acid, an integrase inhibitor, CD4 specific Designed Ankyrin Repeat Proteins (DARPins), which potently block viral entry of diverse strains and are being developed and studied as potential microbicide candidates.

The HAART or ARV used in the methods described herein ideally includes In one embodiment, the regimen includes at least one integrase inhibitor, at least one entry inhibitor, such as a CCR5 antagonist, and at least one, and preferably two, reverse transcriptase inhibitors.

Ideally, the specific antiretroviral regimen includes multiple ARVs that are known to be effective in biological compartments outside of the lymph and serum, particularly the central nervous system, as not all drugs penetrate the CNS equally. In one aspect of this embodiment, the ARV regimen includes raltegravir, maraviroc, abacavir, and zidovudine (AZT), where elvitegravir or dolutegravir can be substituted for raltegravir.

### Prostratin and Analogs Thereof

In addition to the HAART/ARV, the methods involve cycling in treatment with prostratin or an analog thereof, and a histone deacetylase inhibitor (HDAC inhibitor). Prostratin analogs are described, for example, in U.S. Publication No. 2012/0101283 by Wender et al., the contents of which are hereby incorporated by reference for all purposes.

In one embodiment, the prostratin analogs are 12-deoxy tigliane-type compounds or structural or functional analogs thereof, of the formula:

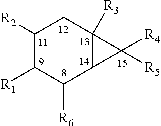

wherein R₁, R₂, R₃, R₄, R₅, and R₆ are each independently selected from the group consisting of hydrogen, alkyl (C₁ to C₁₅), cyclic alkyl (C₃ to C₁₅), aromatic ring, hydroxyl, carbonate, carbamate, ester, ether, thiol, amine, amide, guanidine or urea, wherein the R₁₋₆ groups may be straight-chained or branched, wherein the R₁₋₆ groups may comprise one or more heteroatoms including, but not limited to boron, nitrogen, oxygen, phosphorous, sulfur, silicon or selenium, and wherein R₁ and R₆ may be connected as in the case of 12-deoxy tigliane-type compounds, or may be disconnected as in the case of structural or functional analogs.

Preferably, R₂ is methyl, R₃ is an ester (—OC(O)Ak, where Ak is an alkyl chain), R₄ and R₅ are methyl groups, and R₁ and R₆ are connected by a 5 member alkyl chain as in the tigliane skeleton.

In one aspect of this embodiment, the 12-deoxy tigliane-type compound is prostratin or 12-deoxyphorbol-13-phenylacetate (DPP).

In some embodiments, R₁, R₂=H, R₃=OAc (Prostratin); R₁, R₂=H, R₃=OCOCH₂PH (DPP); R₂ and/or R₃=H (di-deoxy tiglianes); OCOR (esters); OR (ethers); Cl, Br, I, F (halogens); SeR (selenium ethers); SAk, SOAk, SO₂Ak (thiol ethers, sulfones, sulfonates); Ak, Ar, CN (Carbon substituents); and NHR, NR₂, NHCOR (Amines, amides). Additional analogs can be prepared by modifying the a, b, c, and/or d rings. The pyrazoline ring can be functionalized with various functional groups, and also converted to cyclopropane derivatives.

Representative compounds are shown below:

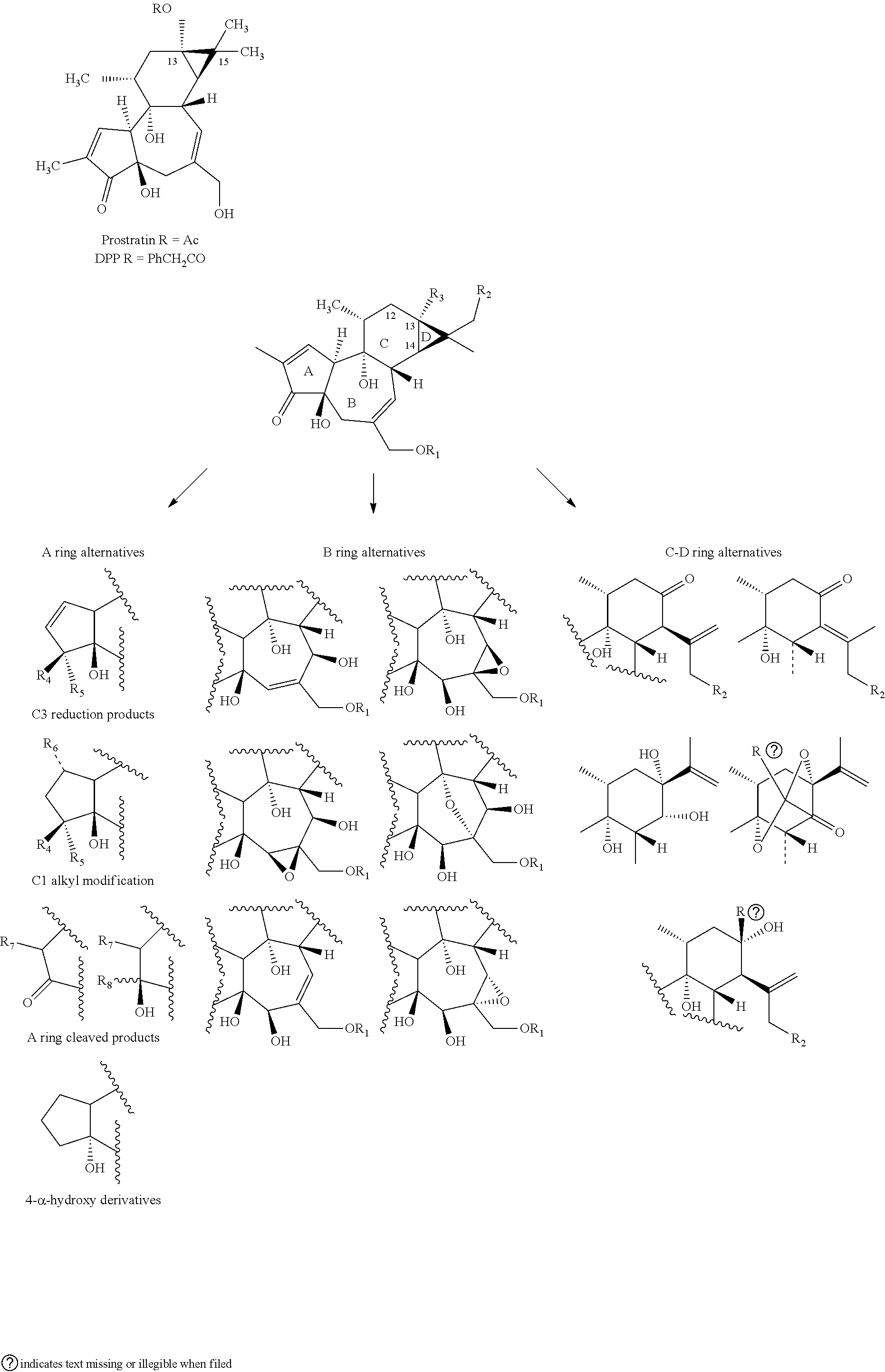

In addition to allowing for introduction of various groups at the C13 position, the methodology shown above can also be used to prepare a wide range of varied structures of the tigliane, daphnane and ingenanes families and structural and functional analogs thereof.

Another analog is 12-deoxyphorbol-13-phenylacetate (DPP).

Methods for obtaining prostratin and its analogs include the current isolation method from natural stemwood of Croton oil (from the seed of source Homalanthus nutans Croton tiglium) (Samoan mamala tree), which is available only in Samoa. The process involves extraction from 5-7 steps from Croton oil natural source, and a series of chromatographic purifications, with an overall yield of around 0.0013% (15 mg from ˜0.1% from Croton oil, 1.05 kg of stemwood, (˜10-20% from phorbol) varies significantly between samples).

Bryostatin and Analogs Thereof

Bryostatins are a group of macrolide lactones isolated from extracts of a species of bryozoan, Bugula neritina. There are numerous known bryostatins, and the structure of bryostatin 1 is shown below:

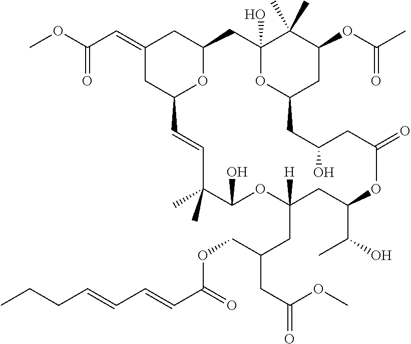

Representative bryostatin analogs (CABs) include those described in U.S. Pat. No. 6,624,189. In one embodiment of the analogs described in the '189 patent, the bryostatin analogues are represented by Formula I:

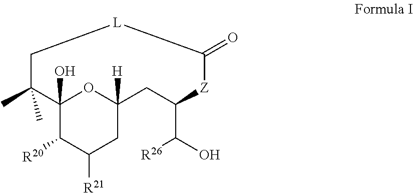

wherein: R²⁰ is H, OH, or O₂CR′; R²¹ is ═CR^{a}R^{b} or R²¹ represents independent moieties R^{c} and R^{d} where: R^{a} and R^{b} are independently H, CO₂R′, CONR^{c}R^{d} or R′; R^{c} and R^{d} are independently H, alkyl, alkenyl or alkynyl, or (CH₂)ₙCO₂R′ where n is 1, 2 or 3; R²⁶ is H, OH or R′; each R′ being independently selected from the group: alkyl, alkenyl or alkynyl, or aryl, heteroaryl, aralkyl or heteroaralkyl; L is a straight or branched linear, cyclic or polycyclic moiety, containing a continuous chain of preferably from 6 to 14 chain atoms, which substantially maintains the relative distance between the C1 and C17 atoms and the directionality of the C1C2 and C16C17 bonds of naturally-occurring bryostatin; and Z is —O— or —N(H)—; and the pharmaceutically acceptable salt thereof.

In one aspect of this embodiment, the recognition domain in this embodiment R²⁶ is H or methyl, particularly when R²¹ is ═C(H)CO₂R′. Especially preferred are the compounds where R²⁶ is H. A preferred upper limit on carbon atoms in any of R^{d}, R^{e} and R′ is about 20, more preferably about 10 (except as otherwise specifically noted, for example, with reference to the embodiment of the invention where a preferred R²⁰ substituent has about 9 to 20 carbon atoms). In a preferred aspect of the spacer domain of this embodiment, L contains a terminal carbon atom that, together with the carbon atom corresponding to C17 in the native bryostatin structure, forms a trans olefin.

In another embodiment, the bryostatin analogues are represented by Formulae II-V:

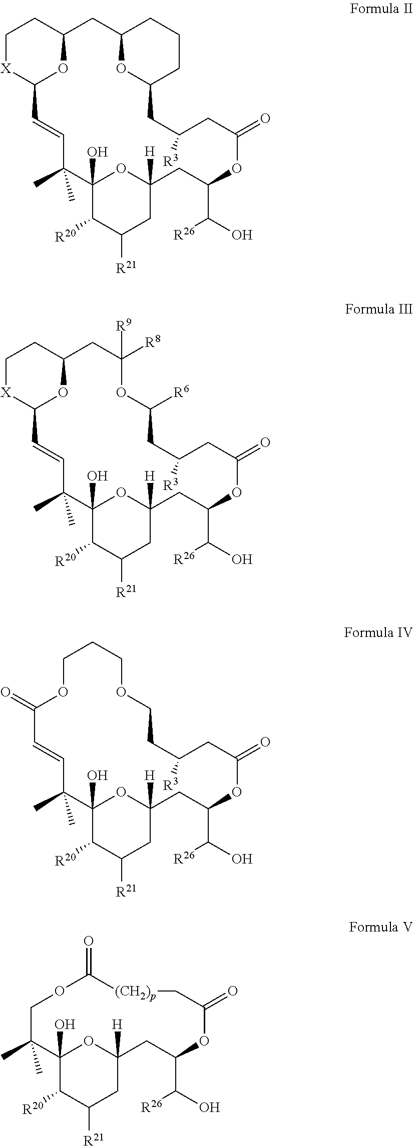

wherein: R³ is H, OH or a protecting group; R⁶ is H, H or ═O; R⁸ is selected from the group: H, OH, R′, —(CH₂)ₙO(O)CR′ or (CH₂)ₙCO₂-haloalkyl where n is 0, 1, 2, 3, 4 or 5; R⁹ is H or OH; R²⁰, R²¹, R²⁶ and R′ are as defined above with respect to Formula I; p is 1, 2, 3 or 4; and X is C, O, S or N—R^{e} where R^{e} is COH, CO₂R′ or SO₂R′, and the pharmaceutically acceptable salts thereof.

In one aspect of this embodiment, the CAB is a C26 des-methyl analogue of Formula IIa:

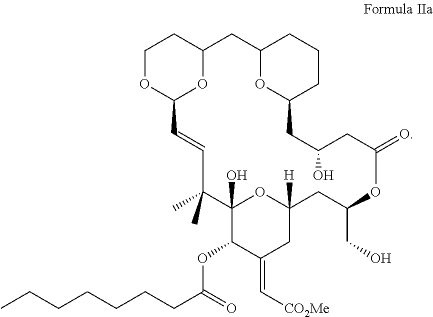

In another embodiment, the CABs are C26 des-methyl homologues of the native bryostatins, as illustrated in Formula VI:

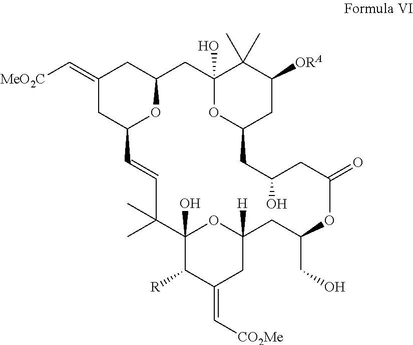

where OR^{A} and R^{B} correspond to the naturally occurring bryostatin substituents, including:

| Byrostatin | OR^{A} | R^{B} |
|---|---|---|
| 1 | —O₂C—CH₃ | —O₂C—CH═CH—CH═CH—CH₂—CH₂—CH₃ |
| 2 | —OH | —O₂C—CH═CH—CH═CH—CH₂—CH₂—CH₃ |
| 4 | —O₂C—C(CH₃C)₃ | —O₂C—CH₂—CH₃ |
| 5 | —O₂C—C(CH₃C)₃ | —O₂C—CH₃ |
| 6 | —O₂C—CH₂—CH₃ | —O₂C—CH₃ |
| 7 | —O₂C—CH₃ | —O₂C—CH₃ |
| 8 | —O₂C—CH₂—CH₃ | —O₂C—CH₂—CH₃ |
| 9 | —O₂C—CH₃ | —O₂C—CH₂—CH₃ |
| 10 | —O₂C—C(CH₃C)₃ | —H |
| 11 | —O₂C—CH₃ | —H |
| 12 | —O₂C—CH₂—CH₃ | —O₂C—CH═CH—CH═CH—CH₂—CH₂—CH₃ |
| 13 | —O₂C—CH₂—CH₃ | —H |
| 14 | —O₂C—C(CH₃C)₃ | —OH |
| 15 | —O₂C—CH₃ | —O₂C—CH═CH—CH═CH—CH₂—CH₂—CH₃ |
| 18 | —O₂C—C(CH₃C)₃ | —H |

such as C26 des-methyl Bryostatin 1, the compound of Formula VIa:

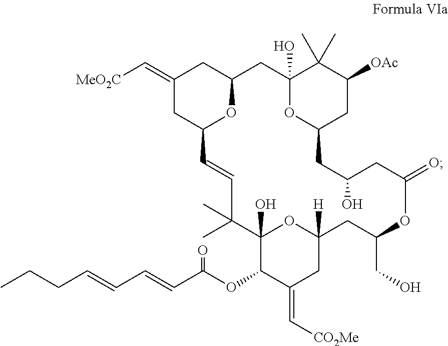

the C26 des-methyl homologues of the native bryostatins, as illustrated in Formula VII:

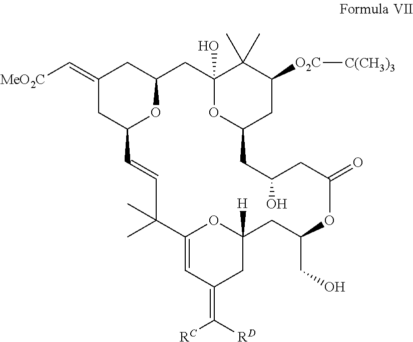

where R^{c} and R^{D} correspond to the naturally occurring bryostatin substituents, including:

| Byrostatin | R^{C} | R^{D} |
|---|---|---|
| 16 | —H | —CO₂Me |
| 17 | —CO₂Me | —H |

and to the C26 des-methyl homologues of the native Bryostatin 3, as illustrated in the following formula:

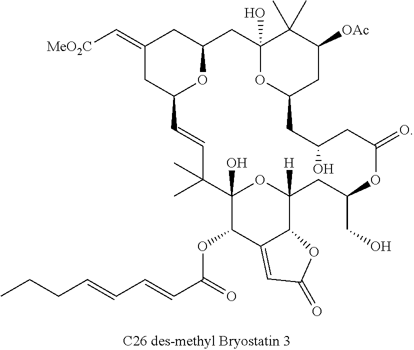

Further bryostatin analogs are disclosed in U.S. Pat. No. 7,256,286. In one embodiment, the bryostatin analogues are described in Formula I below:

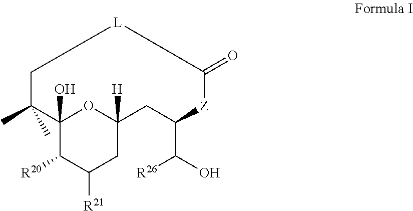

wherein: R²⁰ is H, OH, or -T-U—V—R′ where: T is selected from —O—, —S—, —N(H)— or —N(Me)-; U is absent or is selected from —C(O)—, —C(S)—, —S(O)— or —S(O)₂—; and V is absent or is selected from —O—, —S—, —N(H)— or —N(Me)-, provided that V is absent when U is absent; R²¹ is ═CR^{a}R^{b} or R²¹ represents independent moieties R^{c} and R^{d} where: R^{a} and R^{b} are independently H, CO₂R′, CONR^{c}R^{d} or R′; R^{c} and R^{d} are independently H, alkyl, alkenyl or alkynyl, or (CH₂)ₙCO₂R′ where n is 1, 2 or 3; R²⁶ is H, OH or R′; R′ (each instance) being independently selected from the group: H, alkyl, alkenyl or alkynyl, or aryl, heteroaryl, aralkyl or heteroaralkyl; L is a straight or branched linear, cyclic or polycyclic moiety, containing a continuous chain of preferably from 6 to 14 chain atoms, which substantially maintains the relative distance between the C1 and C17 atoms and the directionality of the C1C2 and C16C17 bonds of naturally-occurring bryostatin; and Z is —O— or —N(H)—; and the pharmaceutically acceptable salts thereof.

In one aspect, the recognition domain R²⁶ is H or methyl, particularly when R²¹ is ═C(H)CO₂R′ and/or where R²⁰ is —O₂CR′. Especially preferred are the compounds where R²⁶ is H. A preferred upper limit on carbon atoms in any of R^{d}, R^{e} and R′ is about 20, more preferably about 10 (except as otherwise specifically noted, for example, with reference to the embodiment of the invention where a preferred R²⁰ substituent has about 9 to 20 carbon atoms). In certain embodiments, R′ is a straight-chain alkyl, alkenyl (having from 1 to 6, preferably 1 to 4 double bonds, preferably trans double bonds) or alkynyl group. In a preferred aspect of the spacer domain of this embodiment, L contains a terminal carbon atom that, together with the carbon atom corresponding to C17 in the native bryostatin structure, forms a trans olefin. It is further preferred that L contain a hydroxyl on the carbon atom corresponding to C3 in the native bryostatin structure.

In another embodiment, the bryostatin analogues are represented by Formulae II-V:

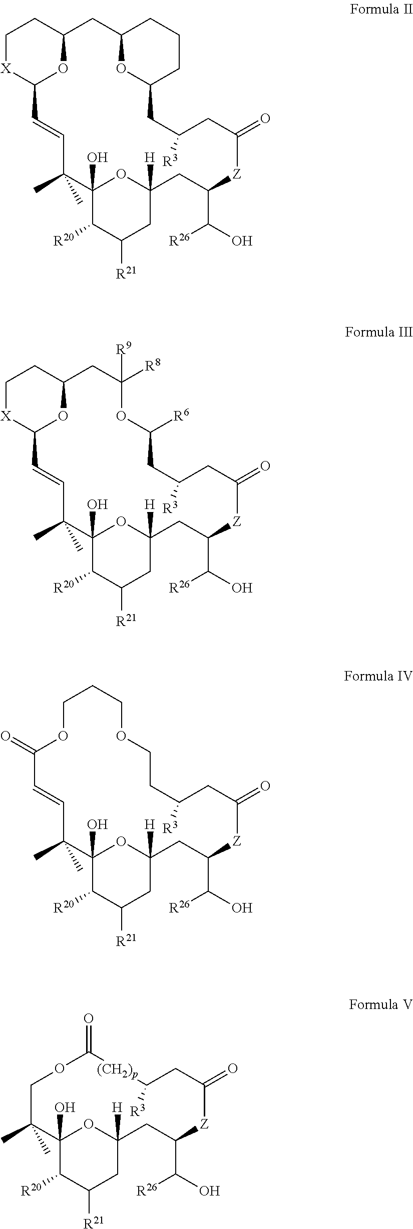

wherein: R³ is H, OH or a protecting group; R⁶ is H, H or ═O; R⁸ is selected from the group: H, OH, ═O, R′, —(CH₂)ₙO(O)CR′ or (CH₂)ₙCO₂-haloalkyl where n is 0, 1, 2, 3, 4 or 5, provided that R⁶ and R⁸ are not both ═O; R⁹ is H, OH or is absent; R²⁰, R²¹, R²⁶, R′ and Z are as defined above with respect to Formula I; p is 1, 2 or 3; and X is —CH₂—, —O—, —S— or —N(R^{e})— where R^{e} is COH, CO₂R′ or SO₂R′, X preferably being —O—, and the pharmaceutically acceptable salts thereof.

In yet another embodiment, the bryostatin analogues are represented by Formulae II-A to V-A:

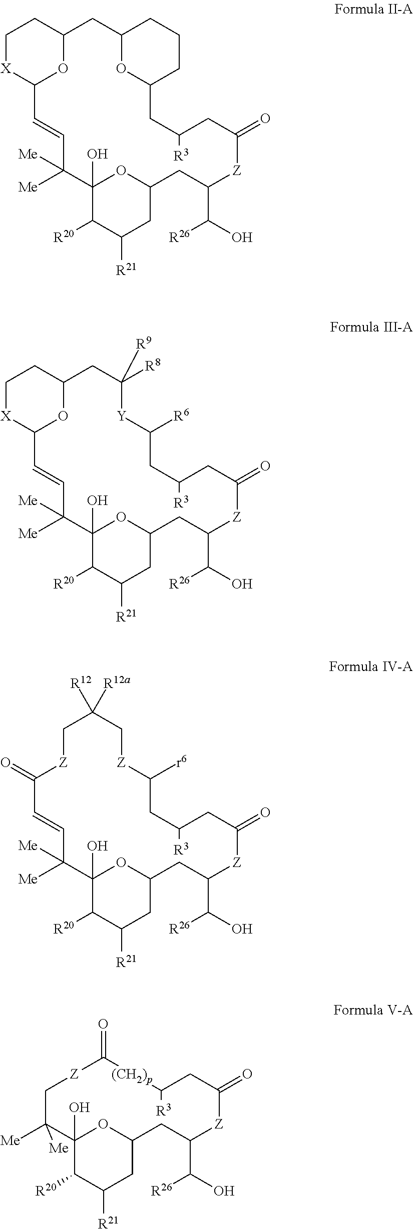

wherein: R³, R⁶, R⁹, R20, R²¹, R²⁶, R′, X, Z and p are as defined above with respect to Formulae II to V; R⁷ is absent or represents from 1 to 4 substituents on the ring to which it is attached, selected from lower alkyl, hydroxyl, amino, alkoxyl; alkylamino, ═O, acylamino, or acyloxy; R⁸ is as defined above including substituted or unsubstituted alkyl, alkenyl, alkynyl, aryl, heteroaryl, aralkyl, or heteroaralkyl, e.g., substituted with an alkoxy, acyloxy, acylamino, or alkylamino substituent, or, in Formula III-A when R⁶ represents H, H, then R⁸ and R⁹ taken together can represent ═O; R¹² and R^{12α} are independently for each occurrence, H, OH, lower alkyl, lower alkoxyl, or lower acyloxy, or R¹² and R^{12α} taken together represent ═O; and Y is CH₂, —O— or —N(H)—, and the pharmaceutically acceptable salts thereof. The compounds of Formulae II-A to V-A preferably have one or more of the stereochemical configurations respectively represented in Formulae II to V.

In one aspect of this embodiment, the CABs are C26 des-methyl analogues of Formula IIa:

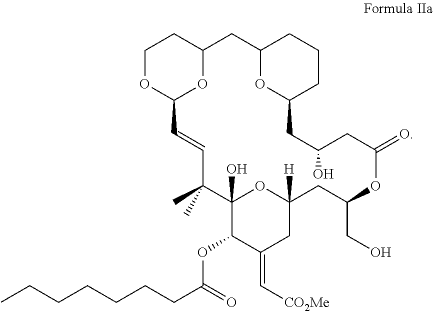

In another aspect of this embodiment, the CABs are C26 des-methyl homologues of the native bryostatins, as illustrated in Formula VI:

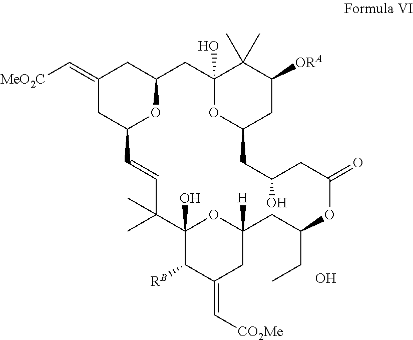

where R^{A} and R^{B} correspond to the naturally occurring bryostatin substituents, such as C26 des-methyl Bryostatin 1, the compound of Formula VIa:

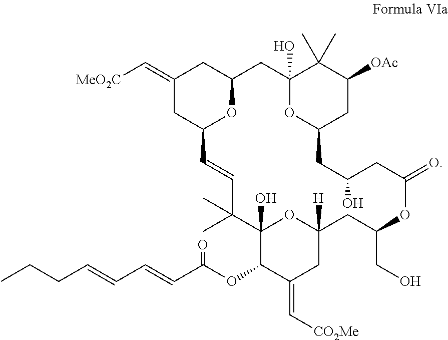 [SMILES: CCC/C=C/C=C/C(=O)O[C@H]1/C(=C/C(=O)OC)C[C@H]2C[C@H](CO)OC(=O)C[C@H](O)C[C@@H]3C[C@H](OC(C)=O)C(C)(C)[C@](O)(C[C@@H]4C/C(=C/C(=O)OC)C[C@H](/C=C/C(C)(C)[C@]1(O)O2)O4)O3]

Still further bryostatin analogues are disclosed U.S. Application No. 2010/0280262. In one embodiment, the CABs disclosed in the '262 publication have the structure of Formula I:

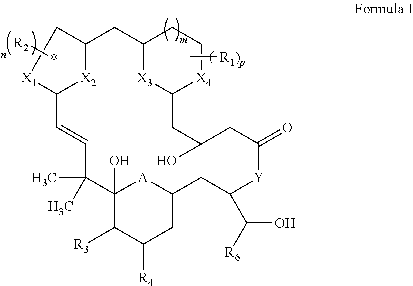

where R₁ and R₂ are independently H, —OH, —OR′, —NH₂, —NR′, ═CH₂, ═CHR′, ═O, —R′, halogen, —C(R)₂—COOR′, —C(R)₂—COO—C(R)₂—R′, —C(R)₂—COO—C(R)₂C═CR′, —(CH₂)_{q}O(O)CR′ or —(CH₂)_{q}CO₂-haloalkyl where q is 0, 1, 2, 3, 4 or 5, optionally substituted alkyl, optionally substituted alkenyl, optionally substituted alkynyl, optionally substituted alkoxy, optionally substituted alkyl amino, optionally substituted haloalkyl, optionally substituted haloalkoxy, optionally substituted alkylthio, optionally substituted aryl, optionally substituted heteroaryl, optionally substituted aralkyl, optionally substituted heteroaralkyl, optionally substituted heteroalkyl, optionally substituted cycloalkyl or optionally substituted cycloheteroalkyl, providing that valency is not violated;

R is H, optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl);

R₃ is independently H, —OH, or O(CO)R′;

R₄ is ═CR^{a}R^{b} or CHR^{c}R^{d}; R^{a} and R^{b} are independently H, —COOR′, —CONR^{c}R^{d} or R′; R^{c} and R^{d} are independently H, optionally substituted alkyl, optionally substituted alkenyl, optionally substituted alkynyl, (CH₂)ₜCONH₂R′, or (CH₂)ₜCOOR′ where t is 1, 2 or 3; R₆ is H, —OH, or R;

R′ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl), (CO)R″, or (COO)R″;

R″ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, or optionally substituted alkyl(cycloheteroalkyl);

A is C(R₁)₂, O, S, or N(R₁); the ring containing A is optionally partially unsaturated, provided that R₄ is not ═CR^{a}R^{b} when the ring carbon to which R₄ is attached is unsaturated; and

X₁, X₂, X₃, and X₄ are independently C(R₁)₂, O, S, or N(R₁); Y is O or N(R₁); m is 0 or 1; n is 0, 1, 2, or 3; p is 0, 1, 2, 3, or 4.

In one aspect of this embodiment, the compound does not have the structure of Formula A:

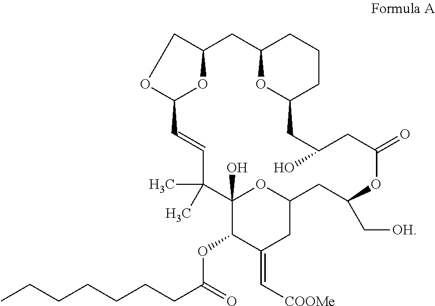 [SMILES: CCCCCCCC(=O)O[C@H]1/C(=C/C(=O)OC)CC2C[C@H](CO)OC(=O)C[C@H](O)C[C@@H]3CCC[C@H](C[C@@H]4CO[C@H](/C=C/C(C)(C)[C@]1(O)O2)O4)O3]

In one aspect of this embodiment, the compound of Formula I has the stereochemistry of Formula IA:

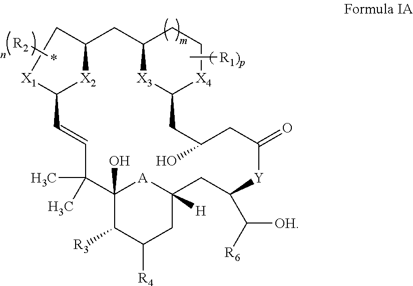

In a further aspect of this embodiment, the compound has the structure of Formula II:

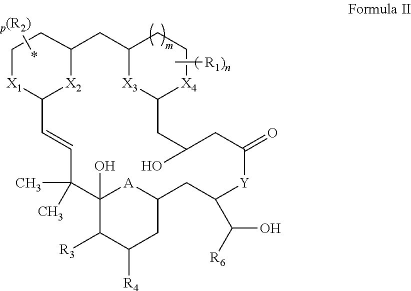

where R₁ and R₂ are independently H, —OH, —OR′, —NH₂, —NR′, ═CH₂, ═CHR′, ═O, —R′, halogen, —C(R)₂—COOR′, —C(R)₂—COO—C(R)₂—R′, —C(R)₂—COO—C(R)₂C═CR′, —(CH₂)_{q}O(O)CR′ or —(CH₂)_{q}CO₂-haloalkyl where q is 0, 1, 2, 3, 4 or 5, optionally substituted alkyl, optionally substituted alkenyl, optionally substituted alkynyl, optionally substituted alkoxy, optionally substituted alkyl amino, optionally substituted haloalkyl, optionally substituted haloalkoxy, optionally substituted alkylthio, optionally substituted aryl, optionally substituted heteroaryl, optionally substituted aralkyl, optionally substituted heteroaralkyl, optionally substituted heteroalkyl, optionally substituted cycloalkyl or optionally substituted cycloheteroalkyl, providing that valency is not violated;

R is H, optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl);

R₃ is independently H, —OH, or O(CO)R;

R₄ is ═CR^{a}R^{b} or CHR^{c}R^{d}; R^{a} and R^{b} are independently H, —COOR′, —CONR^{c}R^{d} or R′; R^{c} and R^{d} are independently H, optionally substituted alkyl, optionally substituted alkenyl, optionally substituted alkynyl, (CH₂)ₜCONH₂R′, or (CH₂)ₜCOOR′ where t is 1, 2 or 3;

R₆ is H, —OH, or R′;

R′ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl), (CO)R″, or (COO)R″;

R″ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, or optionally substituted alkyl(cycloheteroalkyl);

A is C(R₁)₂, O, S, or N(R₁); the ring containing A is optionally partially unsaturated, provided that R₄ is not ═CR^{a}R^{b} when the ring carbon to which R₄ is attached is unsaturated;

X₁, X₂, X₃, and X₄ are independently C(R₁)₂, O, S, or N(R₁); Y is O or N(R₁); and

n is 0, 1, 2 or 3; and p is 0, 1, 2, 3 or 4.

In one aspect of this embodiment, the compound of Formula II has the stereochemistry of Formula IIA:

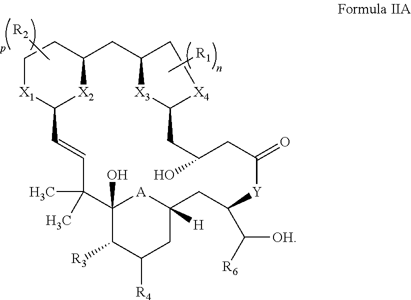

In another embodiment, the CABs are compounds of Formula III:

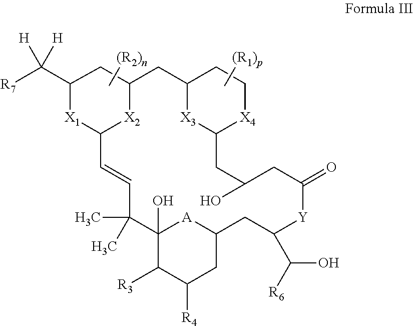

where R₁ and R₂ are independently H, —OH, —OR′, —NH₂, —NR′, ═CH₂, ═CHR′, ═O, —R′, halogen, —C(R)₂—COOR′, —C(R)₂—COO—C(R)₂—R′, —C(R)₂—COO—C(R)₂— C═CR′, —(CH₂)_{q}O(O)CR′ or —(CH₂)_{q}CO₂-haloalkyl where q is 0, 1, 2, 3, 4 or 5, optionally substituted alkyl, optionally substituted alkenyl, optionally substituted alkynyl, optionally substituted alkoxy, optionally substituted alkyl amino, optionally substituted haloalkyl, optionally substituted haloalkoxy, optionally substituted alkylthio, optionally substituted aryl, optionally substituted heteroaryl, optionally substituted aralkyl, optionally substituted heteroaralkyl, optionally substituted heteroalkyl, optionally substituted cycloalkyl, or optionally substituted cycloheteroalkyl, providing that valency is not violated;

R is H, optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl);

R₃ is independently H, —OH, or O(CO)R;

R₄ is ═CR^{a}R^{b} or CHR^{c}R^{d}; R^{a} and R^{b} are independently H, —COOR′, —CONR^{c}R^{d} or R; R^{c} and R^{d} are independently H, optionally substituted alkyl, optionally substituted alkenyl, optionally substituted alkynyl, (CH₂)ₜCONH₂R′, or (CH₂)ₜCOOR′ where t is 1, 2 or 3;

R₆ is H, —OH, or R; R₇ is H, —OH, —OR′, —NH₂, —NR′, —R′, halogen, —COOR′, —COOCH₂R′, —C(R)₂—COOCH₂C═CR′, —COCH₂R′, —C(R)₂—COCH₂C═CR′, optionally substituted alkyl, optionally substituted alkenyl, optionally substituted alkynyl, optionally substituted alkoxy, optionally substituted alkyl amino, optionally substituted haloalkyl, optionally substituted haloalkoxy, optionally substituted alkylthio, optionally substituted aryl, optionally substituted heteroaryl, optionally substituted alkylalkenyl, optionally substituted aralkyl, optionally substituted heteroaralkyl, optionally substituted heteroaralkylalkenyl, optionally substituted heteroalkyl, optionally substituted heteroalkylalkenyl, optionally substituted cycloalkyl, optionally substituted cycloheteroalkyl, or optionally substituted cycloheteroalkyl;

R′ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl), (CO)R″, or (COO)R″;

R″ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, or optionally substituted alkyl(cycloheteroalkyl);

A is C(R₁)₂, O, S, or N(R₁); the ring containing A is optionally partially unsaturated, provided that R₄ is not ═CR^{a}R^{b} when the ring carbon to which R₄ is attached is unsaturated; X₁, X₂, X₃, and X₄ are independently C(R₁)₂, O, S, or N(R₁); Y is O or N(R₁); and

n is 0 or 1; and p is 0, 1, 2, 3, or 4.

In one aspect of this embodiment, the compound of Formula III has the stereochemistry of Formula IIIA:

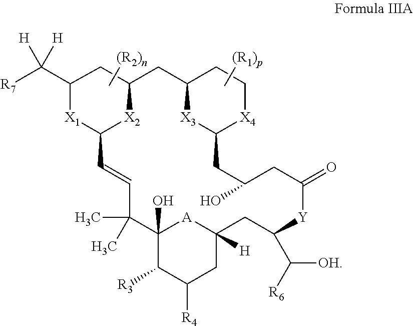

In another embodiment, the CABs are compounds of Formula IV:

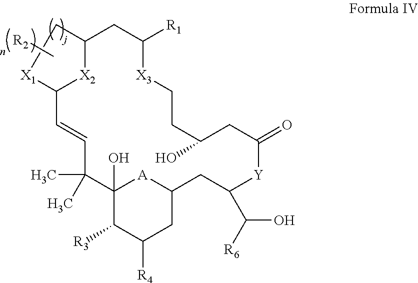

where R₁ and R₂ are independently H, —OH, —OR′, —NH₂, —NR′, ═CH₂, ═CHR′, ═O, —R′, halogen, —C(R)₂—COOR′, —C(R)₂—COO—C(R)₂—R′, C(R)₂—COO—C(R)₂C═CR′, —(CH₂)_{q}O(O)CR′ or —(CH₂)_{q}CO₂-haloalkyl where q is 0, 1, 2, 3, 4 or 5, optionally substituted alkyl, optionally substituted alkenyl, optionally substituted alkynyl, optionally substituted alkoxy, optionally substituted alkyl amino, optionally substituted haloalkyl, optionally substituted haloalkoxy, optionally substituted alkylthio, optionally substituted aryl, optionally substituted heteroaryl, optionally substituted aralkyl, optionally substituted heteroaralkyl, optionally substituted heteroalkyl, optionally substituted cycloalkyl or optionally substituted cycloheteroalkyl, providing that valency is not violated;

R is H, optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl);

R₃ is independently H, —OH, or O(CO)R;

R₄ is ═CR^{a}R^{b} or CHR^{c}R^{d}; R^{a} and R^{b} are independently H, —COOR′, CONR^{c}R^{d} or R′; R^{c} and R^{d} are independently H, optionally substituted alkyl, optionally substituted alkenyl, optionally substituted alkynyl, (CH₂)ₜCONH₂R′, or (CH₂)ₜCOOR′ where t is 1, 2 or 3;

R₆ is H, —OH, or R′;

R′ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl), (CO)R″, or (COO)R″;

R″ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, or optionally substituted alkyl(cycloheteroalkyl);

A is C(R₁)₂, O, S, or N(R₁); the ring containing A is optionally partially unsaturated, provided that R₄ is not ═CR^{a}R^{b}, when the ring carbon to which R₄ is attached is unsaturated;

X₁, X₂, and X₃, are independently C(R₁)₂, O, S, and N(R₁); Y is O or N(R₁); n is 0, 1, 2 or 3; and

j is 1 or 2, with the proviso that when j is 2, and X₁, X₂, and X₃ are all 0, then n is not 0.

In one aspect of this embodiment, the compound of Formula IV has the stereochemistry of Formula IVA:

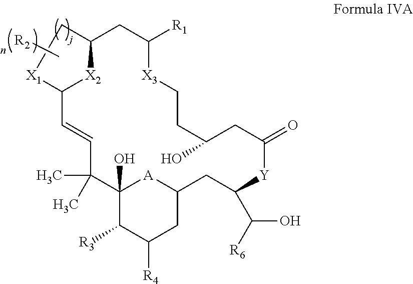

In another embodiment, the CABs are compounds of Formula V or Formula VI:

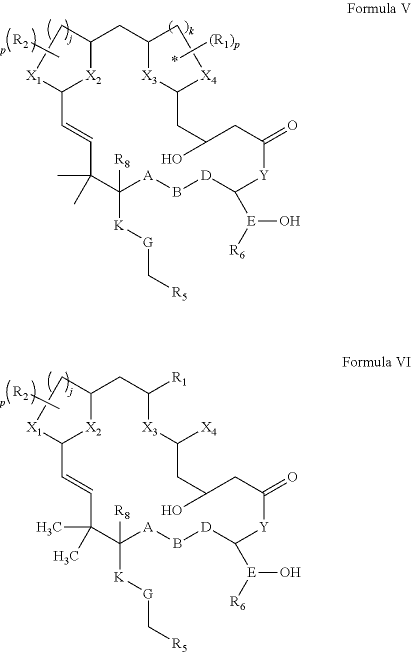

wherein R₁, R₂, and R₅ are independently H, —OH, —OH′, —NH₂, —NR′, ═CH₂, ═CHR′, ═O, —R′, halogen, —C(R)₂—COOR′, —C(R)₂—COO—C—R′, —C(R)₂—COO—C(R)₂—C═CR′, —(CH₂)_{q} O(O)CR′ or —(CH₂)_{q}CO₂-haloalkyl where q is 0, 1, 2, 3, 4 or 5 optionally substituted alkyl, optionally substituted alkenyl, optionally substituted alkynyl, optionally substituted alkoxy, optionally substituted alkyl amino, optionally substituted haloalkyl, optionally substituted haloalkoxy, optionally substituted alkylthio, optionally substituted aryl, optionally substituted heteroaryl, optionally substituted aralkyl, optionally substituted heteroaralkyl, optionally substituted heteroalkyl, optionally substituted cycloalkyl or optionally substituted cycloheteroalkyl, providing that valency is not violated;

R is H, optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl);

R₆ is independently H, —OH or R;

R₈ is H, OH, or R; R′ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, optionally substituted alkyl(cycloheteroalkyl), (CO)R″, or (COO)R″;

R″ is optionally substituted alkyl, optionally substituted alkenyl, optionally substituted alkynyl, optionally substituted aryl, optionally substituted heteroaryl, optionally substituted aralkyl, optionally substituted heteroaralkyl, optionally substituted heteroalkyl, or optionally substituted alkyl(cycloheteroalkyl);

A is C(R₁)₂, O, S, or N(R₁);

X₁, X₂, X₃, and X₄ are independently C(R₁)₂, O, S, and N(R₁);

Y is O or N(R₁); j and k are independently 1 or 2; p is independently for each ring to be 0, 1, 2, or 3;

B, D, E, G, and K are independently CR^{a}R^{b}, C═O, H, O, S, or NR′, where R^{a} and R^{b} are independently H, —COOR′, —CONR^{c}R^{d} or R; R^{c} and R^{d} are independently H, alkyl, alkenyl or alkynyl, or (CH₂)ₜCOOR′ where t is 1, 2 or 3;

optionally A is linked with K or G to form a substituted or unsubstituted monocyclic or bicyclic ring of 5-10 members having 0, 1, 2, 3, or 4 heteroatoms; optionally B is linked with K or G to form a substituted or unsubstituted monocyclic or bicyclic ring of 5-10 members having 0, 1, 2, 3, or 4 heteroatoms; optionally D is linked with K or G to form a substituted or unsubstituted monocyclic or bicyclic ring of 5-10 members having 0, 1, 2, 3, or 4 heteroatoms; optionally E is linked with B, D, K, or G to form a substituted or unsubstituted monocyclic or bicyclic ring of 5-10 members having 0, 1, 2, 3, or 4 heteroatoms; optionally E is linked with B and G or D and K to form a substituted or unsubstituted bicyclic ring of 7-14 members having 0, 1, 2, 3, or 4 heteroatoms; optionally K is linked with B and E to form a substituted or unsubstituted bicyclic ring of 7-14 members having 0, 1, 2, 3, or 4 heteroatoms; its pharmaceutically acceptable salts and esters thereof; wherein any of the rings formed by linking A, B, D, E, G and/or K may be saturated, unsaturated or aromatic; and wherein the linker linking any of the groups A, B, D, E, K or G comprises two to seven C(R)₂ groups, and each C(R)₂ group may be optionally substituted by a hetero atom, or a —C(O)— group.

In one aspect of this embodiment, the compound of Formula V has the stereochemistry of Formula VA.

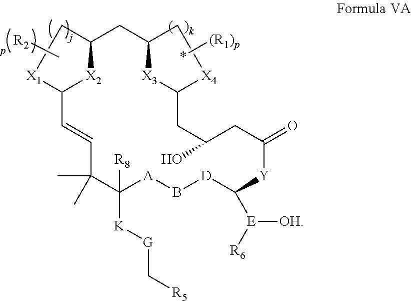

In another aspect of this embodiment, the compound of Formula VI has the stereochemistry of Formula VIA:

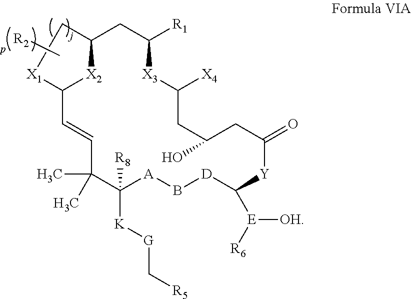

Further bryostatin analogs are disclosed in U.S. Publication No. 20090270492. In one embodiment, these analogues are represented by Formula I:

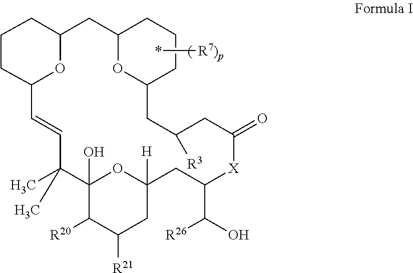

wherein X is O, S or NR′, R³ is H or OH; R⁷ is selected from the group consisting of optionally substituted lower alkyl, optionally substituted alkenyl, hydroxyl, amino, optionally substituted alkylamino, ═O, optionally substituted acylamino, OC(O)NR′R′, OC(O)OR′, OC(O)R′, and substituted acyloxy; p is 0, 1, 2, 3, or 4;

R²⁰ is H, OH, or -T-U—V—R′ where T is selected from —O—, —S —, —N(H)— or —N(Me)-; U is absent or is selected from —C(O)—, —C(S)—, —S(O)— or —S(O)₂—; and V is absent or is selected from —O—, —S—, —N(H)— or —N(Me)-, provided that V is absent when U is absent;

R²¹ is ═CR^{a}RFb or R²¹ represents independent moieties R^{c} and R^{d} where R^{a} and R^{b} are independently H, CO₂R′, CONR^{c}R^{d} or R′; R^{c} and R^{d} are independently H, alkyl, alkenyl or alkynyl, or (CH₂)ₙCO₂R′ where n is 1, 2 or 3;

R²⁶ is H or R′; and R′ is independently selected from the group consisting of H, optionally substituted alkyl, optionally substituted alkenyl, optionally substituted alkynyl, optionally substituted aryl, optionally substituted heteroaryl, optionally substituted aralkyl and optionally substituted heteroaralkyl, and their pharmaceutically acceptable salts thereof.

In one aspect of this embodiment, the compound is not

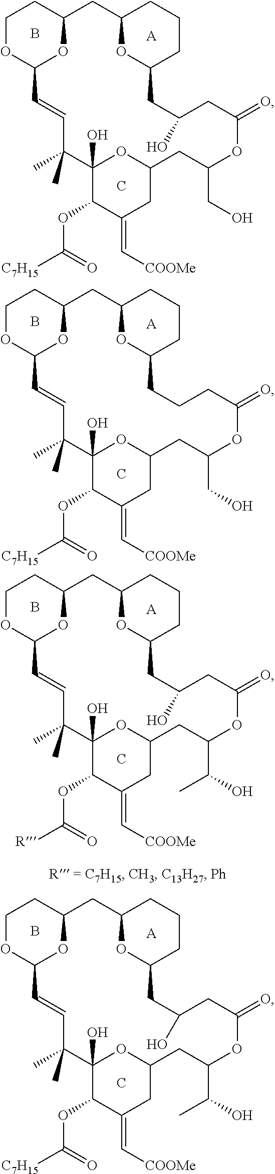

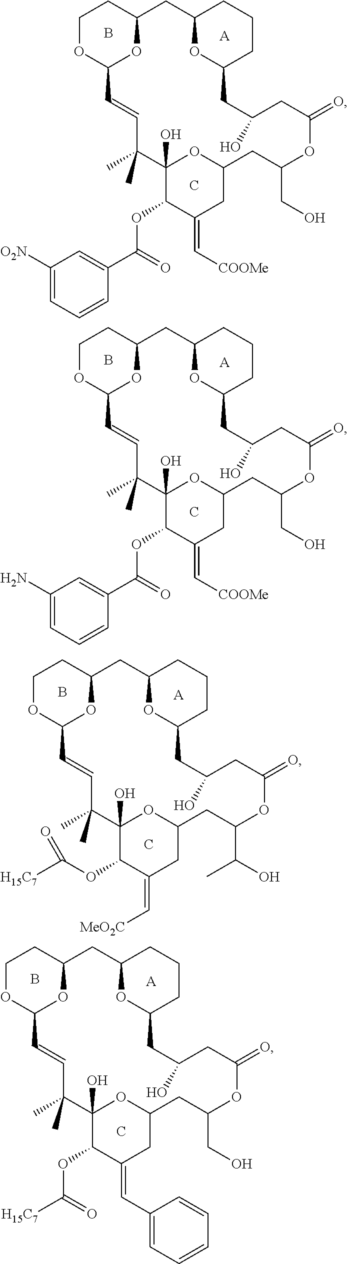

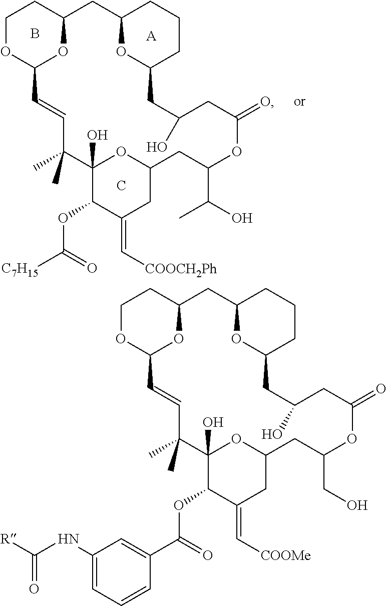

wherein R″ is selected from the group consisting of CH₃, Ph, C₁₃H₂₇, C₇H₁₅, CH₂₀(CH₂)₂O(CH₂)₂OCH₃, and

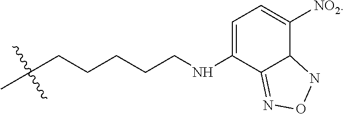 [SMILES: *CCCCCNC1=CC=C([N+](=O)[O-])C2NON=C12]

In one aspect of this embodiment, the compound of Formula I has the stereochemistry of Formula IA:

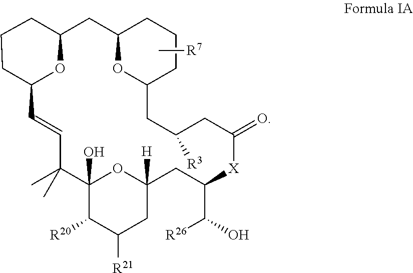

In some embodiments of the invention, a compound of Formula I is provided wherein X is O. In some embodiments of the invention, a compound of Formula I is provided wherein R³ is OH. In other embodiments, a compound of Formula I is provided wherein R²⁶ is H.

In another embodiment, the CAB is a compound of Formula II:

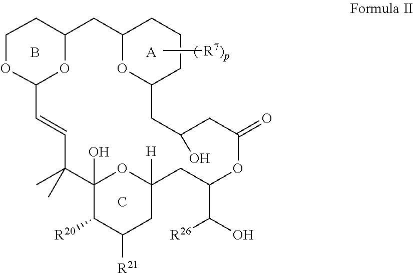

wherein R⁷ is selected from the group consisting of optionally substituted lower alkyl, optionally substituted alkenyl, hydroxyl, amino, optionally substituted alkylamino, ═O, optionally substituted acylamino, OC(O)NR′R′, OC(O)OR′, OC(O)R′, and substituted acyloxy; p is 0, 1, 2, 3, or 4;

R²⁰ is H, OH, or -T-U—V—R′ where T is selected from —O—, —S—, —N(H)— or —N(Me)-; U is absent or is selected from —C(O)—, —C(S)—, —S(O)— or —S(O)₂—; and V is absent or is selected from —O—, —S—, —N(H)— or —N(Me)-, provided that V is absent when U is absent;

R²¹ is ═CR^{a}R^{b} or R²¹ represents independent moieties R^{c} and R^{d} where R^{a} and R^{b} are independently H, CO₂R′, CONR^{c}R^{d} or R′; R^{c} and R^{d} are independently H, alkyl, alkenyl or alkynyl, or (CH₂)ₙCO₂R′ where n is 1, 2 or 3;

R²⁶ is H or R′; and R′ is independently selected from the group consisting of H, optionally substituted alkyl, optionally substituted alkenyl, optionally substituted alkynyl, optionally substituted aryl, optionally substituted heteroaryl, optionally substituted aralkyl, optionally substituted aralkenyl, and optionally substituted heteroaralkyl, and their pharmaceutically acceptable salts thereof.

In one aspect of this embodiment, the compound is not:

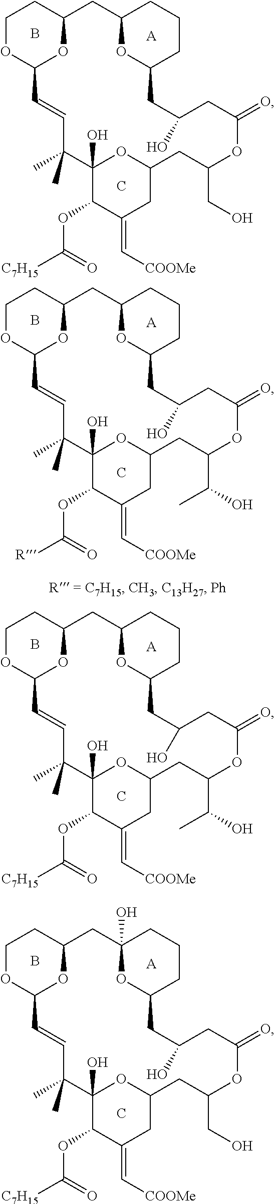

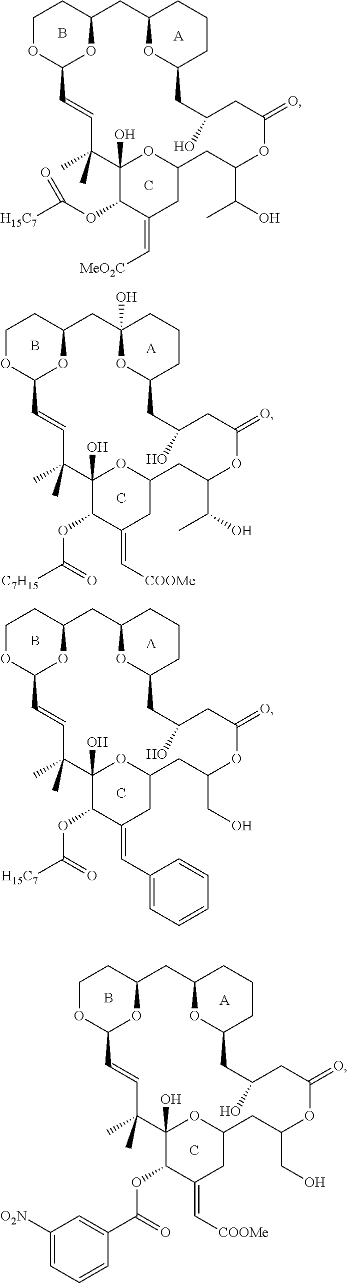

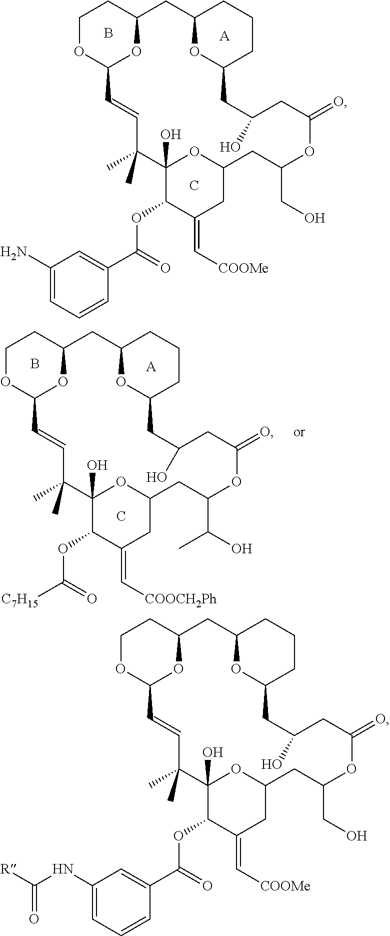

wherein R″ is selected from the group consisting of CH₃, Ph, C₁₃H₂₇, C₇H¹⁵, CH₂O(CH₂)₂O(CH₂)₂OCH₃, and

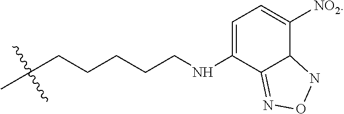 [SMILES: *CCCCCNC1=CC=C([N+](=O)[O-])C2NON=C12]

In one aspect of this embodiment, the compound of Formula II has stereochemistry of Formula IIA:

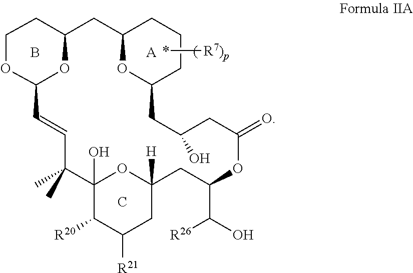

In one aspect of this embodiment, the compound of Formula II is provided wherein R26 is H.

In another embodiment, the CABs are compounds of Formula III:

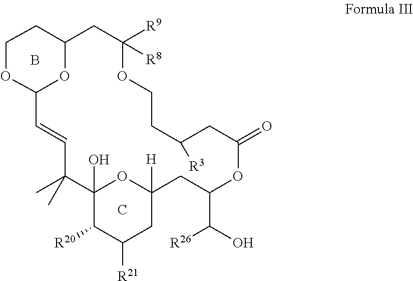

wherein R³ is H or OH; R⁸ is selected from the group consisting of H, OH, R′, —(CH₂)O₂CR′, and —(CH₂)ₙO₂C-haloalkyl; n is 0, 1, 2, 3, 4, or 5;

R⁹ is H or OH;

R²⁰ is H, OH, or -T-U—V—R′ where T is selected from —O—, —S—, —N(H)— or —N(Me)-; U is absent or is selected from —C(O)—, —C(S)—, —S(O)— or —S(O)₂—; and

V is absent or is selected from —O—, —S—, —N(H)— or —N(Me)-, provided that V is absent when U is absent;

R²¹ is ═CR^{a}R^{b} or R²¹ represents independent moieties R^{c} and R^{d} where R^{a} and R^{b} are independently H, CO₂R′, CONR^{c}R^{d} or R′; R^{c} and R^{d} are independently H, alkyl, alkenyl or alkynyl, or (CH₂)ₙCO₂R′ where n is 1, 2 or 3;

R²⁶ is H or R′; and R′ is independently selected from the group consisting of H, optionally substituted alkyl, optionally substituted alkenyl, optionally substituted alkynyl, optionally substituted aryl, optionally substituted heteroaryl, optionally substituted aralkyl, optionally substituted aralkenyl, and optionally substituted heteroaralkyl, and their pharmaceutically acceptable salts thereof.

In one aspect of this embodiment, the compound is not:

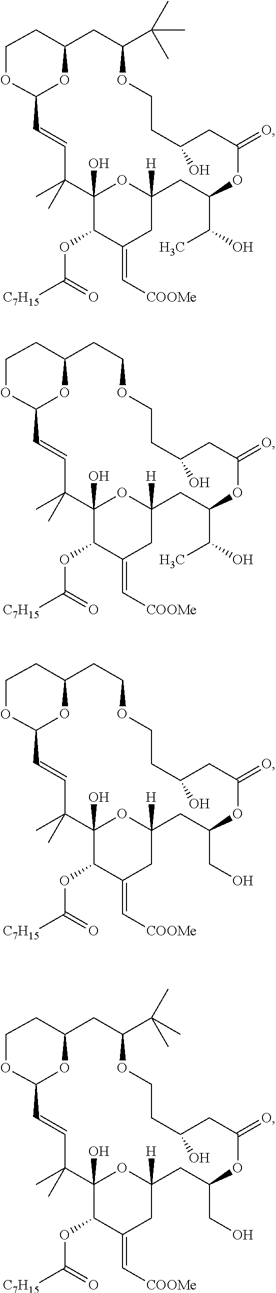

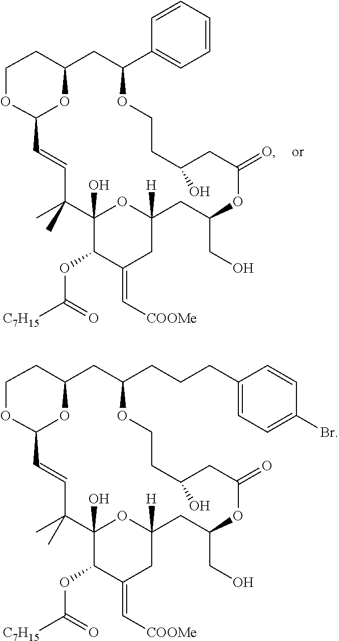

In one aspect of this embodiment, a compound of Formula II is provided having the stereochemical configuration of Formula IIIA:

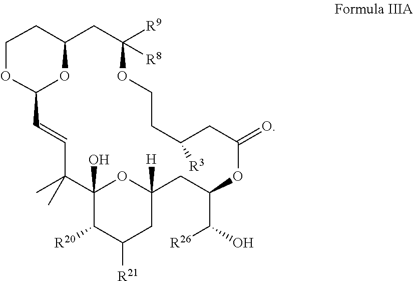

In another embodiment, the CAB is a compound of Formula IV:

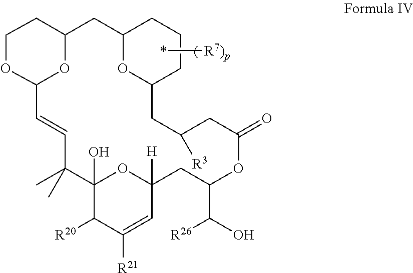

wherein R³ is H or OH;

R⁷ is selected from the group consisting of optionally substituted lower alkyl, optionally substituted alkenyl, hydroxyl, amino, optionally substituted alkylamino, ═O, optionally substituted acylamino, OC(O)NR′R′, OC(O)OR′, OC(O)R′, and substituted acyloxy; p is 0, 1, 2, 3, or 4;

R²⁰ is H, OH, or -T-U—V—R′ where T is selected from —O—, —S—, —N(H)— or —N(Me)—; U is absent or is selected from —C(O)—, —C(S)—, —S(O)— or —S(O)₂—; and V is absent or is selected from —O—, —S—, —N(H)— or —N(Me)—, provided that V is absent when U is absent;

R²¹ is H, alkyl, alkenyl or alkynyl, or (CH₂)ₙCO₂R′ where n is 1, 2 or 3;

R²⁶ is H or R′; and R′ is independently selected from the group consisting of H, optionally substituted alkyl, optionally substituted alkenyl, optionally substituted alkynyl, optionally substituted aryl, optionally substituted heteroaryl, optionally substituted aralkyl, optionally substituted aralkenyl, and optionally substituted heteroaralkyl, and their pharmaceutically acceptable salts thereof.

In one aspect of this embodiment, the compound of Formula IV has the stereochemistry of Formula IVA:

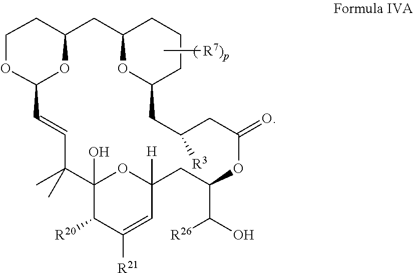

Histone Deacetylase Inhibitors (HDACi)

The present invention uses a combination of prostratin or analogs thereof with a histone deacetylase inhibitor (HDAC inhibitor) and HAART.

One limitation associated with treating HIV-1 is that while it is not fully understood how HIV-1 evades the immune response and establishes latency in resting cells, it is believed that a variety of signaling molecules and transcription factors appear to play a role, and thus offer potential targets for intervention. Thus, histone deacetylase (HDAC) inhibitors are used to confer reactivation by up regulation of pro-HIV-1 genes, effectively coaxing virus out from previously resting cells.

While not wishing to be bound to a particular theory, it is believed that the HDACi increases prostratin (or prostratin analog)-induced DNA-binding activity of nuclear NF-κB and degradation of cytoplasmic NF-κB inhibitor, IκBα. It is believed that the combined treatment (prostratin or analog thereof plus HDACi) results in a more pronounced nucleosomal remodeling in the U1 viral promoter region than using either compound alone. In this manner, latent HIV-1 is eradicated.

One example of a reactivation agent that could be used in this manner is panobinostate, which is described, for example, in Lewin, et al., “HIV cure and eradication: how will we get from the laboratory to effective clinical trials?” AIDS:24 Apr. 2011.

Representative HDAC inhibitors include butyric acid (including sodium butyrate and other salt forms), Valproic acid (including Mg valproate and other salt forms), suberoylanilide hydroxamic acid (SAHA), Vorinostat, Romidepsin (trade name Istodax), Panobinostat (LBH589), Belinostat (PXD101), Mocetinostat (MGCD0103), PCI-24781, Entinostat (MS-275), SB939, Resminostat (4SC-201), Givinostat (ITF2357), CUDC-101, AR-42, CHR-2845, CHR-3996, 4SC-202, sulforaphane, BML-210, M344, CI-994; CI-994 (Tacedinaline); BML-210; M344; MGCD0103 (Mocetinostat); and Tubastatin A. Additional HDAC inhibitors are described in U.S. Pat. No. 7,399,787.

Pharmaceutical Compositions

Administration of the active compounds (i.e., HAART/ARV, with prostratin or analogs thereof and HDACi cycled periodically) described herein can be via any of the accepted modes of administration for therapeutic agents. These methods include oral, parenteral, transdermal, subcutaneous and other systemic modes. In some instances it may be necessary to administer the composition parenterally.

Depending on the intended mode, the compositions may be in the form of solid, semi-solid or liquid dosage forms, such as, for example, tablets, suppositories, pills, capsules, powders, liquids, suspensions, skin patch, or the like, preferably in unit dosage forms suitable for single administration of precise dosages. The compositions will include a conventional pharmaceutical excipient and an active compound of formula I or the pharmaceutically acceptable salts thereof and, in addition, may include other medicinal agents, pharmaceutical agents, carriers, adjuvants, diluents, etc.

The amount of active compound administered will, of course, be dependent on the subject being treated, the severity of the affliction, the manner of administration and the judgment of the prescribing physician. However, an effective dosage is in the range of 0.001-100 mg/kg/day, preferably 0.005-5 mg/kg/day. For an average 70 kg human, this would amount to 0.007-7000 mg per day, or preferably 0.05-350 mg/day. Alternatively, the administration of compounds as described by L. C. Fritz et al. in U.S. Pat. No. 6,200,969 is followed. One of skill in the art with this disclosure can create an effective pharmaceutical formulation.

For solid compositions, conventional non-toxic solid include, for example, pharmaceutical grades of mannitol, lactose, starch, magnesium stearate, sodium saccharin, talcum, cellulose, glucose, sucrose, magnesium carbonate, and the like may be used. The active compound as defined above may be formulated as suppositories using, for example, polyalkylene glycols, for example, propylene glycol, as the carrier. Liquid pharmaceutically administrable compositions can, for example, be prepared by dissolving, dispersing, etc. an active compound as defined above and optional pharmaceutical adjuvants in a excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 17th Edition, 1985. The composition or formulation to be administered will, in any event, contain a quantity of the active compound(s), a therapeutically effective amount, i.e., in an amount effective to alleviate the symptoms of the subject being treated.

For oral administration, a pharmaceutically acceptable non-toxic composition is formed by the incorporation of any of the normally employed excipients, such as, for example pharmaceutical grades of mannitol, lactose, starch, magnesium stearate, sodium saccharin, talcum, cellulose, glucose, sucrose, magnesium, carbonate, and the like. Such compositions take the form of solutions, suspensions, tablets, pills, capsules, powders, sustained release formulations and the like. Such compositions may contain 10%-95% active ingredient, preferably 1-70%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, etc.

A more recently devised approach for parenteral administration employs the implantation or skin patch for a slow-release or sustained-release system, such that a constant level of dosage is maintained. See. e.g., U.S. Pat. No. 3,710,795, which is incorporated herein by reference.

Methods of Treatment

In one embodiment, the methods described herein can be used to purge and eradicate the HIV-1 virus from a patient's system, particularly in the latent cells in which HIV-1 resides. The bulk of viral load is eradicated from the majority of cells using conventional antiretroviral (ARV) therapy, such as a HAART therapy, as described above. As discussed above, it is desirable that the ARV therapy include at least one integrase inhibitor, at least one entry inhibitor, such as a CCR5 antagonist, and at least one, and preferably two, reverse transcriptase inhibitors. The antiretroviral (ARV) regimen is used until viral loads are reduced to extremely low levels, preferably an undetectable viral load (<50 copies/mL).

The ARV regimen typically includes agents which interrupt the viral lifecycle prior to viral DNA integration into the host cell genome. For this reason, it is desired that the regimen includes at least one integrase inhibitor at least one entry inhibitor, in addition to reverse transcriptase inhibitors. These compounds can help prevent additional cells from becoming infected as the virus is being driven out of latent cells.

To access and eliminate the HIV residing in latent cells, compounds are administered that encourage viral production in the latent cells, preferably without activating those cells, while maintaining the ARV therapy.

It is important to know when to administer each therapy, and this typically involves a series of cycles, where a first cycle involves the ARV therapy, and the next cycle involves both ARV therapy and administration of compounds that encourage viral production in the latent cells. The cycles are repeated for a sufficient number of times that substantial, if not complete, viral eradication is achieved. In one aspect of this method, the administration of each therapy (ARV alone, or ARV plus compounds which induce viral transcription) is based on a time-based schedule.

Ideally, the specific antiretroviral regimen includes multiple ARVs that are known to be effective in biological compartments outside of the lymph and serum, particularly the central nervous system, as not all drugs penetrate the CNS equally. In one aspect of this embodiment, the ARV regimen includes raltegravir, maraviroc, abacavir, and zidovudine (AZT), where elvitegravir or dolutegravir (assuming FDA approval) can be substituted for raltegravir.

The set of compounds that provoke viral replication in latently infected cells can include a mixture of compounds, some of which are already FDA approved and could be repurposed. These compounds can be used in conjunction with the ARVs as adjuvant therapy. That is, while the ARVs limit viral replication and integration, the adjuvants encourage latent cell depletion via viral production.

In one embodiment, the compounds that provoke viral replication in latently infected cells include at least one of the chemical analogues of prostratin (CAPs) (Stanford University), sodium butyrate, an HDAC inhibitor, and a drug typically approved for T-cell lymphomas, such as romidepsin or vorinostat (both are also HDAC inhibitors).

Using both the ARVs and the adjuvants, cycling the administration of the adjuvants, ideally according to a treatment schedule, encourages latent viral production, thereby killing the cells harboring latent virus, and preventing new cellular infection. By killing off more cells per round of therapy than are being infected, the virus can eventually be purged from the body.

In one embodiment, where a schedule is used to determine when to administer each therapy, the schedule for using both the ARVs and the adjuvants is as follows:

From time point zero, from when a patient is identified as a candidate for eradication therapy, the patient is put on a daily ARV regimen alone for approximately 6 months. Candidates for ARV therapy includes those who are recently infected and that are still naïve to ARVs, as well any patient for whom resistance testing indicates that an appropriate ARV regimen can be assembled such that all agents are active against any mutations present in the patients particular strain of HIV-1.

Where a CCR5 antagonist is used to prevent entry of the virus into cells (i.e., as an entry inhibitor), ideal candidates have an R5 tropic virus. This can be determined by a clinically validated tropism assay, and by ensuring that the virus is an R5 tropic virus, one can ensure that the CCR5 antagonist component of the ARV regimen is effective.

At around 6 months, provided that the patient has an undetectable viral load (<50 copies/mL), the patient would continue with the ARV regimen and would also begin taking the adjuvant therapy (i.e., CAP(s) and an HDACi).

The patient is treated, in cycles, with a second HDAC inhibitor, such as romidepsin or vorinostat, the dosage is adjusted so that it is appropriate for use in treating HIV-1 rather than for treating cancer. Those of skill in the art know the dosage ranges for these compounds when used to treat HIV-1.

Ideally, the treating physician periodically monitors the patient's viral load. Such monitoring can be, for example, every few weeks, such as around every two weeks, until the patient's viral load returns to undetectable levels (<50 copies/mL). At that point, the doctor can administer another dose of the second HDAC inhibitor, which can be romidepsin or vorinostat.

In one embodiment, the cycle of administration of romidepsin or vorinostat and viral load monitoring is continued for 7 rounds, 8 to 10 rounds, or up to 20 rounds. A schematic illustration of this embodiment is shown in FIG. 8.

In another embodiment, at approximately 6 months, provided that the patient has an undetectable viral load (<50 copies/mL), the patient would continue with the ARV regimen and would begin cycling in adjuvant therapy using prostratin or one or more CAP(s), along with one or more HDAC inhibitors, such as one or more of sodium butyrate, romidepsin and vorinostat. Where romidepsin or vorinostat are administered, an adjusted dose (rather than the one given for oncology) of romidepsin or vorinostat is used.

The treating physician would then periodically monitor the patient's viral load, for example, every 2 weeks, until the patient's viral load returns to undetectable levels (<50 copies/mL). At that point, the doctor would administer another dose of the adjuvant therapy.

In one aspect of this embodiment, the cycle of administration of adjuvant therapy and viral load monitoring is continued for 7 rounds, 8 to 10 rounds, or up to 20 rounds.

In one aspect of this embodiment, romidpesin or vorinostat is used, along with sodium butyrate and prostratin or a CAP, at these intervals.

In either embodiment, at the end of seven rounds of therapy, patients can be checked by a single-copy VL assay to determine viral persistence. If virus is detected, additional cycles of therapy are recommended, for example, three more cycles of therapy. If virus is not detected, then patients can be taken off all drugs, including the ARVs, and periodically monitored with viral load tests. Such periodic monitoring can be, for example, every few weeks, such as every two or three weeks.

In either embodiment, the cycling of adjuvant therapy can be a single dosage, or multiple doses over a period of one day, two days, three days, four days, five days, six days, seven days, up to two weeks, up to three weeks, or up to one month. In one aspect of this embodiment, each cycle of adjuvant therapy is administered for a time between one week and ten weeks.

Patients with persistent absence of VL detection after three months can then be monitored less frequently for up to a year for re-emergence of virus. If no re-emergence is detected, then the virus has likely been purged from the patient's system.

If re-emergence is detected at any point, then the therapy may not work for such a patient. It is anticipated that not all patients will be successful with this adjuvant approach.

Co-Administration of Targeted Cytotoxic Agents

In HIV infection, chronically infected cells form a reservoir of HIV infection that has proved impossible to eliminate even after long periods of viral suppression using antiretroviral therapy. Cells harboring drug-resistant virus and viral DNA can fuel viral rebound in treatment-experienced patients. The methods described above can be used to activate viral production within quiescent HIV harbor cells, thereby expose these resulting cells. However, in one embodiment, the methods further involve the additional step of providing targeted cytotoxic agents, such as radio-immunotherapy (RIT), to kill those cells which have been induced to produce HIV.

As stated above, achieving eradication requires three components:

1) Administration of an antiretroviral (ARV) regimen to reduce viral loads to virtually undetectable levels,

2) Administration of therapeutics that induce viral replication in harboring cells, ideally without activating the cell; and

3) Targeted killing of cells producing viral particles.

The likelihood of success of these three steps can be increased by using a time-based algorithm that dictates when both the ARVs and the novel therapeutics are given.

As described above, the ARV regimen is ideally selected from the set of available FDA approved ARVs that interrupt the viral lifecycle prior to viral DNA integration into the host cell genome. In one aspect, the regimen includes at least one integrase inhibitor, one entry inhibitor, likely a CCR5 antagonist, and at least one, and preferably two, reverse transcriptase inhibitors. In one aspect of this embodiment, the antiretroviral regimen includes multiple ARVs that are known to be effective in biological compartments outside of the lymph and serum, particularly the central nervous system, as not all drugs penetrate the CNS equally. Representative examples of FDA approved ARVs include raltegravir or elvitegravir, maraviroc, abacavir, and zidovudine (AZT). An additional integrase inhibitor that may be substituted for raltegravir, if it is approved for use, is dolutegravir.

The compound(s) used to induce replication of latent virus in harboring cells, or viral replication agents (VRAs), will be prostratin, bryostatin-1, or one of the analogues of these compounds. Prostratin, bryostatin-1, and their analogues have demonstrated the ability to induce latent viral replication without encouraging cell cycle progression by manipulating the PKC pathway(s) within the cell. In one aspect of this embodiment, one or more histone deacetylase inhibitors (HDACis), such as vorinostat, sodium butyrate, valproic acid, romidepsin, and the like is used in combination with the VRAs.

Once quiescent cells are induced to produce virus, the HIV-producing cells can be killed by administering a targeted cytotoxic agent, such as a radioactive-labeled antibody.

The targeted cytotoxic agent can be administered before, after, or concurrently with the agents, such as the bryostatin, prostratin, and analogs thereof, which induce the quiescent cells to produce virus. In one embodiment, the targeted cytotoxic agent is administered after the agents which induce the quiescent cells to produce virus, and in another embodiment, the targeted cytotoxic agent is administered concurrently with the agents which induce the quiescent cells to produce virus. In one aspect of this embodiment, the targeted cytotoxic agent is an antibody, protein, or peptide, and is administered via intravenous infusion, and the agent which induces the quiescent cells to produce virus is administered in the same infusion.

Known as radioimmunotherapy (RIT), the antibodies bind specifically and kill cells that have produced viral particles. RIT is disclosed, for example, in Dadachova et al, (2006) Targeted killing of virally infected cells by radiolabeled antibodies to viral proteins. PLoS Med 3(11): e427, Wang, et al., (2007) Treating cancer as an infectious disease—viral antigens as novel targets for treatment and potential prevention of tumors of viral etiology. PLOS One 2(10): e1114, Dadachova and Casadevall, (2009) Radioimmunotherapy of infectious diseases. Semin Nucl Med. 39(2):146-53, and Dadachova et al. Targeted killing of virally infected cells by radiolabelled antibodies to viral proteins. PloS Medicine 3 (11): e427, 2006.

RIT takes advantage of the fact that each type of antibody is programmed to seek out just one type of antigen in the body. By attaching radioactive material to a particular antibody, radiation can be targeted at specific cells that express the corresponding antigen, minimizing collateral damage to other tissues. Because of this targeting, the antibodies will kill infected cells that have been stimulated to produce virus, but will not kill a significant number of cells that are not infected with HIV. The RIT is not used to target virus particles, per se, but rather, lymphocytes that harbor the virus. Lymphocytes are extremely radiosensitive, so are easily killed using this approach.

Radiolabeled antibodies kill HIV infected human cells through binding to viral antigens on these cells. Suitable viral antigens include, but are not limited to, HIV's gp120 and gp41 envelope proteins. Suitable radiolabels include bismuth 213 and rhenium 188, which are preferred for their relatively short half life. For example, the half-life of bismuth-213 is 46 minutes. After four hours, Bismuth-213 radioactivity falls to negligible levels.

In one aspect of this embodiment, monoclonal antibodies to HIV's gp120 and gp41 envelope proteins are tagged with bismuth 213 and rhenium 188 respectively, with bismuth 213 being particularly preferred. In another aspect of this embodiment, the antibody is an antibody to the gp41 protein designed to bind to the 246-D region (a conserved sequence present across a wide range of genetically diverse HIV strains). A gp41 antibody can be preferred, because its corresponding gp41 antigen is reliably expressed on the surface of cells infected with HIV. Further, unlike other HIV-related glycoproteins, gp41 antigen usually is not shed into the bloodstream, which would lead many of radioactive-labeled antibodies to miss their target.

In combination, the ARVs, the viral replication agents (VRAs), and the RIT will eradicate the HIV virus from a patient's body if used according to the following protocol. The general theory is to allow for a lead-in period with ARVs alone, then to cycle the VRAs and the RIT according to a pre-designated timeline that is based on viral load measurements. By killing off more cells per round of therapy than are being infected, eventually the virus can be purged from the body.

In addition to radiolabeled antibodies, targeted cytotoxic agents include antibodies chemically labeled with maytansines, such as DM1 (mertansine) and DM4, auristatins, such as MMAE and MMAF, calicheamicin, duocarmycin, doxorubicin, and type 1 and 2 ribosome inactivating proteins (RIPs), such as trichosanthin, luffin, ricin, agglutinin, abrin.

The eradication protocol is as follows:

From time point zero, from when a patient is identified as a candidate for eradication therapy, the patient would be put on the daily ARV regimen alone for 6 months. Candidates for ARV therapy would be those recently infected that are still naïve to ARVs, as well any patient for whom resistance testing indicates that the above described ARV regimen can be assembled such that all agents are fully active. All candidates must also have an R5 tropic virus as indicated by a clinically validated tropism assay so that the CCR5 antagonist component of the ARV regimen is fully effective.

At 6 months, provided that the patient has an undetectable viral load (<48 copies/mL), the patient would begin receive a dose of both the VRAs (and maybe also HDACis) and the targeted cytotoxic agent, such as RIT therapeutics, as adjuvant therapy.

The provider would then monitor the patient's viral load every 2 weeks until the patient's viral load returns to undetectable (<48 copies/mL). At that point, the doctor would administer another dose of the adjuvants.

The cycle of administration of adjuvants and viral load monitoring should continue for a minimum of 9 rounds.

At the end of 9 rounds of therapy, patients will be checked by a single-copy VL assay to determine viral persistence. If virus is detected, three more cycles of therapy are recommended. If virus is not detected, then patients will be taken off all drugs, including the ARVs, and monitored with viral load tests every 2 weeks.

Patients with persistent absence of VL detection after 3 months may then be monitored less frequently for up to a year for re-emergence of virus. If no re-emergence is detected, then the virus has likely been purged from the patient's system.

If re-emergence is detected at any point, then the therapy may not work for this patient. Not all patients will be successful with this adjuvant approach.

The VRAs specifically target latent CD4 cells in the lymph, lymph nodes, and secondary lymphoid tissues, including the spleen and GALT (gut-associated lymphoid tissue). Additionally, the 6-month lead-in therapy should ameliorate viral reduction in the CNS, the seminal compartment, and the macrophage and dendritic cell populations.

Additional information on targeted cytotoxic agents is provided below:

Cytotoxic chemicals that can be chemically linked to targeted antibodies include, but are not limited to, maytansines, such as DM1 (mertansine) and DM4, auristatins, such as MMAE and MMAF, calicheamicin, duocarmycin, doxorubicin, and type 1 and 2 ribosome inactivating proteins (RIPs), such as trichosanthin, luffin, ricin, agglutinin, abrin, and radioactive elements such as bismuth 213.

Additional Information on Radioimmunotherapy is Provided Below:

Radioimmunotherapy (RIT) is a therapeutic modality which uses antibody-antigen interaction and antibodies radiolabeled with therapeutic radioisotopes.

Although radiolabeled antibodies to HIV envelope proteins are not effective at killing HIV particles, such therapy is effective at killing cells that harbor HIV. This, in combination with the other aspects of the methods described herein, allows for elimination of persistent reservoirs of HIV-infected cells, which serve as sites of viral synthesis and latency.

In one embodiment, the present invention provides a method for treating a subject infected with HIV which comprises administering to the subject an amount of first compound or series of compounds that activate quiescent cells containing provirus, while also providing HAART to limit new infection by any virus which is expressed by the cells, and a second compound which is a radiolabeled antibody effective to kill HIV infected cells. In one aspect of this embodiment, the antibody is specific for a HIV envelope glycoprotein, and specifically binds to cells that are infected with HIV virus and that express the HIV envelope glycoprotein to which the antibody specifically binds.

In one aspect of this embodiment, the patients are administered a radiolabeled antibody or agent effective to kill HIV infected cells, where the antibody or agent is specific for a HIV envelope glycoprotein. Radioimmunotherapy for HIV is disclosed, for example, in EP1868639A4, the contents of which are hereby incorporated by reference in their entirety for all purposes.

The radioimmunotherapy can be administered in dosage form, comprising a radiolabeled antibody and/or a radiolabeled agent, such as a peptide or an aptamer, and a pharmaceutically acceptable carrier, wherein the antibody and the agent are specific for a HIV envelope glycoprotein and the dosage is appropriate to kill cells infected with HIV in a subject.

In one aspect of this embodiment, the antibody is specific for a HIV envelope antigen (protein or polysaccharide) and specifically binds to cells that are infected with HIV virus and that express the HIV envelope antigen (protein or polysaccharide) to which the antibody specifically binds.

As used herein, the term “antibody” encompasses whole antibodies and fragments of whole antibodies wherein the fragments specifically bind to a HIV envelope protein. Antibody fragments include, but are not limited to, F(ab′)2 and Fab′ fragments and single chain antibodies. F(ab′)2 is an antigen binding fragment of an antibody molecule with deleted crystallizable fragment (Fc) region and preserved binding region. Fab′ is ½ of the F(ab′)2 molecule possessing only ½ of the binding region. The term antibody is further meant to encompass polyclonal antibodies and monoclonal antibodies. The antibody can be, e.g., a neutralizing antibody or a non-neutralizing antibody. Preferably, the antibody is a non-neutralizing antibody, since neutralizing antibodies often bind to highly variable motifs in viral antigens that are vulnerable to antigenic variation.

The antibody can be, e.g., any of an IgA, IgD, IgE, IgG, or IgM antibody. The IgA antibody can be, e.g., an IgA1 or an IgA2 antibody. The IgG antibody can be, e.g., an IgG1, IgG2, IgG2a, IgG2b, IgG3 or IgG4 antibody. A combination of any of these antibodies subtypes can also be used. One consideration in selecting the type of antibody to be used is the desired serum half-life of the antibody. IgG has a serum half-life of 23 days, IgA 6 days, IgM 5 days, IgD 3 days, and IgE 2 days. Another consideration is the size of the antibody. For example, the size of IgG is smaller than that of IgM allowing for greater penetration of IgG into tissues. IgA, IgG, and IgM are preferred antibodies.

The antibody can be specific for any HIV envelope protein, e.g. glycoprotein gp120, gp41 or gp100. Glycoprotein gp160 is a precursor polypeptide, which when cleaved forms gp120 and gp41. The antibody can target protein or polysaccharide epitopes. Combinations of different antibodies can be used, where each different antibody binds to a different epitope. The HIV can be any subtype of HIV, e.g. HIV type 1 or HIV type 2. HIV type 1 induces AIDS. HIV type 2 also leads to immune suppression; however, HIV-2 is not as virulent as HIV-I. Numerous antibodies that bind to a HIV envelope protein have been described (e.g., U.S. Pat. No. 5,731,189; Nadas A, Zhong P, Burda S, Zekeng L, Urbanski M, Gorny M K, Zolla-Pazner S, Nyambi P N. Defining human immunodeficiency virus (HIV) type 1 immunotypes with six human monoclonal antibodies. AIDS Res Hum Retroviruses. 20(1):55-65, 2004; Nyambi P N, Mbah H A, Burda S, Williams C, Gorny M K, Nadas A, Zolla-Pazner S. Conserved and exposed epitopes on intact, native, primary human immunodeficiency virus type 1 virions of group M. J Virol. 74(15):7096-7107, 2000a; Pincus et al., 2003, supra; Till M A, Zolla-Pazner S, Gomy M K, Patton J S, Uhr J W, Vitetta E S. Human immunodeficiency virus-infected T cells and monocytes are killed by monoclonal human anti-gp41 antibodies coupled to ricin A chain. Proc Natl Acad Sci USA. 86(6):1987-91, 1989; Xu J Y, Gorny M K, Palker T, Karwowska S, Zolla-Pazner S. Epitope mapping of two immunodominant domains of gp41, the transmembrane protein of human immunodeficiency virus type 1, using ten human monoclonal antibodies. J Virol. 65(9): 4832-8, 1991; Zolla-Pazner S. Identifying epitopes of HIV-I that induce protective antibodies. Nature Reviews Immunology 4: 199-210, 2004; and U.S. Pat. Nos. 5,731,189, 6,241,986 and 6,395,275).

The antibody is preferably a human antibody. However, the antibody can be a non-human antibody such as a goat antibody or a mouse antibody. It is believed that certain non-human antibodies, including humanized antibodies, can be used in subjects infected with HIV, due to the immune system suppression that occurs in HIV infected subjects.

The molecule carrying the radioactive isotope need not be immunoglobulin since all that is required is a molecule with specificity for binding to a viral antigen expressed on a virally infected cell. Although such molecules are usually proteins, there is no exclusionary requirement for this type of compound and it is conceivable that polysaccharides, lipids, and even small synthetic molecules can be designed to deliver targeted cytotoxic radiation.

Antibodies can be “humanized” using standard recombinant DNA techniques. By transferring the mouse antibody binding site coding region into a human antibody gene, a “human antibody” can be engineered which retains the specificity and biological effects of the original mouse antibody but has the potential to be nonimmunogenic in humans. Additionally, antibody effector functions can be improved through manipulation of the antibody constant region genes. Humanized monoclonal antibodies to gp120 have been described (Dezube B J, Doweiko J P, Proper J A, Conway B, Hwang L, Terada M, Leece B A, Ohno T, Mastico R A. Monoclonal antibody hNMO1 in HIV-infected patients: a phase I study. J Clin Virol. 31 Suppl 1:S45-7, 2004; Major J G, Liou R S, Sun L K5 Yu L M, Starnes S M, Fung M S, Chang T W, Chang N T. Construction and characterization of chimeric and humanized forms of a broadly neutralizing monoclonal antibody to HIV-I. Hum Antibodies Hybridomas 5(1-2):9-17, 1994). An anti-gp120 humanized monoclonal antibody has been shown to be well tolerated in human subjects in a phase I study (Dezube et al., 2004, supra).

The invention can also be practiced using a radiolabeled agent effective to kill HIV infected cells, wherein the agent is specific for a HIV envelope antigen and wherein the radiolabeled agent specifically binds to cells that are infected with HIV virus and that express the HIV envelope antigen to which the agent specifically binds. The chemical composition of the antigen can be, e.g., protein or polysaccharide. Examples of agents that bind to HIV envelope antigens include peptides and aptamers. The agent can be, e.g., a neutralizing agent or a non-neutralizing agent. Preferably, the agent is a non-neutralizing agent.

Examples of HIV envelope glycoprotein-binding peptides include Fuzeon® and retrocyclin-1. Fuzeon® (also known as T-20 or enfuvirtide) is a C-peptide derived from the gp41 C-terminal heptad repeat (CHR) region and is the first member of a new class of anti-HIV drugs known as HIV fusion inhibitors. T-20 may inhibit HIV-I entry by targeting multiple sites in gp41 and gp120 (Liu et al., 2005). Retrocyclin-1 is a theta-defensin peptide which binds to gp120. Neutralizing (Khati M, Schuman M, Ibrahim J, Sattentau Q, Gordon S, James W. Neutralization of infectivity of diverse R5 clinical isolates of human immunodeficiency virus type 1 by gp120-binding 2′F-RNA aptamers. J Virol. 77(23): 12692-8, 2003) and non-neutralizing (Sayer N, Ibrahim J, Turner K, Tahiri-Alaoui A, James W. Structural characterization of a 2′F-RNA aptamer that binds a HIV-I SU glycoprotein, gp120. Biochem Biophys Res Commun. 293(3):924-31, 2002) aptamers that bind to gp120 have been described. A neutralizing antibody or agent is one that reacts with a HIV envelope protein and destroys or inhibits the infectivity and/or virulence of the HIV virus. Methods for generating peptides (Valadon, P., G. Nussbaum, L. F. Boyd, D. H. Margulies, and M. D. Scharff. Peptide libraries define the fine specificity of anti-polysaccharide antibodies to Cryptococcus neoformans. J. Mol Biol. 261:11-22, 1996) and aptamers (U.S. Pat. No. 5,756,291) have been described.

The antibody or agent can also target an antigen that is expressed in HIV-infected cells, but not in non-HIV-infected cells, where the antigen may have viral, mammalian, or combined origin.

Two characteristics are important in the choice of a radioisotope-emission range in the tissue and half-life. Preferably, the antibody or agent is radiolabeled with an alpha emitter or a beta emitter. Alpha emitters have a short emission range in comparison to beta emitters. Examples of alpha emitters include ²¹³-Bismuth (half-life 46 minutes), ²²³-Radium (half-life 11.3 days), ²²⁴-Radium (half-life 3.7 days), ²²⁵-Radium (half-life 14.8 days), 225-Actinium (half-life 10 days), 212-Lead (half-life 10.6 hours), ²¹²-Bismuth (half-life 60 minutes), 211-Astatine (half-life 7.2 hours), and ²⁵⁵-Fermium (half-life 20 hours). A preferred alpha emitter is ²¹³Bi, which emits a high LET α-particle with E=5.9 MeV with a path length in tissue of 50-80 μm. Theoretically a cell can be killed with one or two α-particle hits. ²¹³Bi is the only α-emitter that is currently available in generator form, which allows transportation of this isotope from the source to clinical centers within the United States and abroad.

Examples of beta emitters include ¹⁸⁸-Rhenium (half-life 16.7 hours), ³²-Phosphorous (half-life 14.3 days), ⁴⁷-Scandium (half-life 3.4 days), ⁶⁷-Copper (half-life 62 hours), ⁶⁴-Copper (half-life 13 hours), ⁷⁷-Arsenic (half-life 38.8 hours), ⁸⁹-Strontium (half-life 51 days), ¹⁰⁵-Rhodium (half-life 35 hours), ¹⁰⁹-Palladium (half-life 13 hours), ¹¹¹-Silver (half-life 7.5 days), ¹³¹-Iodine (half-life 8 days), ¹⁷⁷-Lutetium (half-life 6.7 days), ¹⁵³-Samarium (half-life 46.7 hours), ¹⁵⁹-Gadolinium (half-life 18.6 hours), ¹⁸⁶-Rhenium (half-life 3.7 days), ¹⁶⁶-Holmium (half-life 26.8 hours), ¹⁶⁶-Dysprosium (half-life 81.6 hours), ¹⁴⁰-Lantanum (half-life 40.3 hours), ¹⁹⁴-Irridium (half-life 19 hours), ¹⁹⁸-Gold (half-life 2.7 days), ¹⁹⁹-Gold (half-life 3.1 days), ⁹⁰-Yttrium (half-life 2.7 days), ¹⁷⁷-Lutetium (half-life 6.7 days) and ¹³¹-Iodine (half-life 8 days). Preferred beta emitters include ¹³¹-Iodine, ⁹⁰-Yttrium, ¹⁸⁸-Rhenium, ¹⁸⁶-Rhenium, ¹⁷⁷-Lutetium, ¹⁶⁶-Holmium, ⁶⁷-Copper, and ⁶⁴-Copper, with the high-energy β-emitter ¹⁸⁸-Rhenium (Eₘₐₓ=2.12 MeV) being most preferred. ¹⁸⁸-Re has the additional advantage that it emits γ-rays which can be used for imaging studies.

The radioisotope can be attached to the antibody or agent using any known means of attachment used in the art, including interactions such as avidin-biotin interactions, “direct” radiolabeling and radiolabeling through a bifunctional chelating agent (Saha G B Fundamentals of Nuclear Pharmacy, Springer, New York, pp. 139-143, 1997). Preferably, the radioisotope is attached to the antibody or agent before the radioisotope or the antibody or agent is administered to the subject.

Combinations of antibodies and/or agents radiolabeled with different radiolabels can be used, for example, where the radioisotopes are isotopes of a plurality of different elements. In one aspect of this embodiment, at least one radioisotope in the plurality of different radioisotopes is a long range (beta) emitter and at least one radioisotope is a short range (alpha) emitter. Preferably, the beta emitter is ¹⁸⁸-Rhenium. Preferably, the alpha emitter is ²¹³-Bismuth.

It is known from radioimmunotherapy studies of tumors that whole antibodies usually require from 1 to 3 days time in circulation to achieve maximum targeting. While slow targeting may not impose a problem for radioisotopes with relatively long half-lives such as ¹⁸⁸-Re (t_{1/2}2=16.7 hours), faster delivery vehicles may be preferred for short-lived radioisotopes such as ²¹³-Bi 04/2=46 min). The smaller F(ab′)2 and Fab′ fragments or domain-deleted antibodies provide much faster targeting which matches the half-lives of short-lived radionuclides (Milenic, 2000; Buchsbaum, 2000). A ‘domain-deleted’ antibody is an antibody from which a particular domain, e.g. CH2, has been deleted and replaced with a peptide linker for the purpose of optimizing its therapeutic potential (Milenic, D. E. Radioimmunotherapy. designer molecules to potentiate effective therapy. Semin. Radiat. Oncol. 10: 139-155, 2000).

In order to calculate the dose of the radioisotope which can significantly decrease or eliminate infection burden without radiotoxicity to vital organs, a diagnostic scan of the patient with the antibody or agent radiolabeled with diagnostic radioisotope or with low activity therapeutic radioisotope can be performed prior to therapy, as is customary in nuclear medicine. The dosimetry calculations can be performed using the data from the diagnostic scan.

Fractionated doses of radiolabeled antibodies can be more effective than single doses and can be less radiotoxic to normal organs. Depending on the status of a patient and the effectiveness of the first treatment with RIT the treatment can consist of one dose or several subsequent fractionated doses.

The dose of the radioisotope for humans will typically be between about 1-500 mCi.

The radiolabeled antibody or agent can be delivered to the subject by a variety of means. Preferably, the radiolabeled antibody or agent is administered parenterally. The radiolabeled antibody or agent can be injected, for example, into the bloodstream, into a muscle or into an organ such as the spleen.

The HIV-infected cell that is targeted and killed by the radiolabeled antibody or agent can be any of, e.g., but not limited to, a lymphocyte, such as a T lymphocyte or a CD4+ T lymphocyte, a monocyte, a macrophage, an astrocyte and/or a microglial cell.

The present invention will be better understood with reference to the following non-limiting example.

### Example 1

### Mathematical Model of Combined IAT/HAART

While antiretroviral drugs can drive HIV-1 to undetectably low levels in the blood, eradication is hindered by the persistence of long-lived, latently infected memory CD4 T cells. Immune activation therapy aims to eliminate this latent reservoir by reactivating these memory cells, exposing them to removal by the immune system and the cytotoxic effects of active infection.

A mathematical model is described that investigates the use of immune activation strategies while limiting virus and latent class rebound. This model considers infection of two memory classes, central and transitional CD4 T cells, and the role that general immune activation therapy has on their elimination. Further, the model incorporates ways to control viral rebound by blocking activated cell proliferation through anti-proliferation therapy. Using the model, control of latent infection is described, which subsequently can lead to the long-term control of HIV-1 infection.

### INTRODUCTION

Administration of highly active antiretroviral therapy (HAART) in HIV-1 infected patients has led to successful reduction of HIV-1 RNA levels to below the limits of detection (<50 viral copies per mL) (Dornadula et al. 1999; Di Mascio et al. 2003; Zhang et al. 1999). Loss of active virus replication ensures protection against emergence of drug resistant viral strains and allows for immune reconstitution (Kaufmann et al. 2000; Lok et al. 2010). However, many patients on HAART experience episodes of low-level viremia (blips) followed by a return to undetectable levels without a change in therapy (Di Mascio et al. 2005; Nettles et al. 2005). Such events suggest that virus is not completely cleared from infected individuals and persists at low-levels for many years (Dornadula et al. 1999; Palmer et al. 2008). The proposed mechanisms of viral persistence are HAART's inability to completely suppress viral replication (Blankson et al. 2002; Gunthard et al. 2001) and the existence of long-lived HIV-1 reservoirs in resting memory CD4 T cells (Chun et al. 1997b; Finzi et al. 1997). This paper will focus on the role of the latent reservoir in viral persistence and on the effects of immune therapies in controlling latent populations.

The establishment of the latent CD4 T cell reservoir occurs during primary infection, before seroconversion and the start of HAART treatment (Chun et al. 1998). HIV-1 infects activated CD4 T cells and, following reverse transcription of RNA into DNA, enters the nucleus and integrates into the host genome, initiating rounds of viral replication (Freed and Martin 2007).

While most activated CD4 T cells with integrated HIV-1 are short-lived due to virus cytopathicity and host immune responses, a small fraction revert to a resting state, become memory cells, and do not produce virus (Chun et al. 1997a). Integrated HIV-1 DNA persists in these long-lived latent reservoirs with an estimated half-life of up to 44 months (Finzi et al. 1999; Siliciano et al. 2003). These latently-infected cells remain invisible to the immune response when in a resting state, and serve as a source of new infectious particles when activated. Occasional antigen-specific activation of these memory cells can explain the viral blips observed in patients (Di Mascio et al. 2005; Jones and Perelson 2007). Because such cells cannot be distinguished from uninfected cells by immune responses and occasional activation leads to viral rebound, complete removal of the latent reservoir is required before HAART therapy can be interrupted (Harrigan et al. 1999).

Recent studies have identified the existence of two distinct HIV-1 reservoirs: central memory CD4 T cells (TCM) and transitional memory CD4 T cells (TTM). TCM are long-lived cells maintained through T cell survival signals and low-level antigen-induced proliferation. In response to T cell receptor triggering and homeostatic signals, TCM can differentiate into effector memory T cells (TEM) (Sallusto et al. 2004). TTM cells present an intermediate phenotype with a slightly shorter half-life than TCM, and undergo homeostatic proliferation in the presence of large amounts of IL-7 (Chomont et al. 2009, 2011). The relative proportion of these two populations is highly dependent on the absolute CD4 T cell count, with TCM being the predominant form of latently infected cells in patients with high CD4 T cell count. Conversely, TTM undergoing homeostatic proliferation predominate in patients with low CD4 T cell counts. Achieving viral eradication requires therapeutic strategies targeting both of these populations through activation of memory cells and blockage of the self-renewal and persistence of proliferating memory T cells (Chomont et al. 2009, 2011).

When memory cells infected with HIV-1 encounter an antigen for which they are specific, they become activated, proliferate, and begin producing HIV-1 virions. In patients on HAART treatment, these cells are eliminated by the immune system or by the cytolytic effects of HIV-1, and the new virions produced have a minimal effect on the course of the disease or treatment. Immune activation therapy proposes to artificially induce the activation of many memory T cells in the absence of their specific antigens, thus reducing the size of the latent pool much more quickly than the random natural process (Burnett et al. 2010; Geeraert et al. 2008; Lehrman et al. 2005; Siliciano et al. 2007).

A combination of therapeutic reactivation of latent infections with antiretroviral treatments may accelerate the depletion of latent reservoirs (Geeraert et al. 2008). However, such latency reactivation strategies have yielded variable results in recent clinical trials. Activation can induce viral replication (Chun et al. 1999; Davey et al. 1997), increase the number of susceptible uninfected target cells beyond the threshold that can be contained by antiretroviral therapy, and replenish the latent reservoir (Burnett et al. 2010; Lehrman et al. 2005; Siliciano et al. 2007).

New studies are investigating whether some agents can induce HIV-1 expression while simultaneously limiting any activation of CD4 T cells (Margolis 2010; Mellberg et al. 2011; Kovochich et al. 2011; Wolschendorf et al. 2010). As latent populations are difficult to detect clinically in the absence of activation, the mathematical models described herein are an alternative approach to studying latent pool dynamics.

The population dynamics of target and infected cell types in an HIV-1-infected patient are modeled by extending classical HIV-1 models (Ho et al. 1995; Wei et al. 1995; Perelson et al. 1997) to include latently infected central and transitional memory CD4 T cells. Using such models, whether therapeutic strategies aimed at activating the latent pool while limiting proliferation have the potential to reduce the viral reservoir can be investigated. Models for mechanisms leading to viral latency and persistence in central and transitional memory cells can be developed, and used to investigate how cell activation and inhibition of proliferation affect the dynamics of the overall latent populations.

Previous models have investigated mechanisms causing viral load persistence (Jones and Perelson 2007; Kim and Perelson 2006; Rong and Perelson 2009a; Yates et al. 2007), the dynamics of the latent reservoir and the presence of viral blips (Di Mascio et al. 2005; Rong and Perelson 2009b, 2009c; Smith and Aggarwala 2009) and the role of immune activation therapy in reducing the latent reservoir (Sedaghat et al. 2008; Fraser et al. 2000). In this paper, such models are expanded to account for latent infection of central and transitional memory cells.

In Sect. 2, a mathematical model of viral latency is developed that addresses the relationship between abundance of central and transitional cells and the total CD4 T cells count at the start of HAART and accounts for immune activation therapy. The models are evaluated and analytical results presented. In Sect. 3, numerical results are presented and biological predictions made based on the model's assumptions. In particular, the potential benefits of immune activation therapy are addressed, and ways to limit latent cell division due to activation and homeostatic expansion are discussed.

### 2 A Mathematical Model of Latent Infection

The model considers a target cell population T, consisting of combined uninfected resting and activated CD4 T cells. It is assumed that at any time, a fraction p of uninfected CD4 T cells are activated and, therefore, susceptible to infection. Their growth is modeled by a Michaelis-Menten type term sT/(bT+T+I) to account for more frequent division when the total CD4 T cell population T+I is small and a saturating growth rate when the total CD4 T cell population T+I reaches the maximum value observed in the periphery. s is the maximum rate at which uninfected CD4 T cells are produced from thymus or through homeostatic expansion, and bT is the total T cell concentration corresponding to half-maximal T cell production. Uninfected cells die at a rate d=pdA+(1−p)dR per day, where dA and dR are the rates of activated and resting CD4 T cells, respectively, and become infected at rate β=β^{˜}p where β^{˜} is the infection rate for activated CD4 T cells.

Upon infection, a fraction 1−ƒ become productively infected cells, I, which die at rate δ. The remaining fraction ƒ revert to a resting state, acquire memory phenotype, become latently infected central memory cells LC and die at rate dC. In response to homeostatic signals, a fraction θ of central memory cells become transitional memory LT, which proliferate at rate α and die at rate dT.

The movement from central memory to transitional memory and the proliferation are dependent on the density of CD4 T cell population, with bL being the T cell concentration resulting in half-maximal expansion. The total T cell population is modeled by T+I, as in the equation for T, as the memory population is small relative to the total T cell pool (Chun et al. 1997a), so that the total CD4 population is approximately T+I. Random antigen-dependent activation events of both central and transitional memory cells occur at rate a. Once activated, latent cells expand by a factor of ρ, become productively infected cells, and start producing new viruses. An infected cell produces N HIV-1 RNAs during its lifespan, and the virus is cleared at rate c.

The dynamics of latent infection are investigated during HAART therapy that inhibits HIV-1 infection with efficacy ε. Anti-proliferation drugs reduce the expansion of activated and proliferating memory cells by a factor η. These dynamics are described by the following system of differential equations:

$$\begin{matrix}\frac{d T}{d t} = \frac{sT}{{b}_{T} + T + I} - d T - ( 1 - ɛ ) β TV & ( 1 a ) \\ \frac{d I}{d t} = ( 1 - f ) ( 1 - ɛ ) β TV - δ I + a ρ ( 1 - η ) ( {L}_{C} + {L}_{T} ) & ( 1 b ) \\ \frac{d V}{d t} = N δ I - cV & ( 1 c ) \\ \frac{d {L}_{C}}{d t} = ( 1 - ɛ ) f β TV - \frac{θ {L}_{C}}{{b}_{L} + T + I} - ( {d}_{C} + a ) {L}_{C} & ( 1 d ) \\ \frac{d {L}_{T}}{d t} = \frac{θ {L}_{C} + ( 1 - η ) α {L}_{T}}{{b}_{L} + T + I} - ( {d}_{T} + a ) {L}_{T} . & ( 1 e )\end{matrix}$$

It is assumed that the target population initially consists of normal CD4 T cell levels T (0)=TO. In the absence of infection I (0)=LC(0)=LT (0)=0, and the initial viral inoculum is V (0)=V0. The asymptotic behavior of the system is determined in the absence of therapy, and the asymptotic values are used as new initial conditions for the system in the presence of drug therapy.

2.1 Analysis of the Base System in the Absence of Activation 2.1.1 Existence of Steady States

The model begin by studying the system in the absence of random activation events (a=0). For convenience, the model defines K=s/d−bT. If s/d>bT, K denotes the carrying capacity of the uninfected T cell population in the absence of virus. Under the assumption that a=0, there are three potential steady states: E0=(0, 0, 0, 0, 0), the viral clearance steady state E1=(K, 0, 0, 0, 0), which exists only if K>0, and a chronic infection steady state E2=( T̅, Ī, V̅, L̅_{C}, L̅_{T}) with positive components. For the chronic state E2,

$$\overline{T} = \frac{c}{( 1 - f ) ( 1 - ɛ ) β N} .$$

Ī the solves the quadratic equation AĪ²+BĪ+C=0, with

$$A = \frac{δ}{1 - f}$$

$$B = s \overline{T} + \frac{δ}{1 - f} ( {b}_{T} + \overline{T} ) , and$$

$$C = - d \overline{T} ( K - \overline{T} ) .$$

This quadratic equation has a unique positive solution if and only if C<0, which can be written as

$${R}_{0} ≡ \frac{K}{\overline{T}} = \frac{( 1 - f ) ( 1 - ɛ ) β NK}{c} > 1.$$

When this condition is satisfied, we have

$$\overline{I} = \frac{1}{2} \{ - ( \frac{c d}{( 1 - ɛ ) β N δ} + {b}_{T} + \overline{T} ) + \sqrt{{( \frac{c d}{( 1 - ɛ ) β N δ} + {b}_{T} + \overline{T} )}^{2} + 4 \frac{c d}{( 1 - ɛ ) β N δ} ( K - \overline{T} )} \} .$$

The remaining steady state values are then given by

$$\overline{V} = \frac{N δ \overline{I}}{c} , \\ {\overline{L}}_{C} = \frac{( 1 - ɛ ) f β \overline{T} \overline{V}}{\frac{θ}{{b}_{L} + \overline{T} + \overline{I}} + {d}_{C}} , and$$

$${\overline{L}}_{T} = \frac{θ {\overline{L}}_{C}}{{d}_{T} ( {b}_{L} + \overline{T} + \overline{I} ) - α} .$$

The last value exists and is positive if and only if: α<d_{T}(b_{L}+ T̅+Ī).

### 2.1.2 Linear Stability Analysis

In the absence of activation, the Jacobian of system (1a)-(1e) takes the block diagonal form

$$𝒥 = [ \begin{matrix}{𝒥}_{1} & 0 \\ {𝒥}_{3} & {𝒥}_{2}\end{matrix} ] , \\ with$$

$${𝒥}_{1} = [ \begin{matrix}\frac{s ( {b}_{T} + 1 )}{{( {b}_{T} + T + I )}^{2}} - d - ( 1 - ɛ ) β V & - \frac{sT}{{( {b}_{T} + T + I )}^{2}} & - ( 1 - ɛ ) β T \\ ( 1 - f ) ( 1 - ɛ ) β V & - δ & ( 1 - f ) ( 1 - ɛ ) β T \\ 0 & N δ & - c\end{matrix} ]$$

$$and$$

$${𝒥}_{2} = [ \begin{matrix}- \frac{θ}{{b}_{L} + T + I} - {d}_{C} & 0 \\ \frac{θ}{{b}_{L} + T + I} & \frac{α}{{b}_{L} + T + I} - {d}_{T}\end{matrix} ] .$$

Proposition 1

When the viral clearance state E1 exists, i.e., K>0, the zero steady state E0 is unstable. Furthermore, if R0<1 and

$$\frac{α}{{b}_{L} + K} < {d}_{T} . \qquad ( 3 )$$

then E1 is locally asymptotically stable.

Analysis

When T=I=V=0, dK/bT is an eigenvalue of the Jacobian and is positive if and only if E1 exists. It follows that E0 is unstable when E1 exists. To establish the stability of E1, we must show that all of the eigenvalues of the Jacobian J have negative real part when it is evaluated at E1. The eigenvalues of J2 are clearly negative under the conditions of Proposition 1. At E1, we also have

$${𝒥}_{1} = [ \begin{matrix}- \frac{{d}^{2}}{s} K & - \frac{{d}^{2}}{s} K & - ( 1 - ɛ ) β K \\ 0 & - δ & ( 1 - f ) ( 1 - ɛ ) β K \\ 0 & N δ & - c\end{matrix} ] .$$

The first entry of this matrix is one of its eigenvalues, and is negative. The remaining two eigenvalues are the solutions of

λ²−(δ+c)λ+δc−(1−ƒ)(1−ε)βNδK=0

which have negative real part if and only if the constant term is positive, i.e., of R0<1. The condition (3) has a straightforward biological interpretation: homeostatic proliferation must not continue in an unbounded manner when the T cell population is at its maximum.

Proposition 2

If the chronic steady state E2 exists, then it is locally asymptotically stable.

Proof: As before, the eigenvalues of [symbol] are negative, as the existence condition for E2 is exactly the condition for the diagonal entries of [symbol] to be negative. The focus is on the submatrix [symbol] ₁, which can be rewritten in the form:

$${𝒥}_{1} = [ \begin{matrix}- \frac{s \overline{T}}{{( {b}_{T} + \overline{T} + \overline{I} )}^{2}} & - \frac{s \overline{T}}{{( {b}_{T} + \overline{T} + \overline{I} )}^{2}} & - ( 1 - ɛ ) β \overline{T} \\ \frac{δ \overline{I}}{\overline{T}} & - δ & \frac{c}{N} \\ 0 & N δ & - c\end{matrix} ]$$

Although algebraically tedious, it is straightforward to show that the coefficients of the characteristic equation for [symbol] ₁ satisfy the Routh-Hurwitz criterion. It follows that the steady state E2 is locally asymptotically stable.

2.2 Analysis of the Base System with Activation

When activation rate, a, is nonzero we increase the fraction of activated, uninfected CD4 T cells to p′. This will lead to an increase in the infectivity rate β_=p′/p β. Moreover, the overall death rate of uninfected CD4 T cells becomes d′=p′dA+(1−p′)dR. The analysis of the system is complicated. The model concentrates on determining conditions under which the clearance steady state remains locally asymptotically stable and investigate the effects of activation on latent pool elimination.

### 2.2.1 Linear Stability Analysis

The location of the clearance steady state E1=(K, 0, 0, 0, 0) is unaffected by the value of a, but the Jacobian matrix governing its stability is altered from (2) to

$${𝒥}_{a} = [ \begin{matrix}- \frac{{d}^{2}}{s} K & - \frac{{d}^{2}}{s} K & - ( 1 - ɛ ) β K & 0 & 0 \\ 0 & - δ & ( 1 - f ) ( 1 - ɛ ) β K & a ρ ( 1 - η ) & a ρ ( 1 - η ) \\ 0 & N δ & - c & 0 & 0 \\ 0 & 0 & - ( 1 - ɛ ) f β K & - \frac{θ}{{b}_{L} + K} - {d}_{C} - a & 0 \\ 0 & 0 & 0 & \frac{θ}{{b}_{L} + K} & \frac{( 1 - η ) α}{{b}_{L} + K} - {d}_{T - a}\end{matrix} ] . \qquad ( 4 )$$

The characteristic equation of this matrix is

$$( λ + \frac{{d}^{2}}{s} K ) ( {λ}^{4} + W {λ}^{3} + X {λ}^{2} + Y λ + Z ) = 0 \\ where \\ W = {d}_{T} - \frac{α ( 1 - η )}{{b}_{L} + K} + {d}_{C} + 2 a + δ + c + \frac{θ}{{b}_{L} + K} , \\ X = c δ ( 1 - {R}_{0}^{′} ) + ( {d}_{T} - \frac{α ( 1 - η )}{{b}_{L} + K} ) ( \frac{θ}{{b}_{L} + K} + a + c + δ + {d}_{C} ) + {a}^{2} + 2 a ( c + δ ) + ( {d}_{C} + \frac{θ}{{b}_{L} + K} ) ( a + c + δ ) , \\ Y = c δ ( 1 - {R}_{0}^{′} ) ( {d}_{T} - \frac{α ( 1 - η )}{{b}_{L} + K} ) + c δ ( 1 - {R}_{0}^{′} ) ( a + {d}_{C} + \frac{θ}{{b}_{L} + K} ) + ( c + δ ) ( a + {d}_{T} - \frac{α ( 1 - η )}{{b}_{L} + K} ) ( a + {d}_{C} + \frac{θ}{{b}_{L} + K} ) + a c δ ( 1 - {R}_{0}^{′} - {R}_{1} ) , \\ Z = c δ ( 1 - {R}_{0}^{′} ) ( {d}_{T} - \frac{α ( 1 - η )}{{b}_{L} + K} ) ( {d}_{C} + \frac{θ}{{b}_{L} + K} ) + {ad}_{C} c δ ( 1 - {R}_{0}^{′} ) + a c δ ( 1 - {R}_{0}^{′} - {R}_{1} ) ( {d}_{T} - \frac{α ( 1 - η )}{{b}_{L} + K} + a + \frac{θ}{{b}_{L} + K} ) . \qquad ( 5 )$$

The new constants are defined below:

$${R}_{0}^{′} = \frac{{p}^{′}}{p} {R}_{0} = \frac{( 1 - f ) ( 1 - ɛ ) {β}^{′} NK}{c} and$$

$${R}_{1} = \frac{ρ ( 1 - η ) f ( 1 - ɛ ) {β}^{′} NK}{c} .$$

This R1 has a form very similar to the basic reproductive numbers R0 and R′0. It represents the number of new cells entering the latent class as a result of the reactivation of a single latently infected cell. Such a cell produces a progeny of ρ(1−η) actively infected cells, which in turn produce new virus.

When these viruses infect naive T cells, a fraction ƒ reenter the latent class. R′0 gives the average number of new infected cells resulting from a single infected cell in an uninfected host, accounting for the increase availability of activated T cells in an immune activation scenario. Similarly, R1 gives the average number of new latent cells resulting from a single latently infected cell as a result of the process of reactivation and infection. If the sum of these is sufficiently greater than 1, numerical results show that the infection can maintain itself through a combination of activation from the latent reservoirs and active infection.

Proposition 3 shows that if R′0+R1<1, the infection cannot maintain itself.

Proposition 3

If R′0+R1<1 and α/(bL+K)<dT, then the clearance state E1=(K, 0, 0, 0, 0) is locally asymptotically stable.

To establish this result, it was determined that the Routh-Hurwitz criteria apply to the characteristic polynomial given in Eq. (5). Under the conditions of Proposition 3, all of the coefficients of the characteristic polynomial are positive. It can also be shown that the remaining conditions (WX>Y and WXY>Y²+W²Z) are satisfied. The computations are tedious and uninformative, and are therefore omitted. Note that R′0+R1<1 is a sufficient condition for the stability of the clearance state under activation.

### 3 Results

3.1 Parameter Values

In the absence of HIV-1 infection, there are s/bT −d=10⁶ uninfected CD4 T cells per mL (Sachsenberg et al. 1998). The model assumes that uninfected CD4 T cells will reach their half-maximal expansion rate when the total CD4 T cell population is bT=3×105 cells per mL. Resting CD4 T cells are long-lived, dR=0.01 per day (Stafford et al. 2000) and activated CD4 T cells die at a faster rate, dA=0.1 per day. If we assume that a fraction p=7% of cells are activated, as is the case during active HIV-1 infection (Hunt et al. 2008), then the overall death rate is d=pdA+(1−p)dR=0.0163 per day and s=2.12×10⁴ cells per ml per day. Initial inoculum of 0.33 virus per mL leads to infection of CD4 T cells at a rate of β=4.2×10-8 (Stafford et al. 2000).

Of the cells that become infected, the vast majority, 1−ƒ, become activated CD4 T cells, while the remainder, ƒ, become latently-infected central memory CD4 T cells. As the establishment of a latent infection in a cell is a rare event (Chun et al. 1997b, 1997a), we restrict the fraction of cells that become latently infected to ƒ=1.5×10⁻⁶. Actively infected cells are killed by viral cytotoxicity and immune cells at a rate of δ=0.39 per day (Markowitz et al. 2003). An average of N=2000 virions bud off an infected cells during its lifespan (Hockett et al. 1999) and the virus is cleared at a rate of c=23 per day (Ramratnam et al. 1999). When the total CD4 T cell concentration is reduced, latent central memory T cells acquire a different phenotype and become transitional memory cells at a maximal rate of θ=100 cells per mL per day. Transitional memory cells undergo non-specific homeostatic proliferation at a maximal rate of α=2700 cells per mL per day.

Both recruitment and homeostatic proliferation of transitional cells is dependent of the total CD4 T cell density, with a half-maximal expansion rate achieved when the total CD4 T cell population is bL=3×10⁵ cells per mL. Latently infected cells are long-lived. This is modeled by considering average half-lives for central and transitional memory cells to be 44 and 6 months, corresponding to the two extreme estimates found in the literature (Finzi et al. 1999; Siliciano et al. 2003; Chun et al. 2007; Ramratnam et al. 1999; Zhang et al. 1999). Using these values, we calculate the average death rates to be dC=ln(2)/(44·30)=5×10⁻⁴ per day and dT=ln(2)/(44·30)=4×10⁻³ day-1>dC, respectively.

Current HAART drug combinations are very effective (Shen and Siliciano 2008), and are modeled by a 99% reduction in the rate of infection (ε=0.99). Latently infected cells can be activated at rates ranging between a=10-4 and a=10⁻² per day following an encounter with a specific antigen. As this model does not consider clonal specificity, the activation rate takes into account the fraction of cells which are stimulated. For example, a=10⁻² may represent a strong stimulus for a small number of cells. The activated cells move into the actively infected CD4 T cell population after they expand ρ=100 times.

During immune activation therapy, when non-specific activation affects all latently infected memory CD4 T cells, the activation rate is a=10⁻¹ per day. To account for the nonspecific activation of resting T cells and the subsequent increase in susceptibility to HIV-1 infection, the fraction of the cells that are activated is increased to p′ and the overall death rate of uninfected T cells to d′=p_dA+(1−p′)dR to account for shorter life span of activated cells.

We choose p_ such that the new basic reproductive number R′0=p′p R0<1 following activation, given that R0<1. We assume that immune activation therapy is accompanied by antiproliferation therapy which blocks cell proliferation with an efficacy of 99%, or η=0.99.

3.2 Latent Infection

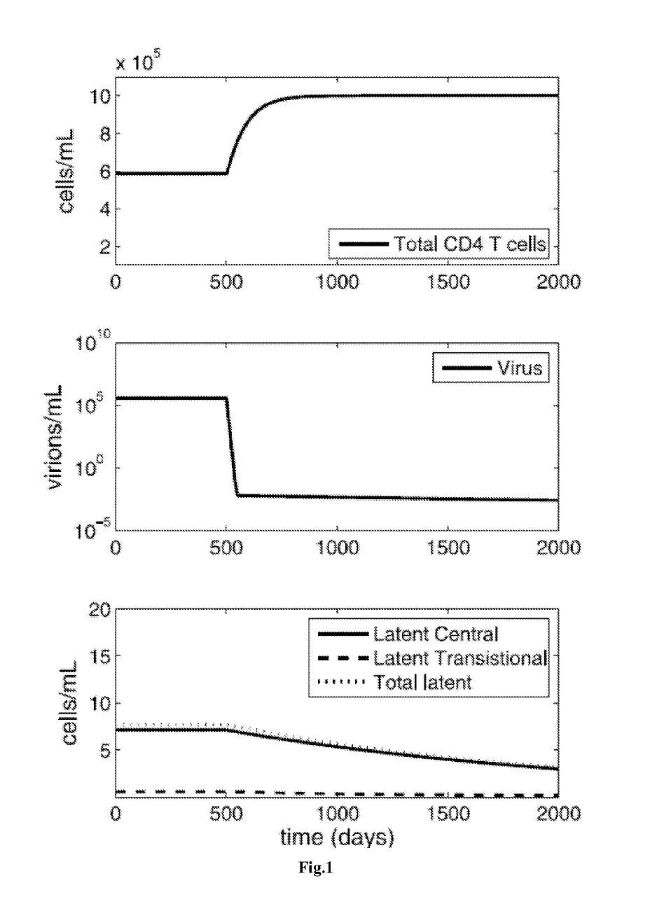

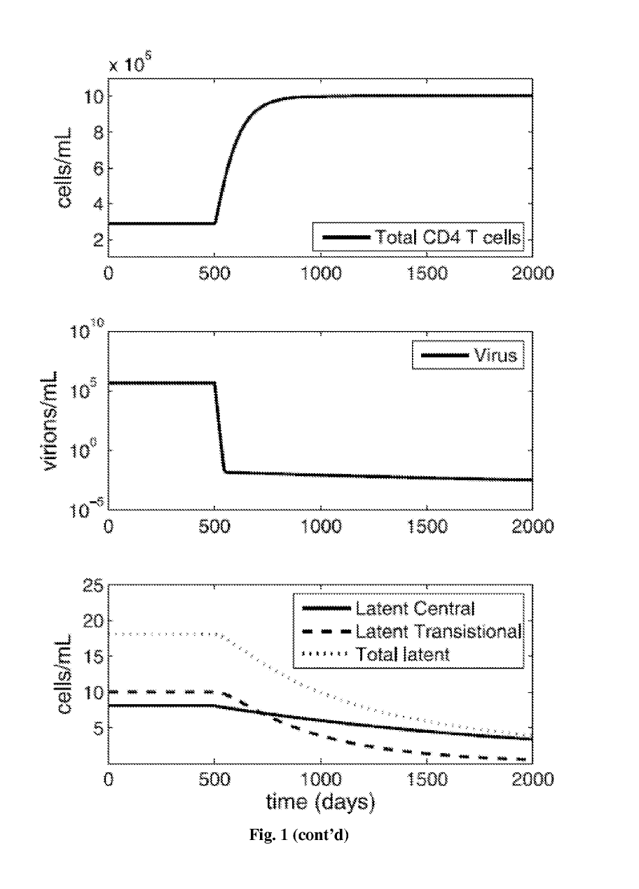

The mathematical model of latent HIV-1 infection described herein considers two classes of latent cells: the central and transitional memory CD4 T cells. In the absence of drug therapy, the level of the total CD4 T cells population is highly dependent on the infectivity rate β (see FIG. 1, top panels, first 500 days). Two values for β are considered, that lead to chronic infections: β=2×10⁻⁸, corresponding to a basic reproductive number of R0=1.8, and β=4.2×10⁻⁸ corresponding to a basic reproductive number of R0=3.6. The total number of T cells in the asymptomatic phase of infection (the steady-state) is 590 cells per μl in the first case (see FIG. 1, top left panel) and 270 cells per μl (close to AIDS values) in the second case (see FIG. 1, top right panel).

The dynamics of latent infection are investigated in both cases with the aim of determining the effect of CD4 T cell density on the dynamics of memory phenotypes. When the total population of T cells is relatively high, the latent class is composed primarily of central memory cells (see solid line in FIG. 1, lower left panel, first 500 days). Conversely, under lymphopenic conditions, the latent class is composed primarily of transitional memory cells (see dashed line in FIG. 1, lower right panel, first 500 days). These results are in agreement with experimental observations (Chomont et al. 2009).

The model assumes that patients start antiretroviral therapy at day 500, which successfully blocks the infection with efficacy ε=0.99. Virus follows a two-phase decay. The fast initial decline corresponds to the loss of virions, and the subsequent slow decline below the limit of detection corresponds to the loss of virus produced as a result of background activation of memory cells (FIG. 1, middle panels), consistent with previous reports (Simon and Ho 2003; Rong and Perelson 2009c, 2009a). At the same time, a rebound in the total number of CD4 T cells to 1000 cells per μl is noticed (FIG. 1, upper panels).

A decrease in drug efficacy alters the rate of viral decay, moving concentrations closer to the limit of detection of 50 viruses per mL, but does not affect the dynamics of latent populations or CD4 T cell recovery. For high pretreatment CD4 counts, HAART leads to a slow exponential decline in the number of latently infected T cells, with central and transitional memory T cells (see FIG. 1, left column) having half-life of 44 and 23 months, respectively. In cases with low pretreatment CD4 counts, the latently infected transitional memory T cell population declines at an increased rate as lymphopenia is relieved. For the chosen parameters, the estimated half-lives are 44 months for the central memory T cells (see solid line in FIG. 1, right column) and 14 months for the transitional memory T cells (see dashed line in FIG. 1, right column). The decreased half-life of transitional memory cells results from the combined effects of loss due to activation and reduced homeostatic proliferation due to the rise in the total T cell population. When a latently infected memory T cell encounters its specific antigen on the surface of antigen presenting cells, it becomes activated.

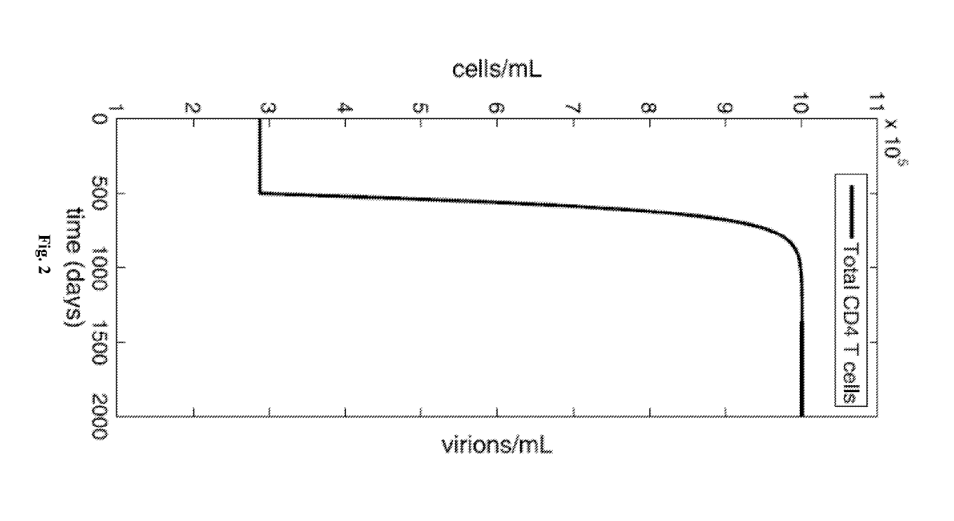

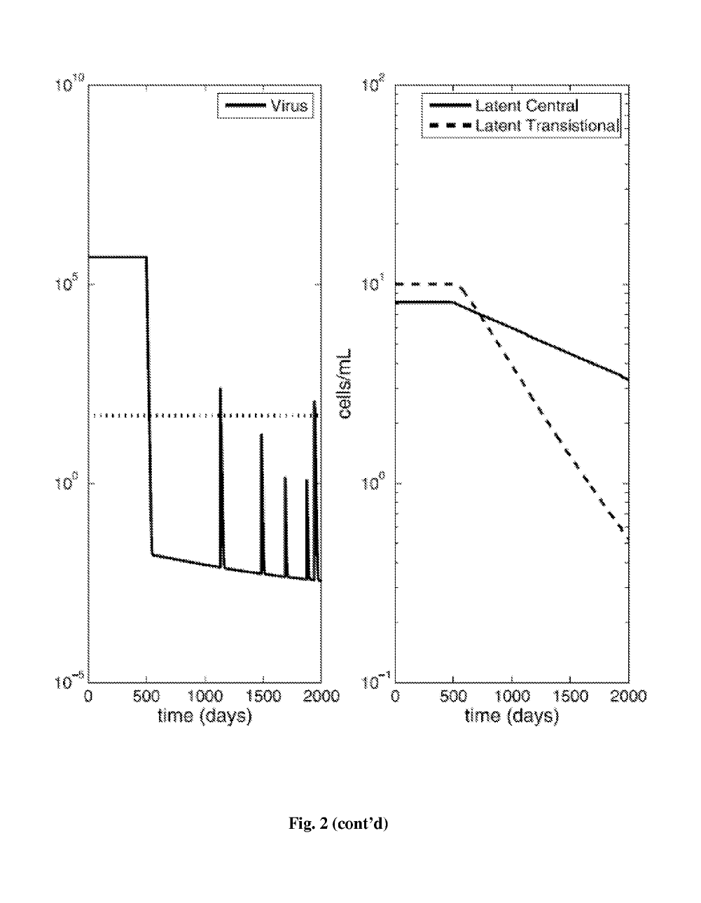

On the population level, this leads to the activation of latently infected cells at rate a. We assume that these are random events. The strength of latent cells activation ranges between 10⁻⁴ and 10⁻², depending on the frequency of cells specific to the given antigen. These activation events lead to generation and proliferation of productively infected cells at rate aρ ranging between 0.01 and 1 per day. As a result, transient spikes (or blips) in HIV-1 levels are observed (see FIG. 2, middle panel). For the small activation rates that are considered, the amplitudes of viral spikes observed in patients by Di Mascio et al. (2005) can be reproduced. These small amplitude viral blips do not impact the dynamics of the latent reservoir or the overall CD4 T cell population (see FIG. 2, left and right panel).

3.3 Immune Therapy

The mathematical framework established in the system of equations (1a)-(1e) is used to explore two immune-based treatments for HIV-1: immune activation and antiproliferation therapies.

3.3.1 Immune Activation Therapy (IAT) Artificially Activates a Large Number of Memory T Cells

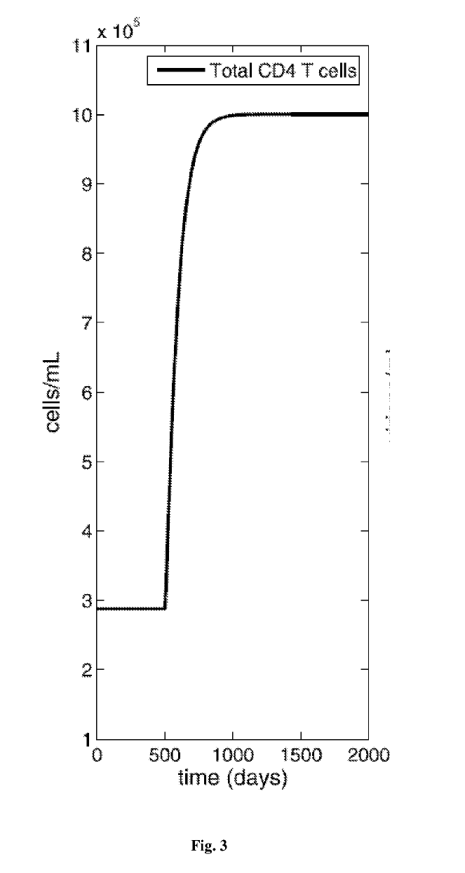

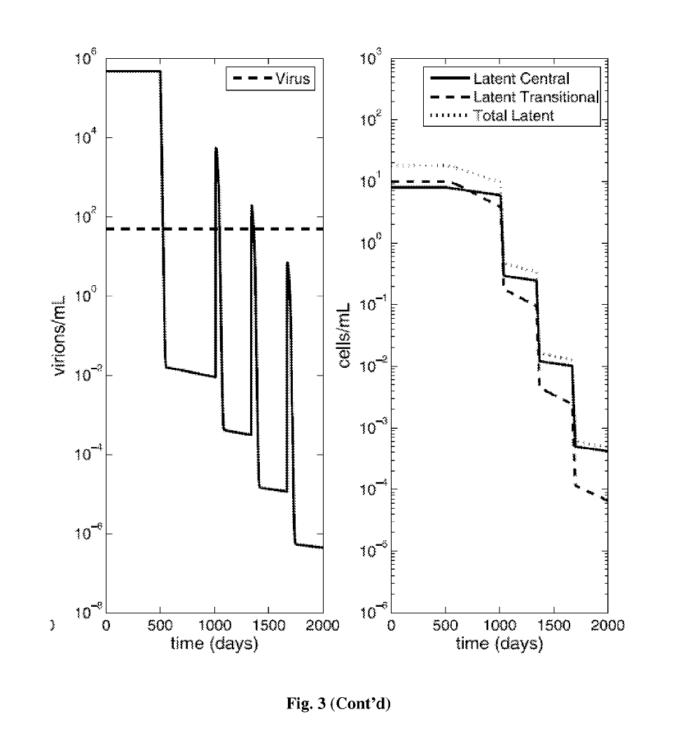

This is modeled by increasing the parameter a to 0.1 per day for a fixed period. Simultaneously, β and d are increased to β′ and d′, respectively, as discussed in Sect. 3.1. This value for a is an order of magnitude larger than the highest value of a describing transient viral blips, since viral blips activate only the portion of the memory class with a particular specificity, while IAT activates a broad section of the latent pool. The modeled therapy regime consists of three activation phases and three relaxation phases. HAART drugs are still administered during all phases of IAT. The activation phases consist of 30 days of IAT and HAART beginning at days 1010, 1340, and 1770. The relaxation stages span 300 days of HAART alone beginning at days 1040, 1370, and 1800. During relaxation phases a is returned to background levels (see FIG. 3). Initiation of IAT causes a spike in viral loads as latent cells are activated and move into the infected class following a proliferative phase. These spikes in viral load are quickly controlled due to the continuation of HAART. Activation causes a rapid drop in both the latent central and transitional memory compartments (see FIG. 3, right panel).

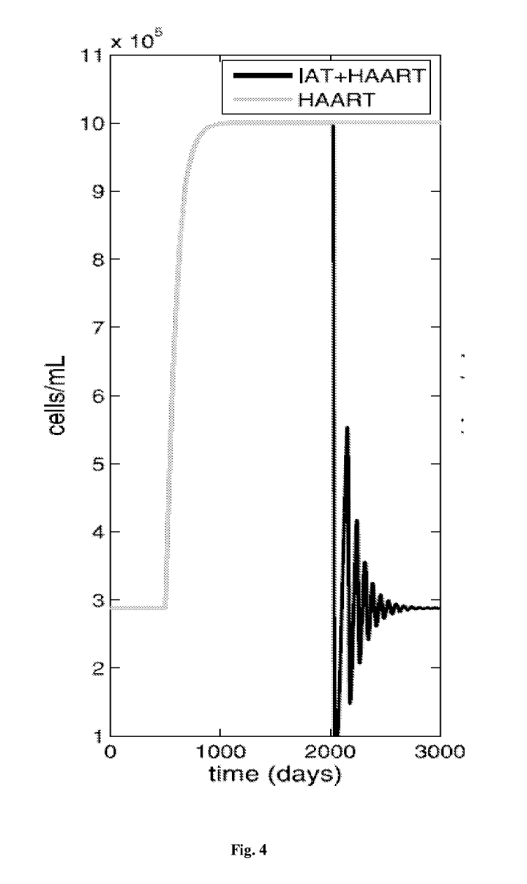

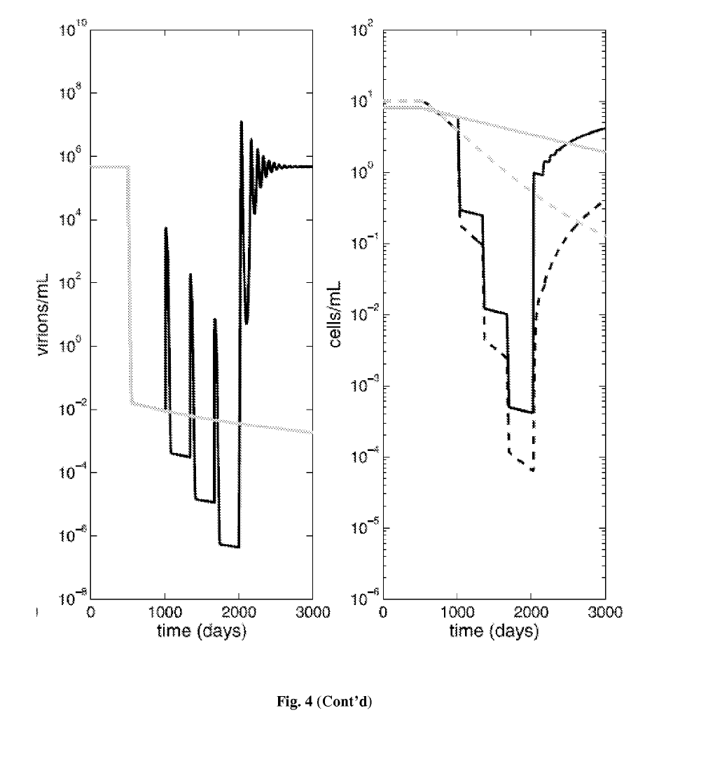

To determine the effect of IAT, we compared the dynamics of latent reservoirs during combination of IAT and HAART treatments with those undergoing HAART alone (see FIG. 4). The addition of IAT lowers viral loads during the relaxation periods between rounds of activation and significantly reduces the size of the latent pool. However, this reduction of the pools of infected cells is not sufficient to allow the cessation of HAART. If HAART is removed (see FIG. 4, days 2000 to 3000) viral loads quickly recover (center panel), CD4 T cell counts are driven down to pretreatment levels (left panel), and the latent pool is reestablished (right panel).

For this reason, this model predicts that IAT is useful primarily to reduce latent HIV-1 reservoirs, and should not be used to allow antiretroviral drug holidays.

3.3.2 Antiproliferation Therapy

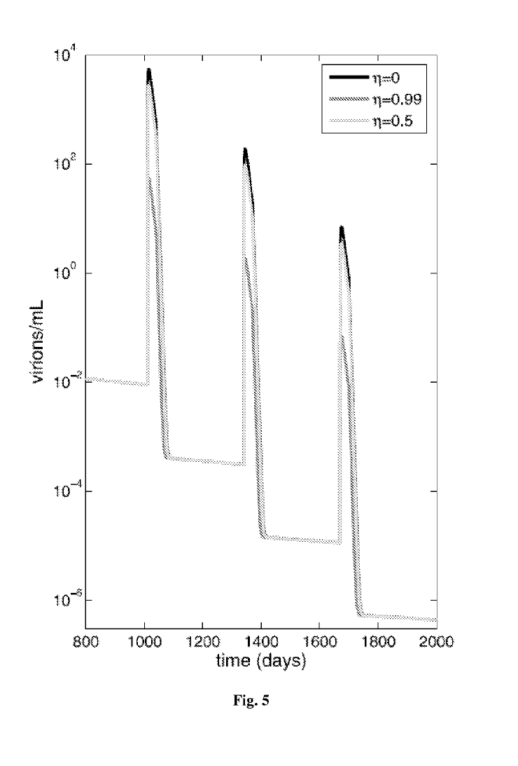

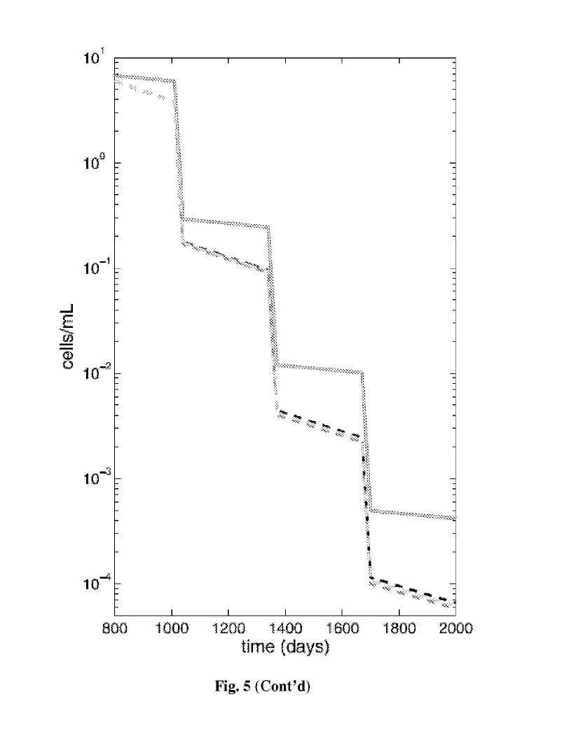

The increased viral load and availability of target cells associated with immune activation therapy raise concerns about increased viral replication and the possible emergence of an escape mutant if HAART wanes over time. One proposed mechanism to reduce the size of the viral spikes is the administration of antiproliferative drugs. In our model, the effects of anti-proliferation drugs are seen as a reduction of the parameters ρ (proliferative expansion of reactivated memory cells) and α (homeostatic proliferation rate of transitional memory cells) by a factor η, where η is the efficacy of the drug. The addition of strong antiproliferation therapy with 99% efficacy reduces the size of viral spikes to the extent that they never surpass the limits of detection, and are therefore no worse than the random activation events which cause viral blips (FIG. 5, left panel). There is no appreciable effect on the dynamics of latent reservoir reduction (FIG. 5, right panel). If no antiproliferative drug effect is included, or if its efficacy is only 50%, the viral spikes reach levels above the 50 copies per ml limit of detection.

### 4 Discussion and Conclusions

Despite the availability of highly effective antiretroviral drugs, the eradication of HIV-1 from an infected individual remains out of reach. One barrier to eradication is the existence of latent pools of infected cells which decay very slowly, even when viral loads remain below the limits of detection for long periods of time. The mathematical model described herein explores the ability of immune activation therapy to speed the elimination of the most prevalent latent pool, memory CD4 T cells. This model investigated the dynamics of two classes of latently infected memory CD4 T cells: central and transitional memory, which were assumed to have average half-lives of 44 and 6 months, respectively.

By separating the memory phenotypes, the observation in Chomont et al. (2009) that central and transitional memory cells are predominant in patients with relatively high and low CD4 counts, respectively, was recovered. After initiation of efficient HAART therapy, the latently infected pool decays at a very slow rate due to the long half-life of memory cells, ranging between 6 and 44 months (Finzi et al. 1997; Siliciano et al. 2003; Chun et al. 2007; Ramratnam et al. 1999; Zhang et al. 1999). This model correlates the large variation in the overall latent cell half-life estimates with the phenotype composition of the latent pool at the start of the treatment. In the absence of nonspecific immune activation, a longer overall half-life of latent reservoir of 27 months is predicted when the central memory class is dominant at the start of HAART and a shorter overall half-life of 14 months when the transitional memory class is dominant at the start of HAART. To investigate speeding latent reservoir elimination, the effect of nonspecific immune activation therapy initiated after HAART to successfully reduce HIV-1 below limits of detection in the blood was modeled. Several strategies currently under investigation look at the possibility of activating latently infected memory cells, while relying on effective HAART to control the spread of viral infection (Archin and Margolis 2006).

Activation exposes these cells to removal by the immune system and death due to the cytopathic effects of viral production. Nonspecific activation strategies have drawback, however, due to the negative effects of broad activation of resting cells, and the potential that some therapies such as the cytokine IL7, may increase the proliferation of latently infected cells (Chomont et al. 2009). An alternative approach is inducing increased expression of HIV-1-specific genes by latently infected cells, exposing them to deletion by the immune system. Histone deacetylase inhibitors (HDACi) such as valproic acid (Archin et al. 2008; Siliciano et al. 2007) and Vorinostat (Contreras et al. 2009) are currently under investigation, along with certain cellular transcription factors (Yang et al. 2009). However, no HIV-1-specific therapies are available to either induce activation or increase gene expression.

Our modeling effort focused on the consequences of unspecified broad activation strategies. The model predicts that immune activation therapy combined with successful HAART significantly reduces the time required to eradicate these latent reservoirs compared with HAART treatment alone, with both latent phenotypes decreasing at the same rate. While it is impossible for our ordinary differential equations model to precisely predict when the number of latently infected cells will reach 0, we consider eradication to be equivalent to a total of fewer than one latently infected memory T cell in a host. As cell populations are measured per milliliter of serum, and we assume HIV-1 is distributed throughout the 15 liters of extracellular fluid, the infected cell concentration corresponding to eradication is 7×10⁻⁵ cells/mL (Callaway and Perelson 2002; Rong and Perelson 2009c).

Our model predicts that under combined IAT and HAART, a treatment time of 4 months of immune activation therapy is required to eradicate latent cells. In contrast, 50 years of HAART of the same efficacy are needed to reach this threshold. These results are highly dependent on the choice of parameter values and on the intensity of the activation a.

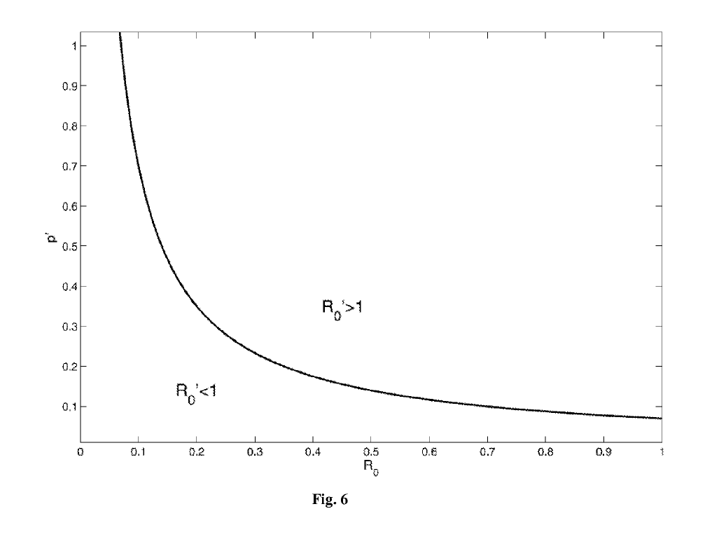

Our model predicts that increased specificity of latent reactivation causes the latent pool to shrink at the same rate as broader reactivation, with less chance for immune-mediated symptoms. The most ideal possible case would be drug-induced HIV-1-specific reactivation of latently infected cells. Our model demonstrates that increased overall nonspecific activation of resting cells (p′>p/R0) can cause HAART failure and reemergence of infection (see FIG. 6). For lower levels of activation, HAART is sufficient to control the spike in viral titers, and the size of the latent pool shrinks at a rate equal to the rate at which latently infected cells are activated. In this case, the extent of resting cell activation is still significant in terms of patient outcome, as broad activation can result in immune-mediate pathology (Suntharalingam et al. 2006).

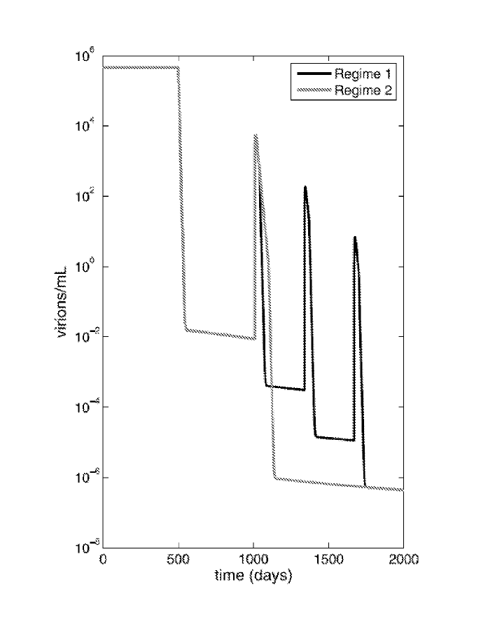

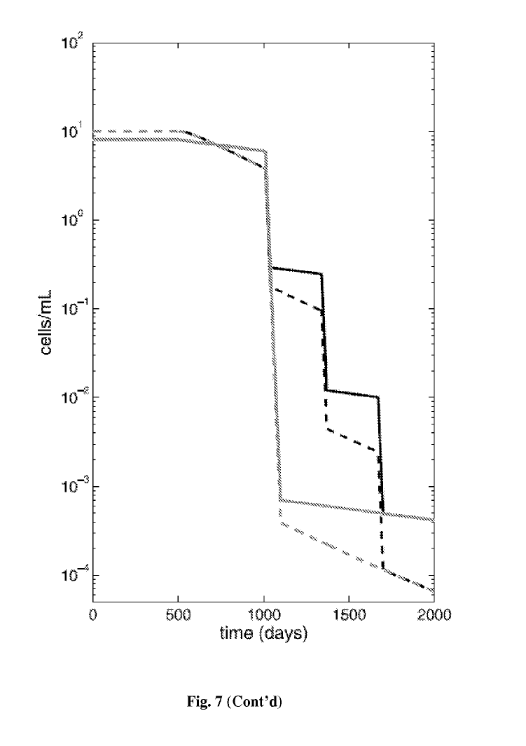

As the specificity of the reactivation increases, the negative side effects of reactivation decrease, while the rate at which the latent pool is depleted remains constant at the activation rate a. An important feature of immune activation therapy as described by this model is the additivity of treatment effects. Over 990 days, the same reduction of the latent reservoir is achieved by a regimen of 90 days of combined IAT and HAART followed by 900 days of HAART alone and by three cycles of 30 days of combined IAT and HAART and 300 days of HAART (see FIG. 7).

This allows for flexibility in optimizing the treatment schedule to reduce the negative side effects of broad immune activation. Although IAT reduces the size of the latently infected memory pool, activating these cells leads to spikes in free virus concentration. If not controlled by antiretroviral therapy, this transient viremia increases the risk that drug-resistant mutants strains of HIV-1 could arise due to increased viral replication.

Viral recurrence is limited by considering the effects that antiproliferation drugs have on blocking the proliferation of newly activated cells and, as a result, on lowering the magnitude of viral spikes caused by broad immune activation. If such a drug is 99% effective, then the viral spikes are kept below the limits of detection. While antiproliferation therapy carries the potential risks of reducing overall T cell survival and function, other therapies that induce HIV-1 expression in latently infected resting CD4+ T-cells may allow for the recognition of latent cell by the immune system in the absence of cell activation (Margolis 2010; Wolschendorf et al. 2010; Mellberg et al. 2011; Kovochich et al. 2011).

This model considers the simplified assumption that the class of uninfected cells accounts for both activated and resting CD4 T cells, with the fraction in the activated class being absorbed into the death and infectivity rates. The overall results do not change when the model is expanded to account for these populations independently (results not shown). Moreover, the model does not consider other possible latent pools of infection, such as macrophages or dendritic cells. In order for eradication of HIV-1 to be achieved, all latent pools would have to be eliminated. Immune activation therapy could aid in the removal of one the barriers to eventual overall clearance of the virus. This requires the assumption that antiretroviral therapy is effective enough that there are no persistent active sources of new virions.

While the continuation of highly effective HAART for long periods could lead to clearance of HIV-1 by suppressing the source of new infected cells, time is a crucial factor. Current antiretrovirals have significant side effects (d'Arminio Monforte et al. 2000; Blas-Garcia et al. 2011), and the stability of latent infection reservoirs makes the waiting time for clearance too long to be practical. The addition of immune activation therapy to viral suppression drugs represents a step toward shortening the treatment time needed, and helps reveal other barriers that still stand in the way of a cure for HIV-1 infection.

The variables and parameter values used in this model are presented below in Table 1:

TABLE 1
Variables and parameter values used for simulations

| | DESCRIPTION | VALUES | | REFERENCES |
|---|---|---|---|---|
| VARIABLE | | | | |
| T | Target cells (mostly CD4 T | — | | — |
| | cells) | | | |
| I | Actively infected CD4 T cells | — | | — |
| V | HIV virus | — | | — |
| L_{C} | Central memory latently | | | — |
| | infected CD4 T cells | | | |
| L_{T} | Transitional memory latently | — | | — |
| | infected CD4 T cells | | | |
| PARAMETER | | | | |
| s | Target cell production rate | 2.12 × 10⁴ | cells mL⁻¹ day⁻¹ | Sachsenberg et al. (1998) |
| b_{T} | T cell density for half-maximal | 3 × 10⁵ | cells mL⁻¹ | See text |
| | target expansion rate | | | |
| d | Target cells death rate | 0.0163 | day⁻¹ | See text |
| ε | HAART efficacy | 0.99 | | Shen and Siliciano (2008) |
| β | Infection rate | Varies (see text) | | Stafford et al. (2000) |
| f | Fraction resulting in latent | | 1.5 × 10⁻⁶ | Chun et al. (1997b) |
| | infections | | | |
| δ | Death rate of productively | 0.39 | day⁻¹ | Markowitz et al. (2003) |
| | infected cells | | | |
| N | Burst size | 2000 | | Hockett et al. (1999) |
| c | Virus clearance rate | 23 | day⁻¹ | Ramratnam et al. (1999) |
| θ | Transition from central to | 10² | cells mL⁻¹ day⁻¹ | See text |
| | transitional memory class | | | |
| b_{L} | T cell density for half-maximal | 3 × 10⁵ | cells mL⁻¹ day⁻¹ | See text |
| | latent expansion rate | | | |
| α | Homeostatic proliferation rate | 2.7 × 10³ | cells mL⁻¹ day⁻¹ | See text |
| a | Latent cells activation rate | Varied | | See text |
| ρ | Proliferation factor of activated | 100 | | See text |
| | cells | | | |
| d_{C} | Death rate of central memory | 5 × 10⁻⁴ | day⁻¹ | Siliciano et al. (2003); |
| | cells | | | Finzi et al. (1999) |
| d_{T} | Death rate of transitional | 4 × 10⁻³ | day⁻¹ | Chun et al. (2007); |
| | memory cells | | | Zhang et al. (1999) |

| | | | |
|---|---|---|---|
| η | Anti-proliferation efficacy | 0.99 | See text |
| p | Fraction of activated | 0.07 | Hunt et al. (2008) |
| | uninfected CD4 T cells | | |

### REFERENCES

Archin, N. M., & Margolis, D. M. (2006). Attacking latent HIV provirus: from mechanism to therapeutic strategies. Curr. Opin. HIV & AIDS, 1, 134-140.
Archin, N. M., et. al. (2008). Valproic acid without intensified antiviral therapy has limited impact on persistent HIV infection of resting CD4+ T cells. AIDS, 22, 1131-1135.
Blankson, J. N., et. al. (2002). The challenge of viral reservoirs in HIV-1 infection. Annu. Rev. Med., 53(1), 557-593.
Blas-Garcia, A., et. al. (2011). Twenty years of HIV-1 non-nucleoside reverse transcriptase inhibitors: time to reevaluate their toxicity. Curr. Med. Chem., 18(14), 2186-2195.
Burnett, J. C., et. al. (2010). Combinatorial latency reactivation for HIV-1 subtypes and variants. J. Virol., 84(12), 5958-5974.
Callaway, D. S., & Perelson, A. S. (2002). HIV-1 infection and low steady state viral loads. Bull. Math. Biol., 64(1), 29-64.
Chomont, N., et. al. (2009). HIV reservoir size and persistence are driven by T cell survival and homeostatic proliferation. Nat. Med., 15, 893-900.
Chomont, N., et. al. (2011). Maintenance of CD4+ T-cell memory and HIV persistence: keeping memory, keeping HIV. Curr. Opin. HIV & AIDS, 6, 30-36.
Chun, T. W., et. al. (1997a). Quantification of latent tissue reservoirs and total body viral load in HIV-1 infection. Nature, 387, 183-188.
Chun, T. W., et. al. (1997b). Presence of an inducible HIV-1 latent reservoir during highly active antiretroviral therapy. Proc. Natl. Acad. Sci. USA, 94(24), 13193-13197.
Chun, T. W., et. al. (1998). Early establishment of a pool of latently infected, resting CD4+ T cells during primary HIV-1 infection. Proc. Natl. Acad. Sci. USA, 95(15), 8869-8873.
Chun, T. W., et. al. (1999). Effect of interleukin-2 on the pool of latently infected, resting CD4+ T cells in HIV-1-infected patients receiving highly active antiretroviral therapy. Nat. Med., 5(6), 651-655.
Chun, T. W., et. al. (2007). Decay of the HIV reservoir in patients receiving antiretroviral therapy for extended periods: implications for eradication of virus. J. Infect. Dis., 195, 1762-1764.
Contreras, X., et. al. (2000). Insights into the reasons for discontinuation of the first highly active antiretroviral therapy (HAART) regimen in a cohort of antiretroviral naive patients. AIDS, 14, 499-507.
Davey, R. T., et. al. (1997). Subcutaneous administration of interleukin-2 in human immunodeficiency virus type 1-infected persons. J. Infect. Dis., 175(4), 781-789.
Di Mascio, M., et. al. (2003). In a subset of subjects on highly active antiretroviral therapy, human immunodeficiency virus type 1 RNA in plasma decays from 50 to <5 copies per milliliter, with a half-life of 6 months. J. Virol., 77, 2271-2275.
Di Mascio, M., et. al. (2005). Duration of an intermittent episode of viremia. Bull. Math. Biol., 67, 885-900.
Dornadula, G., et. al. (1999). Residual HIV-1 RNA in blood plasma of patients taking suppressive highly active antiretroviral therapy. JAMA J. Am. Med. Assoc., 282(17), 1627-1632.
Finzi, D., et. al. (1997). Identification of a reservoir for HIV-1 in patients on highly active antiretroviral therapy. Science, 278(5341), 1295-1300.
Finzi, D., et. al. (1999). Latent infection of CD4+ T cells provides a mechanism for life long persistence of HIV-1, even in patients on effective combination therapy. Nat. Med., 5, 512-517.
Fraser, C., et. al. (2000). Reduction of the HIV-1-infected T-cell reservoir by immune activation treatment is dose-dependent and restricted by the potency of antiretroviral drugs. AIDS, 14, 659-669.
Freed, E. O., & Martin, M. A. (2007). HIVs and their replication. In D. M. Knipe & P. M. Howley (eds.), Field's Virology, pp. 2107-2186.
Wolters Kluwer Health/Lippincott Williams & Wilkins, Philadelphia Geeraert, L., Kraus, G., & Pomerantz, R. J. (2008). Hide-and-seek: The challenge of viral persistence in HIV-1 infection. Annu. Rev. Med., 59(1), 487-501.
Gunthard, et. al. (2001). Residual human immunodeficiency virus (HIV) type 1 RNA and DNA in lymph nodes and HIV RNA in genital secretions and in cerebrospinal fluid after suppression of viremia for 2 years. J. Infect. Dis., 183(9), 1318-1327.
Harrigan, P. R., et. al. (1999). Rate of HIV-1 RNA rebound upon stopping antiretroviral therapy. AIDS, 13, F59-F62.
Ho, D. D., et. al. (1995). Rapid turnover of plasma virions and CD4 lymphocytes in HIV-1 infection. Nature, 373, 123-126.
Hockett, R. D et. al. (1999). Constant mean viral copy number per infected cell in tissues regardless of high, low, or undetectable plasma HIV RNA. J. Exp. Med., 17, 1545-1554.
Hunt, P. W., et. al. (2008). Relationship between T cell activation and CD4+ T cell count in HIV-seropositive individuals with undetectable plasma HIV RNA levels in the absence of therapy. J. Infect. Dis., 197(1), 126-133.
Jones, L. E., & Perelson, A. S. (2007). Transient viremia, plasma viral load, and reservoir replenishment in HIV-1 infected patients on antiretroviral therapy. J. Acquir. Immune Defic. Syndr., 45, 483-493.
Kaufmann, G. R., et. al. (2000). Long-term immunological response in HIV-1-infected subjects receiving potent antiretroviral therapy. AIDS, 14(8), 959-969.
Kim, H., & Perelson, A. S. (2006). Viral and latent reservoir persistence in HIV-1 infected patients on therapy. PLoS Comput. Biol., 2, e135. Kovochich, M., Marsden, M. D., & Zack, J. A. (2011). Activation of latent HIV using drug-loaded nanoparticles. PLoS ONE, 6, e18270.
Lehrman, G., et. al. (2005). Depletion of latent HIV-1 infection in vivo: a proof-of-concept study. The Lancet, 366(9485), 549-555.
Lok, J. J., et. al. (2010). Long-term increase in CD4+ T-cell counts during combination antiretroviral therapy for HIV-1 infection. AIDS, 24(12), 1867-1876.
Margolis, D. M. (2010). Mechanisms of HIV latency: an emerging picture of complexity. Curr. HIV/AIDS Rep., 7, 37-43.
Markowitz, M., et. al. (2003). A novel antiviral intervention results in more accurate assessment of human immunodeficiency virus type 1 replication dynamics and T-cell decay in vivo. J. Virol., 77, 5037-5038.
Mellberg, T., et. al. (2011). Rebound of residual plasma viremia after initial decrease following addition of intravenous immunoglobulin to effective antiretroviral treatment of HIV. AIDS Res. Ther., 8(1), 21.
Nettles, R. E., et. al. (2005). Intermittent HIV-1 viremia (blips) and drug resistance in patients receiving HAART. JAMA J. Am. Med. Assoc., 293(7), 817-829.
Palmer, S., et. al. (2008). Low-level viremia persists for at least 7 years in patients on suppressive antiretroviral therapy. Proc. Natl. Acad. Sci. USA, 105(10), 3879-3884.
Perelson, A. S., et. al. (1997). Decay characteristics of HIV-1-infected compartments during combination therapy. Nature, 387, 188-191.
Ramratnam, B., et. al. (1999). Rapid production and clearance of HIV-1 and hepatitis C virus assessed by large volume plasma apheresis. Lancet, 354, 1782-1785.
Rong, L., & Perelson, A. S. (2009a). Modeling HIV persistence, the latent reservoir, and viral blips. J. Theor. Biol., 260(2), 308-331.
Rong, L., & Perelson, A. S. (2009b). Asymmetric division of activated latently infected cells may explain the decay kinetics of the HIV-1 latent reservoir and intermittent viral blips. Math. Biosci., 217(1), 77-87. Mathematical Models of Inflammation.
Rong, L., & Perelson, A. S. (2009c). Modeling latently infected cell activation: Viral and latent reservoir persistence, and viral blips in HIV-1 infected patients on potent therapy. PLoS Comput. Biol., 5, e1000533. Sachsenberg, N., Perelson, A. S., Yerly, S., Schockmel, G. A., Leduc, D., Hirschel, B., & Perrin, L. (1998). Turnover of CD4 and CD8 T lymphocytes in HIV-1 infection as measured by Ki-67 antigen. J. Exp. Med., 187, 1295-1303.
Sallusto, F., Geginat, J., & Lanzavecchia, A. (2004). Central memory and effector memory T cell subsets: Function, generation, and maintenance. Annu. Rev. Immunol., 22, 745-763. Sedaghat, A. R., Siciliano, R. F., & Wilke, C. O. (2008). Low-level HIV-1 replication and the dynamics of the resting CD4+ T cell reservoir for HIV-1 in the setting of HAART. BMC Infect. Dis., 8, 1-14.
Shen, L., & Siliciano, R. F. (2008). Viral reservoirs, residual viremia, and the potential of highly active antiretroviral therapy to eradicate HIV infection. J. Allergy Clin. Immunol., 122, 22-28.
Siliciano, J. D., et. al. (2003). Long-term follow-up studies confirm the stability of the latent reservoir for HIV-1 in resting CD4+ T cells. Nat. Med., 9, 727-728.
Siliciano, J. D., et. al. (2007). Stability of the latent reservoir for HIV-1 in patients receiving valproic acid. J. Infect. Dis., 195, 833-836.
Simon, V., & Ho, D. D. (2003). HIV-1 dynamics in vivo: implications for therapy. Nat. Rev., Microbiol., 1, 181-190.
Smith, R., & Aggarwala, B. (2009). Can the viral reservoir of latently infected CD4 T cells be eradicated with antiretroviral HIV drugs? J. Math. Biol., 59, 697-715.
Stafford, M. A., et. al. (2000). Modeling plasma virus concentration during primary HIV infection. J. Theor. Biol., 203, 285.
Suntharalingam, G., et. al. (2006). Cytokine storm in a phase 1 trial of the anti-CD28 monoclonal antibody TGN1412. N. Engl. J. Med., 355, 1018-1028.
Wei, X., et. al. (1995). Viral dynamics in human immunodeficiency virus type 1 infection. Nature, 373, 117-122.
Wolschendorf, F., et. al. (2010). Hit-and-run stimulation: a novel concept to reactivate latent HIV-1 infection without cytokine gene induction. J. Virol., 84(17), 8712-8720.
Yang, H. C., et. al. (2009). Isolation of a cellular factor that can reactivate latent HIV-1 without T cell activation. Proceedings of the National Academy of Science (USA) (pp. 6321-6325).
Yates, A., et. al. (2007). Understanding the slow depletion of memory CD4+ T cells in HIV infection. PLoS Med., 4, e177.
Zhang, L., et. al. (1999). Quantifying residual HIV-1 replication in patients receiving combination antiretroviral therapy. N. Engl. J. Med., 340, 1605-1613.

### Example 2

### Determining the Effectiveness of a Given Radioimmunotherapy Treatment

The following prophetic example shows ways to determine whether a given radioimmunotherapy will be effective for use in the methods described herein.

Materials and Methods

Antibodies.

Goat polyclonal antibody (Ab) against gp-120 (IgG1) can be purchased from Biodesign International (Saco, Me.). Murine 18B7 monoclonal antibody (mAb) (IgG1) specific for cryptococcal polysaccharide (Casadevall et al., 1998) can be used as an isotype-matching control. As described in U.S. Pat. No. 5,731,189, lymphoblastoid cell line 126-6 producing human monoclonal antibodies directed against gp41 is deposited with the American Type Culture Collection (10801 University Boulevard, Manassas, Va. 21110-2209) on Feb. 24, 1989 and received ATCC Accession number CRL 10037. Human mAb 1418 (IgG1) to parvovirus Bl 9 (Gigler et al., 1999) can be used as an irrelevant control for mAb 246D, and human mAb 447 (IgG3) to the V3 loop of HIV-I gp120 (Conley et al., 1994) can be used as a positive control in the FACS studies. Prior to use, the antibodies can be purified by affinity chromatography.

Radioisotopes and Quantification of Radioactivity.

¹⁸⁸-Re in the form of Na perrhenate (Na188ReO4) can be eluted from a 188W/188Re generator (Oak Ridge National Laboratory (ORNL), Oak Ridge, Tenn.). Actinium-225 (225Ac) for construction of a ²²⁵Ac/²¹³Bi generator can be acquired, for example, from the Institute for Transuranium Elements, Karlsruhe, Germany. A Ac/Bi generator can be constructed using MP-50 cation exchange resin, and Bi can be eluted with 0.15 M HI (hydroiodic acid) in the form of ²¹³-Bi/I52″ as described in Boll R A, Mirzadeh S, and Kennel S J. Optimizations of radiolabeling of immuno-proteins with 213-Bi. Radiochim. Acta 79: 145-149, 1997. A gamma counter (Wallac) with an open window can be used to count the ¹⁸⁸-Re and ²¹³-Bi samples.

Radiolabeling of Antibodies with ¹⁸⁸-Re and ²¹³-Bi.

Antibodies can be radiolabeled with beta-emitter ¹⁸⁸-Re (half-life 17.0 h) or alpha-emitter ²¹³-Bi (half-life 45.6 min). Abs can be labeled “directly” with ¹⁸⁸-Re via reduction of antibody disulfide bonds by incubating the antibody with 75-fold molar excess of dithiothreitol (Dadachova, E., Mirzadeh, S., Smith, S. V., Rnapp, F. F., and Hetherington, E. L. Radiolabelling antibodies with 166-Holmium. Appl. Rad. Isotop. 48: 477-481, 1997) for 40 niin at 37° C. followed by centrifugal purification on Centricon-30 or -50 microconcentrators with 0.15 M NH4OAc, pH 6.5. Simultaneously 3-10 mCi (110-370 MBq)¹⁸⁸-ReO4″ in saline can be reduced with SnCl₂ by incubation in the presence of Na gluconate, combined with purified reduced antibodies and kept at 37° C. for 60 min. Radioactivity not bound to the antibody can be removed by centrifugal purification on Centricon microconcentrators.

For radiolabeling with ²¹³-Bi, Abs can be conjugated to bifunctional chelator N-[2-amino-3-(p-isothiocyanatophenyl)propyl]-trans-cyclohexane-1,2-diamine-N,N′,N″,N″′,N″″-pentaacetic acid (CHXA″) as in Boll et al, 1997, supra; Chappell L L, Dadachova E, Milenic D E, Garmestani K, Brechbiel M W. Synthesis and Characterization of a Novel Bifunctional Chelating Agent for Lead(II). Conjugation to a Monoclonal Antibody, Radiolabeling with Lead-203 and Serum Stability Determination, Nucl. Med. Biol. 27: 93-100, 2000; Dadachova et al., 1997, supra. The average final number of chelates per antibody molecule can be determined by the Yttrium-Arsenazo III spectrophotometric method (Pippin et al., 1992). CHXA″-conjugated Abs can be radiolabeled with ²¹³-Bi by incubating them for 5 min with BiI₅″ at room temperature. If required, the radiolabeled antibodies can be purified by size exclusion HPLC (TSK-Gel® G3000SW, TosoHaas, Japan).

Effectiveness of the Radiolabeled Antibodies Against HIV-Infected Cells

In Vitro Killing of ACH-2 Cells.

An ACH-2 cell line, a latent T-cell clone infected with HIV-IIIB that produces steady low levels of supernatant RT and p24, can be obtained through the NIH AIDS Research and Reference Reagent Program, Division of AIDS, NIAID, NIH: ACH-2, catalogue #349 from Dr. Thomas Folks.

HIV-I chronically infected human T-cells ACH-2 (phytohaemagglutinin (PHA)-stimulated, phorbol myristate (PMA)-stimulated, and non-stimulated) can be treated with 0-50 μCi of radiolabeled Abs, or with matching amounts (2.5-12.5 μg) of “cold” Abs. In one embodiment, approximately 2×10⁵ cells per sample can be used. The cells can be incubated with radiolabeled or “cold” Abs at 37° C. for 3 h, transferred into fresh cell culture medium and then incubated in 5% CO₂ at 37° C. for 72 h. The number of viable cells 72 h post-treatment can be assessed using a blue dye exclusion assay.

Treatment of HIV1-Infected and Non-Infected Peripheral Blood Mononuclear Cells (PBMCs) with Radiolabeled mAbs.

Human Peripheral Blood Mononuclear Cells (PBMCs) obtained from the New York Blood Center (NY, N.Y.) can be stimulated with PHA and interleukin-2 (IL-2) 48 h prior to infection with HIV-I strain JR-CSF (obtained through the NIH AIDS Research and Reference Reagent Program, Division of AIDS, NIAID, NIH: HIV-1jR-csF, catalogue #394 from Dr. Irvin S. Y. Chen).

While the number of ACH-2 cells infected with HIV-I can be almost 100%, only a fraction (around 10-30%) of the PBMCs are typically infected with HIV-I, as determined by limiting dilution co-culture technique (Ho, D. D., Moudgil, T. & Alam, M. Quantitation of human immunodeficiency virus type 1 in the blood of infected persons. N. Engl. J. Med. 321: 1621-1625, 1989). Cells exposed to HIV-I are referred to as “infected” cells, and those which were not exposed to the virus are referred to as “non-infected” cells. At 48 h after infection with HIV-I, infected PBMCs can be treated with 0-20 μCi of the putative radiolabeled antibodies or with matching amounts (2.5-12.5 μg) of “cold” Abs. In one embodiment, approximately 2×10⁵ cells per sample can be used. As controls, non-infected PBMCs can be treated with ²¹³-Bi-anti-gp41 mAb. The cells can be incubated with radiolabeled or “cold” Abs at 37° C. for 3 h, transferred into fresh cell culture medium and then incubated in 5% CO₂ at 37° C. for 72 h. The number of viable cells 72 h post-treatment can be assessed using a blue dye exclusion assay.

Flow Cytometric Analysis of mAbs Binding to Virus Infected Cells.

Binding studies of human mAbs to the surface of hPBMCs infected with the JR-CSF strain of HIV-I can be performed as described previously (Zolla-Pazner et al, 1995). Briefly, PHA-stimulated hPBMCs can be infected with 1 ml of stock HIV-1JR.CSF virus and cultured for 13 days in medium supplemented with human recombinant IL-2 (20 U/ml, Boehringer Mannheim Biochemicals, Indianopolis, Ind.). The cells can be incubated with each human mAb at 10 μg/ml for 1 h on ice, washed and reincubated with PE-labeled goat F(ab′)2 anti-human IgG(γ) (Caltag Laboratories, Burlingame, Calif.). Using a FACScan flow cytometer (Becton Dickinson), live lymphocytes can be selected for analysis by gating with forward and 90° scatter. The negative control can consist of cells from infected cultures stained with the conjugated anti-IgG in the absence of a human mAb.

Treatment of HIV Infected PBMCs Pre-Incubated with HTV Positive Blood.

Human PBMCs can be grown and infected with JR-CSF strain of HIV-1 as described above. Infected PBMCs, for example, a sample size of around 2×10⁵ cells, can be incubated for 1 h at 37° C. with 200 μL of undiluted serum from a HIV 1-positive patient, or with the same volume of 1:10 or 1:100 diluted HIV 1-positive serum using HIV-negative serum as a diluent, or with HIV-negative serum only. Following the incubation the cells can be washed with PBS, 1 mL PBS per sample can be added and the cells can be treated with a suitable amount, such as 20 μCi, or a radiolabeled antibody or left untreated. The cells can be incubated with radiolabeled mAb at 37° C. for 3 h, transferred into fresh cell culture medium and then incubated in 5% CO₂ at 37° C. for 72 h. The number of viable cells 72 h post-treatment can be assessed using blue dye exclusion assay.

Treatment of Naked HIV1 Virus with Radiolabeled Anti-Gp41 mAb.

Viral particles can be incubated with mAbs for 3 h, followed by infection of healthy PBMCs. On Day 6 post-infection, the cultures can be analyzed for the presence of HIV core protein p24 by core Profile ELISA (DuPont-NEN).

Determination of Splenic Uptake of Radiolabeled mAbs.

Two groups of SCID mice can be used in this experiment. One group can be injected intrasplenically with HIV-I infected PBMCs and a second group can be injected with non-infected PBMCs (25 million cells per mouse). One hour later a suitable amount, such as 20 μCi (20 μg) of a radiolabeled antibody can be given IP to each mouse. Three hours post-injection, the animals can be sacrificed, their spleens removed, blotted from blood, weighed, counted in a gamma counter, and the percentage of injected dose per gram (JDIg) can be calculated.

Determination of Platelet Counts in Mice Treated with Radiolabeled mAbs.

Platelet counts can be used as a marker of RIT toxicity in treated animals. For measurement of platelet counts, the blood of SCID mice injected intrasplenically with HIV-I-infected hPBMCs and either treated with a suitable amount (such as 100 μCi (20 μg)) of a radiolabeled antibody, IP, 1 hour after infection with PBMCs or untreated can be collected from the tail vein into 200 μL 1% ammonium oxalate on day 0, 4, 8 and 15 days post-therapy. Platelets can be counted in a hemocytometer, using phase contrast, at 400 times magnification, as described in Miale, J. B. Laboratory Medicine Hematology, The CV Mosby Company, St. Louis, Mo., p. 864, 1982.

Treatment of HIV 1-Infected Mice with Radiolabeled mAbs.

Human PBMCs can be stimulated with PHA and IL-2 48 h prior to infection with HIV-I strain JR-CSF. At 48 h after infection with HIV-I, infected PBMCs can be injected intrasplenically (25 million cell per animal) into groups of SCID mice (10 mice per group). Mice can receive either 20 μg “cold” antibody, or radiolabeled antibody, and isotype-matching controls can be used. 1 hour after infection with PBMCs, the antibodies are administered IP. In some experiments mice can be given 80 μCi (20 μg) of the antibodies IP 1 h prior to infection with PBMCs. The SCID mice can be sacrificed 72 hours after treatment and the spleens can be harvested and processed. A limiting dilution co-culture of the splenocytes can be performed using freshly activated PBMCs as described in Wang E J, Pettoello-Mantovani M, Anderson C M, Osiecki K, Moskowitz D, Goldstein H. Development of a novel transgenic mouse/SCID-hu mouse system to characterize the in vivo behavior of reservoirs of human immunodeficiency virus type 1-infected cells. J Infect Dis. 186(10):1412-21, 2002). Supernatants can be harvested on day 8 after initiation of co-culture and analyzed for the presence of HIV-I core protein p24 by core Profile ELISA (DuPont-NEN). Data can be reported as infected splenocytes/10⁶ splenocytes. The number of HIV-I-infected cells present in the spleen can be measured using limiting dilution quantitative co-culture as described by Ho et al. (1989), supra. This technique measures the number of cells capable of producing infectious HIV-I. Five-fold dilutions of cells isolated from each spleen (in the range 1×10⁶-3.2×10² cells) can be cultured in duplicate at 37° C. in 24-well culture plates with PHA-activated hPBMCs (1×10⁶ cells) in 2.0 mL of RPMI 1640 medium containing fetal calf serum (10% vol/vol) and interleukin-2 (32 U/mL). The HIV-I p24 antigen content of the supernatant can be measured 1 week later, using the HIV-I p24 core profile ELISA (DuPont-NEN). The lowest number of added cells that infect at least half the duplicate cultures with HIV-I can be determined and represent the frequency of cells productively infected with HIV-I in each spleen, reported as TCID5Q/10⁶ splenocytes. In dose response experiments, the groups of infected mice can be given 40, 80 or 160 μCi (20 μg) of a radiolabeled antibody, 20 μg “cold” antibody, or left untreated, and the efficacy of the therapy can be assessed.

Statistical Analysis.

Prism software (GraphPad, San Diego, Calif.) can be used for statistical analysis of the data. Student's t-test for unpaired data can be employed to analyze differences in the number of viable ACH-2 cells, PBMCs or infected splenocytes/10⁶ splenocytes between differently treated groups during in vitro and in vivo therapy studies, respectively.

In Vitro Killing of HIV-Infected ACH-2 Cells with Radiolabeled mAbs.

To determine the capacity of RIT to kill HIV-I infected cells, an antibody having the properties described herein (i.e., targeting a desired HIV-protein) can be labeled with radioisotopes as described herein. HIV-I-infected ACH-2 cells can be incubated with a desired radiolabeled antibody, ideally with a control (such as ¹⁸⁸-Re-control Ab, which is an irrelevant murine mAb 18B7) or “cold” anti-gp120 Ab.

Active antibodies will show significant killing of HIV-infected ACH-2 cells, whereas the control Ab 188Re-18B7 with the same specific activity should produce only minimal killing within the investigated range of activities (P=0.01). The significantly higher killing associated with the specific antibody will reflect higher radiation exposure for ACH-2 cells as a consequence of Ab binding to a particular protein target (such as the gp120 glycoprotein expressed on the surface of ACH-2 cells). Typically, the antibodies will not kill ACH-2 cells if they are not radiolabeled (i.e., “cold”) antibodies.

This type of analysis can be used to establish the feasibility of targeting particular viral proteins in chronically infected cells with RIT.

It is worthy of note that naked HIV-I virus is typically not the target of radiolabeled antibodies.

Elimination of HIV-I Infected PBMCs in Mice by RIT

246D can be used for in vivo experiments. Targeting gp41 has the advantage that this protein is reliably expressed on the surface of chronically infected cells. In addition to the advantages of using human mAb relative to goat polyclonal sera with regards to specific activity and specificity, published data indicate that immunotoxins are more efficient against HIV-infected cells when delivered to the cells by anti-gp41 mAbs rather than anti-gp120 mAbs (Pincus S H, Fang H, Wilkinson R A, Marcotte T K, Robinson J E, Olson W C. In vivo efficacy of anti-glycoprotein 41, but not anti-glycoprotein 120, immunotoxins in a mouse model of HIV infection. J Immunol. 170(4):2236-41, 2003). In the present mouse model, HIV-infected cells are residing in the spleen, which is one of the significant reservoirs of HIV-harboring cells in humans, and thus this model has advantages over more artificial lymphoma tumor-type models (Pincus et al., 2003, supra).

Human PBMCs infected with HIV-IJR-CSF can be injected into the spleens of SCID mice and the mice treated as indicated. Doses of 80 μCi dose radiolabeled antibodies can be chosen, for example, based on known efficacy in other indications, such as RIT of fungal and bacterial infections (Dadachova E, Bryan R A, Frenkel A, Zhang T, Apostolidis C, Nosanchuk J S, Nosanchuk J D, Casadevall A. Evaluation of acute hematologic and long-term pulmonary toxicities of radioimmunotherapy of Cryptococcus neoformans infection in murine models. Antimicrob Agents Chemother. 48(3): 1004-6, 2004a.

Dadachova E, Burns T, Bryan R A, Apostolidis C, Brechbiel M W, Nosanchuk J D, Casadevall A, Pirofski L. Feasibility of radioimmunotherapy of experimental pneumococcal infection. Antimicrob Agents Chemother. 48(5): 1624-9, 2004b). The mice can be evaluated 72 hours later for the presence of residual HIV-I-infected cells by quantitative co-culture (Conley, A J. et al Neutralization of primary human immunodeficiency virus type 1 isolates by the broadly reactive anti-V3 monoclonal antibody, 447-52D. J Virol. 68: 6994-7000, 1994). The 72 hour time period can be chosen to give sufficient time for radiolabeled antibodies to deliver a lethal dose of radioactivity to the cells. This is particularly true for rhenium-labeled antibodies, as the * Re half-life is 16.9 hr and several half-lives may be required for a given radionuclide to deliver the dose to the target.

To investigate the specificity of radiolabeled mAb binding to gp41 HIV-infected hPBMCs, the splenic uptake of a putative radiolabeled antibody can be compared in mice injected intrasplenically with hPBMCs and HIV-I infected hPBMCs. The uptake can be expressed as percentage of injected dose (ID) per gram of spleen for non-infected and infected PBMCs, respectively. The results can establish the in vivo targeting of radiolabeled antibodies to HIV-I-infected cells.

Lack of Hematological Toxicity of RIT of HIV Infection.

The hematological toxicity of radiolabeled antibodies during HIV-I infection can be evaluated in SCID mice by platelet counts. The platelet count nadir usually occurs 1 week after radiolabeled antibody administration to tumor-bearing animals (Behr et al., 1999; Sharkey et al., 1997).

A lack of hematologic toxicity suggests that the antibodies very specifically target infected PBMCs, since gp41 antigen is only expressed on infected cells in the mouse.

While only a few embodiments of the invention have been shown and described herein, it will become apparent to those skilled in the art that various modifications and changes can be made in the present invention without departing from the spirit and scope of the present invention. All such modification and changes coming within the scope of the appended claims are intended to be carried out thereby. Any patents or publications mentioned in this specification are indicative of levels of those skilled in the art to which the patent or publication pertains as of its date and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material referred to.

1. A method for treating an HIV-1-positive patient, comprising:
a) treating the patient with HAART until viral loads drop below 50 copies/mL;
b) while maintaining HAART, administering
i) prostratin, bryostatin-1, or a prostratin or bryostatin analog and, optionally,
ii) an HDAC inhibitor
for a time between one week and ten weeks,
c) repeating steps a) and b) for a minimum of seven cycles,
d) stopping all therapy, and
e) periodically measuring the number of viral copies in the patient's serum,
wherein if a patient does not have greater than 50 copies/mL after a minimum of two weeks, the patient is considered for long term monitoring and the number of viral copies is measured periodically for a year, and
wherein if a patient does not have greater than 50 copies/mL after one year, the patient is considered to be cured of HIV-1.

2. The method of claim 1, wherein the HDAC inhibitor is selected from the group consisting of SAHA, valproic acid, butyric acid, and pharmaceutically acceptable salts thereof.

3. The method of claim 1, wherein the HAART comprises the administration of an integrase inhibitor, at least two reverse transcriptase inhibitors, and at least one entry inhibitor.

4. The method of claim 3, wherein the integrase inhibitor is raltegravir.

5. The method of claim 1, wherein the entry inhibitor is a CCR5 inhibitor.

6. The method of claim 1, wherein the HAART comprises the administration of a protease inhibitor.

7. The method of claim 1, the HDAC is romidepsin or vorinostat.

8. The method of claim 1, wherein the HAART comprises maraviroc, abacavir, zidovudine (AZT), and an integrase inhibitor selected from the group consisting of raltegravir, elvitegravir and dolutegravir.

9. The method of claim 1, wherein the HDAC inhibitor is butyric acid or a pharmaceutically acceptable salt thereof.

10. A method for treating an HIV-1-positive patient, comprising:
a) treating the patient with HAART until viral loads drop below 50 copies/mL;
b) while maintaining HAART, administering a combination of prostratin or a prostratin analog and an HDAC inhibitor,
c) while maintaining treatment with HAART and a combination of prostratin or a prostratin analog and an HDAC inhibitor, further administering a second HDAC inhibitor for a time between one week and ten weeks,
d) repeating steps b) and c) for a minimum of seven cycles,
e) stopping all therapy, and
f) periodically measuring the number of viral copies in the patient's serum,
wherein if a patient does not have greater than 50 copies/mL after a minimum of two weeks, the patient is considered for long term monitoring and the number of viral copies is measured periodically for a year, and
wherein if a patient does not have greater than 50 copies/mL after one year, the patient is considered to be cured of HIV-1.

11. The method of claim 10, wherein the HDAC inhibitor is selected from the group consisting of SAHA, valproic acid, butyric acid, and pharmaceutically acceptable salts thereof.

12. The method of claim 10, wherein the HAART comprises the administration of an integrase inhibitor, at least two reverse transcriptase inhibitors, and at least one entry inhibitor.

13. The method of claim 12, wherein the integrase inhibitor is raltegravir.

14. The method of claim 10, wherein the entry inhibitor is a CCR5 inhibitor.

15. The method of claim 10, wherein the HAART comprises the administration of a protease inhibitor.

16. The method of claim 10, the HDAC is romidepsin or vorinostat.

17. The method of claim 10, wherein the HAART comprises maraviroc, abacavir, zidovudine (AZT), and an integrase inhibitor selected from the group consisting of raltegravir, elvitegravir and dolutegravir.

18. The method of claim 10, wherein the HDAC inhibitor is butyric acid or a pharmaceutically acceptable salt thereof.

19. The method of claim 1, wherein a cytotoxic agent that targets HIV-producing cells is administered to the patient concurrently with, or after the patient has been administered the prostratin, bryostatin-1, or a prostratin or bryostatin analog and, optionally, an HDAC inhibitor.

20. The method of claim 19, wherein the cytotoxic agent that targets HIV-producing cells is a radiolabeled antibody.

21. The method of claim 20, wherein the radiolabeled antibody is administered after the patient has been administered the prostratin, bryostatin-1, or a prostratin or bryostatin.

22. The method of claim 21, wherein the radio-immunotherapy comprises the administration of radiolabeled antibodies targeting one or more viral antigen selected from the group consisting of HIV's gp120 and gp41 envelope proteins.

23. The method of claim 21, wherein the radio-immunotherapy comprises the administration of radiolabeled antibodies, wherein the radiolabels are selected from the group consisting of bismuth 213 and rhenium 188.

24. The method of claim 21, wherein the radio-immunotherapy comprises the administration of a monoclonal antibody to HIV's gp120 or gp41 envelope proteins, which antibody is tagged with bismuth 213 or rhenium 188.

25. The method of claim 21, wherein the antibody is an IgG antibody, an IgM antibody, or an an IgA antibody, or a fragment thereof, or a domain-deleted antibody.

26. The method of claim 21, wherein the dose of the radioisotope is between 1-500 mCi.

27. The method of claim 21, wherein the radio-immunotherapy that targets HIV producing cells is a radiolabeled peptide.

28. The method of claim 21, wherein the radio-immunotherapy that targets HIV producing cells is a radiolabeled aptamer.

29. A composition comprising an integrase inhibitor, an entry inhibitor, prostratin or a prostratin analog, an HDAC inhibitor, and two or more compounds selected from the group consisting of NRTIs and NNRTIs.

30. The composition of claim 29, wherein the composition comprises:
a) prostratin or a prostratin analog,
b) sodium butyrate,
c) raltegravir,
d) two NRTIs or a combination of an NRTI and an NNRTI, and
e) romidepsin or vorinostat.
